(19) United States
(12) Patent Application Publication
(10) Pub. No.: US 2018/0021259 A1
(43) Pub. Date: Jan. 25, 2018

(54) DYE-STABILIZED NANOPARTICLES AND METHODS OF THEIR MANUFACTURE AND THERAPEUTIC USE

(71) Applicant: Memorial Sloan Kettering Cancer Center, New York, NY (US)

(72) Inventors: Daniel A. HELLER, New York, NY (US); Yosef SHAMAY, New York, NY (US)

(73) Assignee: Memorial Sloan Kettering Cancer Center, New York, NY (US)

(21) Appl. No.: 15/549,985

(22) PCT Filed: Feb. 9, 2016
(86) PCT No.: PCT/US16/17153

§ 371 Date: Aug. 9, 2017

Related U.S. Application Data
(60) Provisional application No. 62/114,507, filed on Feb. 10, 2015.

(51) Int. Cl. A61K 9/16; A61K 31/44; A61K 31/337; A61K 31/565; A61K 31/519; A61K 31/553; A61K 31/635; A61K 31/42; A61K 31/4166; A61K 31/436; A61K 31/4184; A61K 31/4155; A61K 31/343; A61K 31/404; A61K 31/4745; A61K 31/517; A61K 47/22

(52) U.S. Cl. CPC A61K 9/1617; A61K 9/1694; A61K 47/22; A61K 31/44; A61K 31/337; A61K 31/565; A61K 31/519; A61K 31/553; A61K 31/635; A61K 31/42; A61K 31/4166; A61K 31/436; A61K 31/4184; A61K 31/4155; A61K 31/343; A61K 31/404; A61K 31/4745; A61K 31/517; G06F 19/706

(57) ABSTRACT

Described herein are nanoparticles which are largely made of (e.g., 90 wt. %) hydrophobic drugs and are stabilized by water soluble dyes. The nanoparticles can range in size from 30 nm to 150 nm and have highly negative surface charge (e.g., −55 mV). These nanoparticles are highly soluble in water, stable for days in PBS buffer and can be easily lyophilzed and reconstituted in water. Using quantitative self-assembly prediction calculations, topochemical molecular descriptors were identified and validated as highly predictive indicators of nano-assembly, nanoparticle size, and drug loading. The resulting nanoparticles selectively targeted kinase inhibitors to caveolin-1-expressing human colon cancer and autochthonous liver cancer models to yield striking therapeutic effects while avoiding pERK inhibition in healthy skin. The nanoparticles exhibited remarkable anti-tumor efficacy in vitro and in vivo in models of hepatocellular carcinoma.

### CROSS REFERENCE TO RELATED APPLICATION

This application claims the benefit of U.S. Application Ser. No. 62/114,507 filed on Feb. 10, 2015, the disclosure of which is hereby incorporated by reference in its entirety.

### GOVERNMENT SUPPORT

This invention was made with government support under grant number DP2-HD075698 awarded by the National Institutes of Health (NIH), grant number P30 CA008748 awarded by the National Cancer Institute (NCI), and grant number TG-MCB-130013 awarded by the National Science Foundation (NSF). The government has certain rights in this invention.

### FIELD OF THE INVENTION

This invention relates generally to nanoparticles and methods of their manufacture and therapeutic use. In particular embodiments, the invention relates to dye-stabilized nanoparticles for the treatment of cancer and other diseases.

### BACKGROUND

Many FDA approved and non-approved small molecule drugs suffer from poor water solubility, rapid clearance and relatively low concentration at site of disease. In cancer patients, systemically-delivered chemotherapy is often highly toxic, limiting the dose. In addition, potentially therapeutic new molecules are often too toxic to deliver using conventional routes, preventing their further development. Even as new molecularly targeted therapies are increasingly reaching the clinic, it is apparent that even such drugs often exhibit serious side-effects due to off-target responses. The use of nanotechnology to treat advanced cancers promises the reduction of toxic side-effects and improved efficacy (Lammers et al., Journal of controlled release, 161.2 (2012): 175-187). Nanoparticle therapeutics currently in the clinic attenuate some of the side-effects of chemotherapies. For instance, the liposomal drug doxorubicin reduces the cardiotoxicity of the encapsulated doxorubicin (Tacar et al., Journal of pharmacy and pharmacology, 65.2 (2013): 157-170). Paclitaxel reduces the incidence of neutropenia (Gradishar, Expert opinion on pharmacotherapy, (2006): 1041-1053). Most nanoparticulate formulations use macromolecule scaffolds or lipid bilayers (Lammers et al., British journal of cancer, (2008): 392-397).

Cyanine dyes are well known in the art to track therapeutic delivery. However, cyanine dyes at concentrations above 0.5% in water are known to self-assemble into aggregates and form chromatic liquid crystals, thereby limiting the efficacy of the therapeutic (Harrison et al., Journal of physical chemistry, 100.6 (1996): 2310-2321; Würthner et al., Angewandte Chemie International Edition, 50.15 (2011): 3376-3410). Hydrophobic interactions, along with weak attractions between aromatic rings of molecules (π-π interactions) cause molecular stacking. Because this stacking can occur with any number of molecules, aggregation can begin at any concentration, and many chromatic liquid crystals do not appear to exhibit the equivalent of a critical micelle concentration. Such aggregation is called isodesmic because it occurs at all concentrations. However, the aggregates formed at low concentrations are not large enough to align, and, at larger concentrations, aggregate size increases into supra-molecular assemblies.

A limitation of targeted nanoparticle drug carrier design is that complex synthetic schemes are often required, resulting in low loadings and higher barriers to clinical translation. The synthesis of nanoscale drug delivery vehicles is highly dependent on drug chemistry, and synthetic strategies seldom benefit from a priori information. This can also result in processes that are often unpredictable and based on trial-and-error methods. Moreover, low drug loadings and encapsulation efficiencies are common in most types of nanoparticle formulations, including liposomes, polymer micelles, and protein-based nanoparticles.

Crossing the vascular endothelial barriers remains a major challenge for developing efficient, targeted nanoparticle drug delivery systems for cancer therapy. Recently, caveolae-mediated targeting has been proposed as a strategy to facilitate endothelial penetration at tumor sites. Caveolae are specialized plasmalemmal vesicles which traffic material into and across the cell. Caveolin-mediated tumor targeting has been demonstrated using specific antibodies targeting caveolin-1. Interestingly, highly sulfated aromatic polymers can bind to caveolin through electrostatic and hydrophobic interactions.

Although caveolin-mediated tumor targeting might be advantageous because it does not require complex molecular recognition moieties such as antibodies, no methods exist that are able to develop a drug delivery strategy incorporating this chemistry as development of targeted nanoparticle drug carriers often requires complex synthetic schemes involving both supramolecular self-assembly and chemical modification. These processes are generally difficult to predict, execute, and control.

To improve materials properties in disparate fields, computational methods have been developed to guide synthetic strategies. For example, in drug carrier design, quantitative structure-property relationship (QSPR) calculations have been used to find molecular descriptors which correlate with in vivo performance, and molecular dynamics simulations have been used to understand nanoparticle supramolecular chemistry. However, these quantitative approaches have not yet enabled appreciable predictive power to facilitate the synthesis of drug carrier nanomaterials.

There exists a need for an easily tracked therapeutic platform that can encapsulate many classes of hydrophobic drugs at high concentrations, provide high anti-tumor efficacy, and provide predictability and control.

### SUMMARY OF INVENTION

Described herein is a targeted drug delivery system which is accurately and quantitatively predicted to self-assemble into nanoparticles based on the molecular structures of precursor molecules that are the drugs themselves. The drugs assemble with the aid of sulfated indocyanines into particles with ultra-high drug loadings of up to 90%. The nanoparticles can range in size from 30 nm to 150 nm and have highly negative surface charge (e.g., −55 mV). These nanoparticles are highly soluble in water, stable for days in PBS buffer and can be easily lyophilzed and reconstituted in water. The nanoparticles exhibited remarkable anti-tumor efficacy in vitro and in vivo in models of hepatocellular carcinoma. Using quantitative self-assembly prediction calculations, topochemical molecular descriptors were identified and validated as highly predictive indicators of nano-assembly, nanoparticle size, and drug loading. The resulting nanoparticles selectively targeted kinase inhibitors to caveolin-1-expressing human colon cancer and autochthonous liver cancer models to yield striking therapeutic effects while avoiding pERK inhibition in healthy skin.

In one aspect, the invention is directed to a dye-stabilized nanoparticle composition comprising: one or more hydrophobic drugs; and one or more sulfate-containing indocyanine dyes, wherein the composition is in the form of nanoparticles having diameter (e.g., average diameter) within a range from 30 nm to 150 nm.

In certain embodiments, the one or more hydrophobic drugs makes up at least 80 wt. % of the composition, e.g., at least 85 wt. %, e.g., at least 90 wt. %, e.g., at least 95 wt. %. In certain embodiments, the one or more sulfate-containing indocyanine dyes makes up no more than 20 wt. % of the composition, e.g., 15 wt. % or less, e.g., 10 wt. % or less, e.g., 5 wt. % or less; e.g., and wherein the total of the one or more dyes makes up at least about 5 wt. % of the composition, e.g., at least 10 wt. %.

In certain embodiments, the nanoparticles have a diameter within a range from 40 nm to 100 nm.

In certain embodiments, the one or more hydrophobic drugs are selected from Table 2. In certain embodiments, the one or more hydrophobic drugs is a kinase inhibitor. In certain embodiments, the one or more hydrophobic drugs comprises a fluorine (F). In certain embodiments, the one or more hydrophobic drugs comprises one or more members selected from the group consisting of sorafenib, paclitaxel, docetaxel, MEK162, etoposide, lapatinib, nilotinib, crizotinib, fulvestrant, vemurafenib, bexorotene, camptothecin, Mek Azd, talazoparib, GSK214, luminespib, forskolin, ABT737, tacrolimus, BMS-777607, tanespimycin, everolimus, trametinib, navitoclax, celecoxib, avagacestat, dutasteride, enzalutamide, regorafenib, RO4929097, valrubicin, and combinations thereof. In certain embodiments, the one or more hydrophobic drugs comprises one or more indole groups.

In certain embodiments, the one or more sulfate-containing indocyanine dyes comprises one or more members selected from Table 6. In certain embodiments, the one or more sulfate-containing indocyanine dyes comprises IR783. In certain embodiments, the one or more sulfate-containing dyes comprises a cyanine dye.

In certain embodiments, the nanoparticles are formed via nanoprecipitation.

In certain embodiments, the nanoparticles have a highly negative surface charge.

In certain embodiments, the highly negative surface charge is −20 mV or more negative, e.g., between −20 mV and −100 mV, e.g., −55 mV.

In certain embodiments, the one or more hydrophobic drugs remain associated with the one or more sulfate-containing indocyanine dyes without covalent bonding.

In certain embodiments, the dye is not covalently bonded to the drug, nor is it linked to the drug via a covalently-bonded linking moiety.

In certain embodiments, the dye-stabilized nanoparticle composition further comprises a carrier.

In another aspect, the invention is directed to a method of treating a disease or condition, the method comprising administering the dye-stabilized nanoparticle composition of any one of claims 1 to 18 to a subject suffering from or susceptible to the disease or condition.

In certain embodiments, the disease or condition is a member selected from the group consisting of cancer (e.g., sarcoma, carcinoma, etc.), inflammatory disease, rheumatoid arthritis, inflammatory bowel disease, lupus, age-related macular degeneration.

In certain embodiments, the administered dye-stabilized nanoparticle composition obviates skin rashes.

In certain embodiments, the method further comprises irradiating the dye-stabilized nanoparticle composition.

In another aspect, the invention is directed to a method of making the dye-stabilized nanoparticle composition, the method comprising: introducing a first solution into a second solution in a drop-wise manner while stirring (or otherwise mixing or agitating) the second solution, wherein the first solution comprises the one or more hydrophobic drugs in a solvent, and wherein the first solution is a buffered aqueous solution (e.g., 0.02 M to 0.05 M sodium bicarbonate, e.g., PBS) comprising the one or more sulfate-containing indocyanine dyes.

In certain embodiments, the solvent is DMSO or ethanol (e.g., at a concentration from about 1 mg/ml to about 100 mg/ml, e.g., about 5 mg/ml to about 25 mg/ml, e.g., about 10 mg/ml).

In certain embodiments, the one or more sulfate-containing indocyanine dyes has a total dye concentration from about 1 mg/ml to about 3 mg/ml.

In certain embodiments, the method further comprises performing centrifugation and/or sonication to collect the formed nanoparticles.

In another aspect, the invention is directed to a method for predicting self-assembly of a dye-stabilized nanoparticle composition, the method comprising: providing a molecular structure of a drug; generating, by a processor a computing device (e.g., a computer programmed to generate, e.g., in silico), a set of one or more molecular descriptors for the drug, wherein the set of molecular descriptors comprises one or more of (i), (ii), (iii), and (iv) as follows: (i) a first molecular descriptor identifying a likelihood the drug will self-assemble with a dye to generate a dye-stabilized nanoparticle composition comprising the drug and the dye; (ii) a second molecular descriptor identifying a maximal quantity of drug that can be loaded into a/the dye-stabilized nanoparticle composition comprising the drug and the dye; (iii) a third molecular descriptor identifying (e.g., quantifying) hydrophobicity of the drug; and (iv) a fourth molecular descriptor identifying a diameter of a/the dye-stabilized nanoparticle composition comprising the drug and the dye.

In certain embodiments, the set of molecular descriptors comprises one, two, three, or all four of (i), (ii), (iii), and (iv).

In certain embodiments, the first molecular descriptor is a leading eigenvalue of a Burden matrix, e.g., weighted by intrinsic state(s), e.g., wherein the eigenvalue is greater than 6.99, e.g., wherein the Burden matrix is a topochemical index to score the molecular structure of the drug based on at least a geometrical complexity, a bond order, and heteroatoms of the molecular structure, e.g., wherein the generating of the set of one or more molecular descriptors comprises computing one or more eigenvalues of the Burden matrix.

In certain embodiments, a logarithmic value of the third molecular descriptor is at least 4.5.

In certain embodiments, the method further comprises manufacturing the dye-stabilized nanoparticle composition comprising the drug and the dye.

In certain embodiments, the dye-stabilized nanoparticle composition has a drug loading no greater than the maximal quantity identified by the second molecular descriptor.

In certain embodiments, the dye-stabilized nanoparticle composition is determined to self-assemble based on at least one or more members of the set of molecular descriptors.

In certain embodiments, the drug is a hydrophobic drug. In certain embodiments, he drug comprises F. In certain embodiments, the drug comprises one or more indole groups.

In certain embodiments, the dye is a sulfate-containing indocyanine dye.

### Definitions

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration”: The term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In certain embodiments, administration is oral. Additionally or alternatively, in certain embodiments, administration is parenteral. In certain embodiments, administration is intravenous.

“Biocompatible”: The term “biocompatible”, as used herein is intended to describe materials that do not elicit a substantial detrimental response in vivo. In certain embodiments, the materials are “biocompatible” if they are not toxic to cells. In certain embodiments, materials are “biocompatible” if their addition to cells in vitro results in less than or equal to 20% cell death, and/or their administration in vivo does not induce inflammation or other such adverse effects. In certain embodiments, materials are biodegradable.

“Biodegradable”: As used herein, “biodegradable” materials are those that, when introduced into cells, are broken down by cellular machinery (e.g., enzymatic degradation) or by hydrolysis into components that cells can either reuse or dispose of without significant toxic effects on the cells. In certain embodiments, components generated by breakdown of a biodegradable material do not induce inflammation and/or other adverse effects in vivo. In certain embodiments, biodegradable materials are enzymatically broken down. Alternatively or additionally, in certain embodiments, biodegradable materials are broken down by hydrolysis. In certain embodiments, biodegradable polymeric materials break down into their component polymers. In certain embodiments, breakdown of biodegradable materials (including, for example, biodegradable polymeric materials) includes hydrolysis of ester bonds. In certain embodiments, breakdown of materials (including, for example, biodegradable polymeric materials) includes cleavage of urethane linkages.

“Carrier”: As used herein, “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

“Dye-stabilized nanoparticle”: As used herein, the term “dye-stabilized nanoparticle” or indocyanine nanoparticles includes nanoparticles formed from sulfated indocyanine precursors which are non-covalently bound to one or more hydrophobic drugs. Note that “nanoparticles” is not necessarily a solid or particulate, and does not necessarily have a uniform diameter or shape. Nanoparticles are understood to have an average diameter from about 1 nm to about 1000 nm.

“Subject”: As used herein, the term “subject” includes humans and mammals (e.g., mice, rats, pigs, cats, dogs, and horses). In many embodiments, subjects are be mammals, particularly primates, especially humans. In certain embodiments, subjects are livestock such as cattle, sheep, goats, cows, swine, and the like; poultry such as chickens, ducks, geese, turkeys, and the like; and domesticated animals particularly pets such as dogs and cats. In certain embodiments (e.g., particularly in research contexts) subject mammals will be, for example, rodents (e.g., mice, rats, hamsters), rabbits, primates, or swine such as inbred pigs and the like.

“Therapeutic agent”: As used herein, the phrase “therapeutic agent” refers to any agent that has a therapeutic effect and/or elicits a desired biological and/or pharmacological effect, when administered to a subject.

“Treatment”: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In certain embodiments, treatment may be of a subject who has been diagnosed as suffering from the relevant disease, disorder, and/or condition. In certain embodiments, treatment may be of a subject known to have one or more susceptibility factors that are statistically correlated with increased risk of development of the relevant disease, disorder, and/or condition.

Drawings are presented herein for illustration purposes, not for limitation.

### BRIEF DESCRIPTION OF DRAWINGS

The foregoing and other objects, aspects, features, and advantages of the present disclosure will become more apparent and better understood by referring to the following description taken in conduction with the accompanying drawings, in which:

FIGS. 1A-1F shows the chemical structure and name of dyes that can be used for nanoparticle preparation.

FIG. 2 depicts the preparation scheme for dye encapsulated nanoparticles composed of sorafenib (SFB) and the cyanine dye IR783.

FIGS. 3A-E depict methods for characterization of the nanoparticles.

FIGS. 4A-C show differential update of IR783-SFB evaluated by fluorescence microscopy.

FIGS. 5A-C depict increased accumulation of dye-encapsulated nanoparticles in liver tumors.

FIG. 6A depicts experimental design of a hepatocellular carcinoma (HCC) anti-tumor assessment.

FIG. 6B shows improved response of the livers to the IR783-SFB over orally administered (PO) SFB.

FIG. 7A shows images of livers from the three groups at the day of termination, or 49 days after inoculation.

FIG. 7B depicts fluorescence images of GFP expressing in the treatment groups.

FIG. 7C shows liver weight and tumor volume as a measurement for tumor burden of all the liver in the groups.

FIG. 7D shows liver weight and tumor volume as a measurement for tumor burden of all the liver in the groups.

FIG. 7E depicts representative H&E staining of liver tissue samples from the 3 groups.

FIGS. 8A-B show a photodynamic therapy application of IP783-SFB nanoparticles.

FIGS. 9A-9D show indocyanine-drug self-assembly.

FIG. 9A shows images of precipitating (left) and suspending (right) indocyanine-drug mixtures.

FIG. 9B shows absorption spectra of indocyanine (top left) upon serial dilution with buffer, (bottom left) upon serial dilution with DMSO, (top right) upon introduction of drugs which resulted in precipitate formation, and (bottom right) upon introduction of drugs which resulted in suspensions.

FIG. 9C shows scanning electron microscopy (SEM) images of indocyanine-drug nanoparticles. Scale bar=100 nm.

FIG. 9D shows nanoparticle size as a function of drug:indocyanine ratio.

FIGS. 10A-10H shows prediction and analysis of indocyanine nanoparticle formation by QSAP and MD.

FIG. 10A shows a training set of 16 drugs experimentally determined to precipitate or form nanoparticles with indocyanine and plotted according to the molecule's leading eigenvalue of the Burden matrix descriptor SpMAX4_Bh(s).

FIG. 10B shows the maximum stable drug:indocyanine ratios of drugs which formed nanoparticles, plotted against the molecule's SpMAX5_Bh(s) eigenvalue. Data is presented as mean±s.d.

FIG. 10C shows SpMAX4_Bh(s) eigenvalues of 280 drugs. Arrows indicate experimental validation of stability. Solid arrows=drugs that precipitated with indocyanine, Dashed arrows=drugs that formed stable nanoparticles with indocyanine.

FIG. 10D shows a training set of 8 drug molecules plotted by experimentally-determined nanoparticle size formed with indocyanine, and correlated to the molecular descriptor Getaway R4e. R²=0.84.

FIG. 10E shows prediction of nanoparticle size of a validation set of drug molecules based on the Getaway-R4e descriptor.

FIG. 10F shows snapshots from the top clusters acquired from all-atom molecular dynamics simulations of drug-indocyanine systems. Indocyanine, sorafenib, and taselisib molecules are shaded respectively.

FIG. 10G shows reduction in solvent accessible surface area of drug due to the presence of indocyanine.

FIG. 10H shows number of intra-nanoparticle hydrogen bonds.

FIGS. 11A-11E show internalization of indocyanine nanoparticles in 2D and 3D cell culture.

FIG. 11A shows fluorescence micrographs of INP internalization in different cell lines.

FIG. 11B shows inhibition of internalization mechanisms with chemical inhibitors, including cyclodextrin (CD) and filipin III inhibitors of caveolae, chloropromazine (CBZ) inhibitor of clathrin-mediated endocytosis, and bromo-sulfophthalein (SBM) inhibitor of OAT1-3.

FIG. 11C shows indocyanine nanoparticle uptake in cell lines, quantified by fluorescence intensity correlated with CAV1 expression (R²=0.86).

FIG. 11D shows nanoparticle uptake in a co-culture of two cell lines.

FIG. 11E shows CAV1 staining in tumor spheroids composed of two different cell lines (left) and nanoparticle fluorescence in tumor spheroids (right). Scale bar=20 μm.

FIGS. 12A-12F show indocyanine nanoparticle targeting and efficacy in MYC-driven autochthonous murine hepatic tumor model.

FIG. 12 A shows CAV1 and CD31 staining in liver sections 3 weeks (top) and 6 weeks (bottom) after hydrodynamic injection. Arrow indicates tumor nodule. Scale bar=50 μm.

FIG. 12B shows fluorescence images of livers with multiple GFP-positive tumor nodules 24 h after administration of nanoparticles. NIR=INP indocyanine emission, GFP=cancer fluorescence.

FIG. 12C shows macroscopic images of livers extracted 60 days after tumor initiation (top), GFP fluorescence in livers (middle), and representative H&E stained liver sections (bottom). Scale bar=50 μm. Bottom: Fluorescence of GFP in livers.

FIG. 12D shows liver weights.

FIG. 12E shows tumor volume as measured in the livers if detectable.

FIG. 12G shows quantification of GFP fluorescence.

FIG. 13A-13D shows anti-tumor efficacy in HCT116 colon cancer model.

FIG. 13A shows immunohistochemical staining of tumor section for CAV1 and CD31 expression in HCT116 xenografts 2 weeks after inoculation.

FIG. 13B shows tumor growth inhibition in response to nanoparticles or controls (n=6).

FIG. 13C shows IHC staining of proliferation marker Ki67 on HCT116 tumor slices at day 35.

FIG. 13D shows IHC staining of pERK as a biomarker for drug activity in skin and tumor tissue.

FIG. 14 shows indocyanine-drug suspensions. Photographs of drug/indocyanine mixtures at increasing drug:indocyanine ratios. The drug concentration was fixed at 2 mg/ml and the indocyanine IR783 concentrations were 0, 0.005, 0.01, 0.03 and 0.1 mg/ml (from left to right). In the case of fulvestrant, indocyanine concentrations were 0.0001, 0.005 and 0.001 mg/ml. All tubes were centrifuged for 1 min at 3000 g before imaging.

FIGS. 15A-15F show indocyanine nanoparticle characterization.

FIG. 15A shows nanoparticle diameters measured with dynamic light scattering (DLS).

FIG. 15B shows nanoparticle zeta potential measured with electrophoretic light scattering.

FIG. 15C shows transmission electron microscopy (TEM) images of indocyanine nanoparticles. Scale Bar=100 nm.

FIG. 15D shows atomic force microscopy (AFM) images of indocyanine nanoparticles. Scale Bar=100 nm.

FIG. 15E shows nanoparticle stability in growth medium containing serum evaluated by DLS.

FIG. 15F shows DLS data from nanoparticles synthesized using other sulfated indocyanine compounds, IR806 and IR820.

FIGS. 16A and 16B show QSAP training sets. Molecular structures of drugs used in the training set for QSAP determination of indocyanine nanoparticle formation.

FIGS. 17A-17D show molecular descriptors of nanoparticle formation. Four molecular descriptors exhibiting highly significant correlations (>0.85) with experimental data of nanoparticle formation.

FIG. 18 shows contribution of fluorine to the formation of indocyanine nanoparticles. Structures of celecoxib and valdexoxib with their SpMAX4 eigenvalues (top). Photographs of drug-indocyanine mixtures (bottom).

FIGS. 19A-19F show molecular dynamics simulations of indocyanine nanoparticles.

FIGS. 19A-19B show initial configurations for (FIG. 19A) sorafenib-indocyanine and (FIG. 19B) taselisib-indocyanine simulations containing 12 drug molecules, 4 dye molecules, ions, and water each.

FIG. 19C show normalized radial particle density histograms for the most probable configurations of sorafenib-indocyanine and taselisib-indocyanine, plotted as a function of distance from the particle center.

FIG. 19D shows change in solvent accessible drug surface area plotted as a function of REMD time.

FIG. 19E shows total number of intra-nanoparticle (non-water) hydrogen bonds plotted as a function of REMD time.

FIG. 19F shows number of drug-indocyanine hydrogen bonds plotted as a function of REMD time. In (FIG. 19C) through (FIG. 19F), sorafenib-indocyanine data is denoted by grey curves and taselisib-indocyanine data is black.

FIGS. 20A and 20B show differential expression of CAV1 in cell lines.

FIG. 20A shows CAV1 gene expression profile in human cell lines, obtained from CCLE database (Entrez ID: 857).

FIG. 20B shows immunohistochemical staining for CAV1 in cell lines. Scale bar=50 μM. Images of HL60, U138, and TIME cell lines were obtained from the Human Protein Atlas database.

FIGS. 21A and 21B show indocyanine nanoparticle targeting of tumor spheroids.

FIG. 21A shows characterization of tumor spheres with: (left) SEM, (center) H&E histolochemical stain, and (right) immunohistochemical stain for CAV1.

FIG. 21B shows fluorescence microscopy of near-infrared dye emission of tumor spheroids after 20 min and 120 min of incubation. Scale bar=20 μm.

FIGS. 22A-22C show biodistribution of indocyanine nanoparticles.

FIG. 22A shows fluorescence images of whole body in vivo and organs ex vivo 20 min after i.v. administration of sorafenib INPs, measured by IVIS.

FIG. 22B shows fluorescence images of organs ex vivo 24 h after i.v. administration of 3 different indocyanine nanoparticles.

FIG. 22C shows biodistribution of indocyanine nanoparticles quantified from ex vivo fluorescence images as total fluorescence efficiency normalized by organ weight.

FIGS. 23A-23F show sorafenib INP targeting in autochthonous liver cancer model. Images of resected livers 21 days after hydrodynamic injection of Sleeping Beauty transposon vectors encoding c-Myc and mutant β-catenin coupled to GFP.

FIG. 23A shows color photograph of the resected liver.

FIG. 23B shows fluorescence image of GFP channel emission.

FIG. 23C shows hematoxylin and eosin stain.

FIG. 23D shows immunohistochemical stain for CD31.

FIG. 23E shows immunohistochemical stain for CAV1. All scale bars=200 μM.

FIG. 23F shows accumulation of indocyanine (IR783) and indocyanine nanoparticles in livers of the genetically modified mouse model (top three livers) vs. normal livers (bottom two livers) (left) and GFP fluorescence images (right).

FIGS. 24A-24E show sorafenib INP imaging in uveal melanoma liver metastasis model.

FIG. 24A shows photograph taken at 4 weeks after inoculation.

FIG. 24B shows hematoxylin and eosin stain of tumor tissue.

FIG. 24C shows immunohistochemical stain for CD31 at 3 weeks after inoculation.

FIG. 24D shows an immunohistochemical stain for CAV1 3 weeks after inoculation. Arrow indicates the tumor margin. All scale bars=150 μM.

FIG. 24E shows images of livers from the uveal melanoma model 24 h after administration of nanoparticles to tumors (Top) 2 weeks after inoculation and (Bottom) 4 weeks after inoculation. (Left) Near-infrared channel. (Center) GFP fluorescence channel. (Right) Brightfield image.

FIGS. 25A and 25B show trametinib INP targeting in HCT116 colon cancer model.

FIG. 25A shows near-infrared fluorescence of mice imaged in vivo 24 h after i.v. administration of trametinib INPs.

FIG. 25B shows biodistribution of trametinib INPs 24 h after i.v. administration, calculated from ex vivo fluorescence images as total fluorescence efficiency divided by organ weight.

FIGS. 26A-26C and 27A-27C show apoptosis assessment in Cal33 cells 24 hours after irradiation.

FIGS. 28A-28D show photodynamic therapy (PDT) in 3 dimensional (3D) tumor spheroids and mice.

FIG. 28A shows characterization of SK-136 tumor spheroids.

FIG. 28B shows penetration of particles into the sphere within 2 h.

FIGS. 28C and 28D show that radiation of a single sphere transforms the dye into radicals.

FIG. 29 shows that the irradiated sphere appears dead and does not grow on plastic as the non-irradiated sphere does.

FIGS. 30A-30C show instrumentation setup for In Vivo Near Infrared Fluorescence (NIRF) imaging and irradiation.

FIGS. 31A and 31B show an in vivo PDT experiment. A mouse bearing 3LL s.c tumor were i.v injected with IR783-Sorafenib nanoparticles (3 mg/kg) and the tumor was irradiated with a 808 nm laser (25 J/cm²) for 2 min. The mouse was imaged with IVIS before and after irradiation and the disappearance of the dye was evidence for radical formation.

FIGS. 32A and 32B show an in vivo PDT experiment. Similar to FIGS. 31A and 31B, mice bearing 3LL s.c tumor were i.v injected with IR783-Sorafenib nanoparticles (3 mg/kg) and the tumor was irradiated with a 808 nm laser (25 J/cm²) for 2 min. The mice were imaged with IVIS before and after irradiation and the disappearance of the dye was evidence for radical formation.

FIGS. 33A and 33B show the mice imaged in FIGS. 32A and 34B at 5 hours after irradiation.

FIG. 34 is a block diagram of an example network environment for use in the methods and systems for analysis of spectrometry data, according to an illustrative embodiment.

FIG. 35 is a block diagram of an example computing device and an example mobile computing device, for use in illustrative embodiments of the invention.

### DETAILED DESCRIPTION

It is contemplated that methods of the claimed invention encompass variations and adaptations developed using information from the embodiments described herein.

Throughout the description, where compositions are described as having, including, or comprising specific components, or where methods are described as having, including, or comprising specific steps, it is contemplated that, additionally, there are compositions of the present invention that consist essentially of, or consist of, the recited components, and that there are methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

The present disclosure provides a nanoparticle platform which can encapsulate many classes of hydrophobic drugs via self-assembly with sulfated water soluble cyanine fluorescent dyes. The dye nanoparticles (DNPs) can be highly-stabilized drug colloids synthesized by non-covalent self-assembly which form stable homogenous nanoparticles. In general, nanoparticles require careful control of the intermolecular interactions via modulation of the dye and drug molecules themselves, stoichiometry, solvents, salinity, pH, and physical conditions including shear forces, temperature, and mixing conditions.

Described herein is a caveolin-targeted nanoparticle drug delivery platform constructed via self-assembly of small molecule sulfated indocyanine precursors where nanoparticle formation can be accurately predicted using quantitative information from the structure of the encapsulated drug. Via quantitative self-assembly prediction (QSAP) calculations, three molecular descriptors were identified to predict (1) which drugs would assemble with indocyanine into nanoparticles, (2) maximal drug loadings, and (3) nanoparticle size with an accuracy of 15 nm. Moreover, this approach also revealed molecular structural features that enable self-assembly and nanoparticle formation. Notably, the resulting indocyanine nanoparticles (INPs) were found to encapsulate drugs with ultra-high loadings of up to 90%. Via all-atom replica exchange molecular dynamics simulations, large differences were found in intra-particle densities which correlated with hydrogen bonding between drug molecules within the particles, giving an initial picture of the nanoparticle interiors.

The targeted drug delivery properties of two such nanoparticles were assessed: nanoparticles encapsulating tyrosine kinase inhibitors sorafenib and trametinib. Notably, selective tumor uptake and exceptional net anti-tumor efficacies were found in a genetically modified mouse model for hepatocellular carcinoma and a xenograft model for human colorectal cancer. The nanoparticles prevented the inhibition of ERK phosphorylation in the skin, demonstrating that this targeting strategy exhibits strong therapeutic benefits and may obviate skin rashes—a major side-effect of kinase inhibitors.

The disclosed technology, in certain embodiments, provides for manufacturing dye-encapsulated nanoparticles using a nano-precipitation method. In certain embodiments, the method comprises preparing an aqueous buffer solution containing about 0.02-0.05 M sodium bicarbonate in water with 1-3 mg/ml dye. In certain embodiments, the hydrophobic drugs are dissolved in either DMSO or ethanol at a concentration of about 10 mg/ml and introduced into the aqueous buffer solution by slow drop-wise addition while stirring and mixing. The nanoparticles can be collected by several centrifugations followed by short ultra-sonication using a small diameter probe. It is hypothesized that the sulfate groups in the water soluble dyes yield a highly negative surface charge and are responsible for the stability and water solubility of the nanoparticles. Moreover, without wishing to be limited, it is thought that the combination of hydrophobic aromatic indole groups with the negatively charged sulfate groups may be responsible for the facile internalization into endothelial and cancer cells through caveolar endocytosis while evading macrophage uptake.

In certain embodiments, the presently disclosed nanoparticle platform results in remarkably high drug content/loading (e.g., greater than 85%) and vastly improves the targeting and potential activity of the drug at the disease site due to the highly negative charge of the nanoparticle.

In certain embodiments, the drug used in the dye-stabilized nanoparticles includes one or more of the following: sorafenib, paclitaxel, docetaxel, MEK162, etoposide, lapatinib, nilotinib, crizotinib, fulvestrant, vemurafenib, bexarotene and camptothecin.

In certain embodiments, the DNPs can be administered intravenously in a saline solution, for example, a PBS buffer. In certain embodiments, the formulation may also contain 5% sucrose for stability under lyophilization. In the methods of the disclosed technology, any route of administration may be utilized including, for example, parenteral (e.g., intravenous), oral, topical, subcutaneous, peritoneal, intra-arterial, inhalation, vaginal, rectal, nasal, introduction into the cerebrospinal fluid, or instillation into body compartments. In certain embodiments, administration is oral. Additionally or alternatively, in certain embodiments, administration is parenteral. In certain embodiments, administration is intravenous.

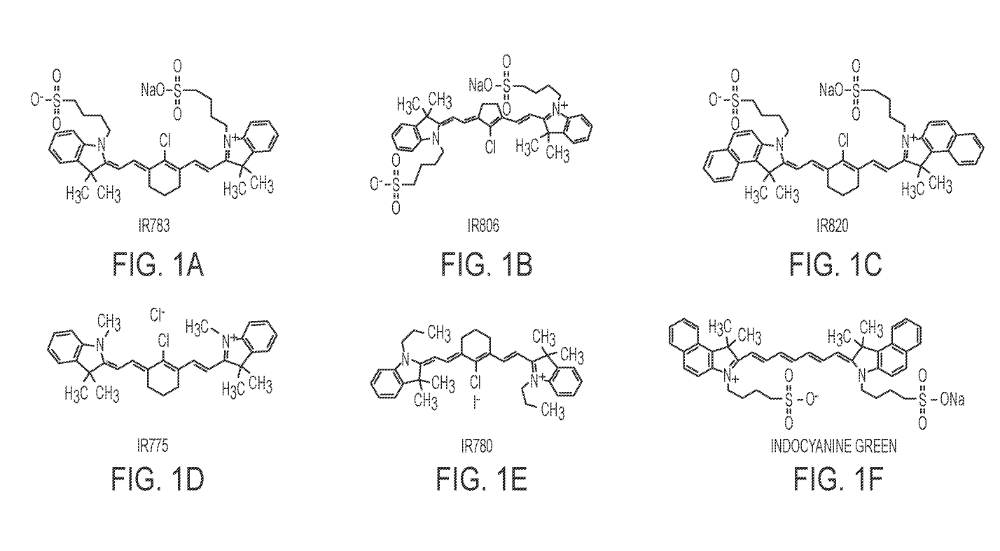

In certain embodiments, fluorophores, or “dyes”, that can be used for nanoparticle preparation include, for example, the following: IR783 (2-[2-[2-Chloro-3-[2-[1,3-dihydro-3,3-dimethyl-1-(4-sulfobutyl)-2H-indol-2-ylidene]-ethylidene]-1-cyclohexen-1-yl]-ethenyl]-3,3-dimethyl-1-(4-sulfobutyl)-3H-indolium) as depicted in FIG. 1A, IR806 (2-[2-[2-chloro-3-[2-[1,3-dihydro-3,3-dimethyl-1-(4-sulfobutyl)-2H-indol-2-ylidene]-ethylidene]-1-cyclopenten-1-yl]-ethenyl]-3,3-dimethyl-1-(4-sulfobutyl)-3H-indolium) as depicted in FIG. 1B, IR820 (2-[2-[2-Chloro-3-[[1,3-dihydro-1,1-dimethyl-3-(4-sulfobutyl)-2H-benzo[e]indol-2-ylidene]-ethylidene]-1-cyclohexen-1-yl]-ethenyl]-1,1-dimethyl-3-(4-sulfobutyl)-1H-benzo[e]indolium) as depicted in FIG. 1C, IR775 (2-[2-[2-Chloro-3-[2-(1,3-dihydro-1,3,3-trimethyl-2H-indol-2-ylidene)-ethylidene]-1-cyclohexen-1-yl]-ethenyl]-1,3,3-trimethyl-3H-indolium) as depicted in FIG. 1D, IR780 (2-[2-[2-Chloro-3-[(1,3-dihydro-3,3-dimethyl-1-propyl-2H-indol-2-ylidene)ethylidene]-1-cyclohexen-1-yl]ethenyl]-3,3-dimethyl-1-propylindolium) as depicted in FIG. 1E, Indocyanine Green (IR125) (Sodium 4-[(2E)-2-{(2E,4E,6E)-7-[1,1-dimethyl-3-(4-sulfonatobutyl)-1H-benzo[e]indolium-2-yl]-2,4,6-heptatrien-1-ylidene}-1,1-dimethyl-1,2-dihydro-3H-benzo[e]indol-3-yl]-1-butanesulfonate) as depicted in FIG. 1F. In certain embodiments, other molecules that form aggregate structures, for example, J- and H-aggregates, can be used as dyes. In certain embodiments, a stabilizing molecule (i.e., a dye) can include aromatic moieties. In certain embodiments, a stabilizing molecule (i.e., a dye) is highly charged. In certain embodiments, the stabilizing molecule (i.e. a dye) is a sulfate containing conjugated polymethine dye. In certain embodiments, a stabilizing molecule (i.e., a dye) is water soluble.

In certain embodiments, the DNPs can be applied for the treatment of multiple disease types including but not limited to many cancers such as sarcomas and carcinomas, inflammatory diseases such as rheumatoid arthritis, inflammatory bowel disease, lupus, age-related macular degeneration, etc.

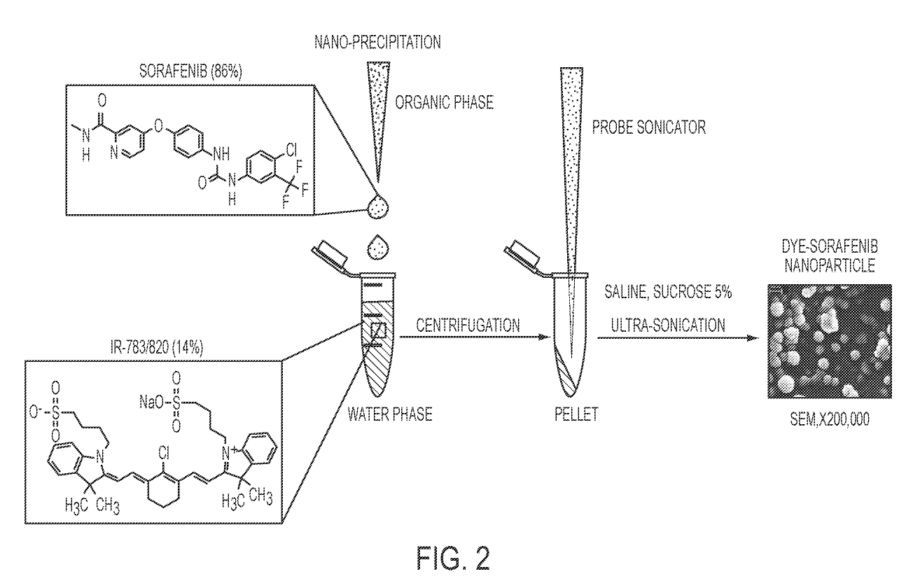

In certain embodiments, the DNPs comprise sorafenib (SFB) and a cyanine dye, such as IP783 as shown in FIG. 1A, and prepared as depicted in FIG. 2. FIG. 2 shows the preparation scheme of IR783-SFB nanoparticles composed of sorafenib and the cyanine dye IR783. The drug in DMSO is added drop wise into the dye solution in water at basic pH. In certain embodiments, the nanoparticles exhibit excellent aqueous stability including after reconstituting lyophilized material.

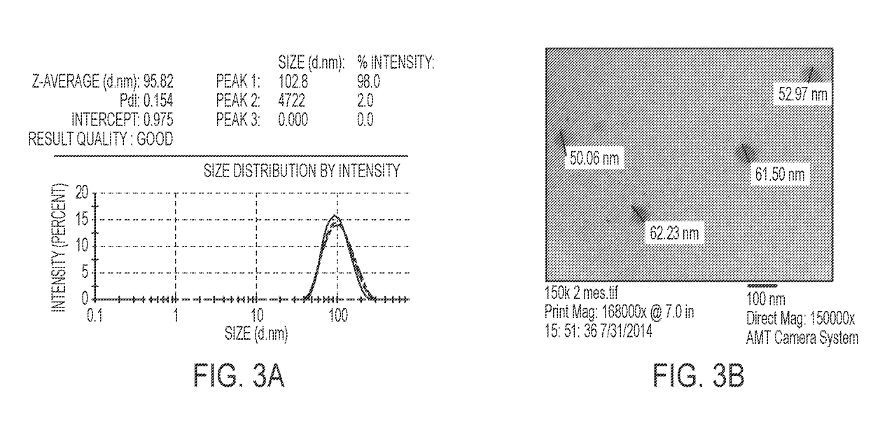

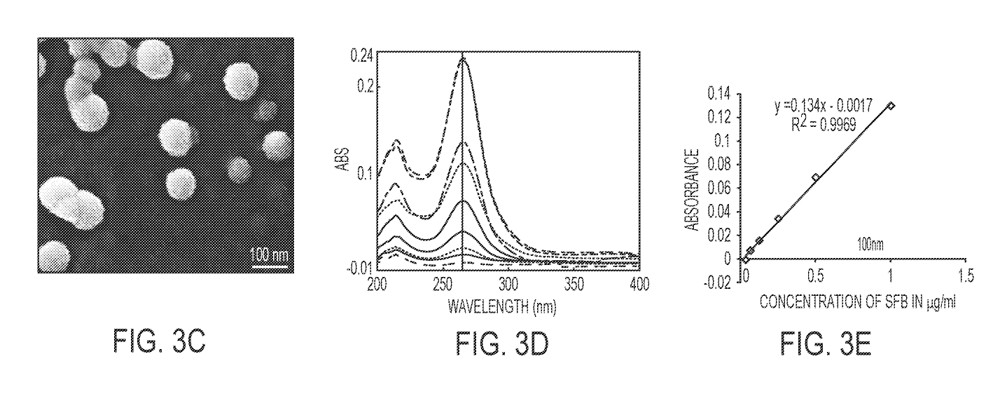

The DNPs can be characterized by dynamic light scatter (DLS) as shown in FIG. 3A, by transmitting electron microscope as shown in FIG. 3B, and/or by scanning electron microscope as depicted in FIG. 3C. In certain embodiments, the drug content of the DNPs can be characterized by absorbance spectroscopy with a UV-VIS spectrometer as shown in FIG. 3D. In certain embodiments, the calculation of the drug content can be calculated using the Beer-Lambert equation as depicted in FIG. 3E.

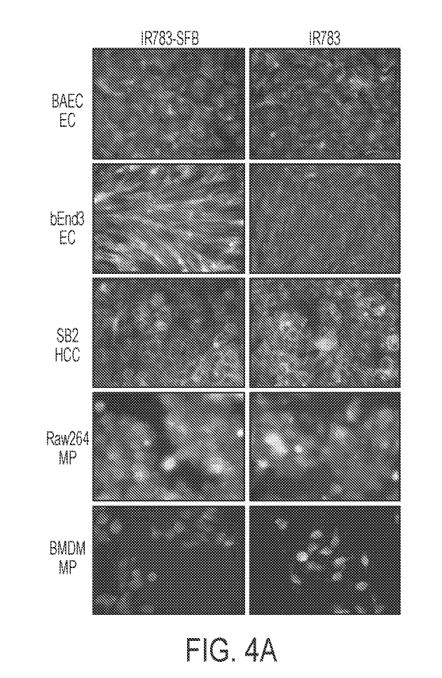

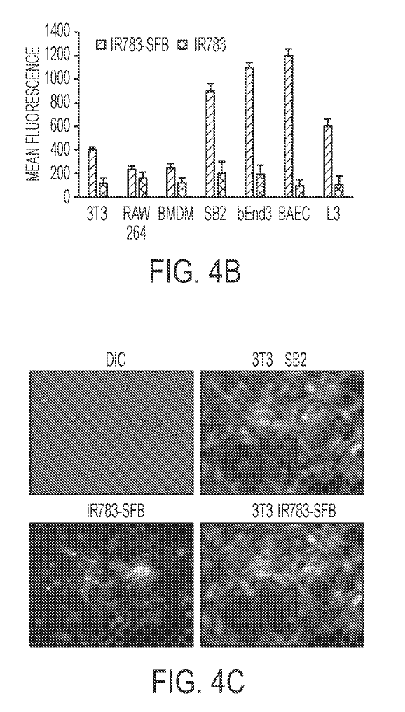

Differential uptake of IR783-SFB nanoparticles can be evaluated by fluorescence microscopy, as shown in FIGS. 4A-C. FIG. 4A shows 5 different cell lines that were incubated for 2 h with 30 μg/ml IR783-SFB particles (left panels) or equivalent amount of free IR783 dye (right panels). The cells that internalized the most nanoparticles were BAEC and bEnd3 endothelial cells and SB2 liver cancer cells. Bone marrow derived macrophages (BMDM), RAW264 macrophages, and 3T3 fibroblasts did take up the nanoparticles efficiently. The dye alone does not internalize in the cells. FIG. 4B depicts quantitative analysis of fluorescence intensity in the cell lines was conducted using ImageJ v1.40 (NIH, USA). The mean fluorescence intensities are expressed in arbitrary units per square micron. The increased uptake by endothelial cells and cancer cells as opposed to macrophages and fibroblasts is about 4 fold. FIG. 4C depicts confirmation of the differential uptake between cell lines, 3T3 fibroblasts (GFP) were co-cultured with SB2 liver cancer cells (RFP) to confluence and incubated with nanoparticles for 30 min. The cell morphology of the co-culture can be seen in bright light (upper left, DIC) and fluorescence channel (upper right). The nanoparticles fluorescence (cyan) is found specifically in the liver cancer cells (lower left and lower right).

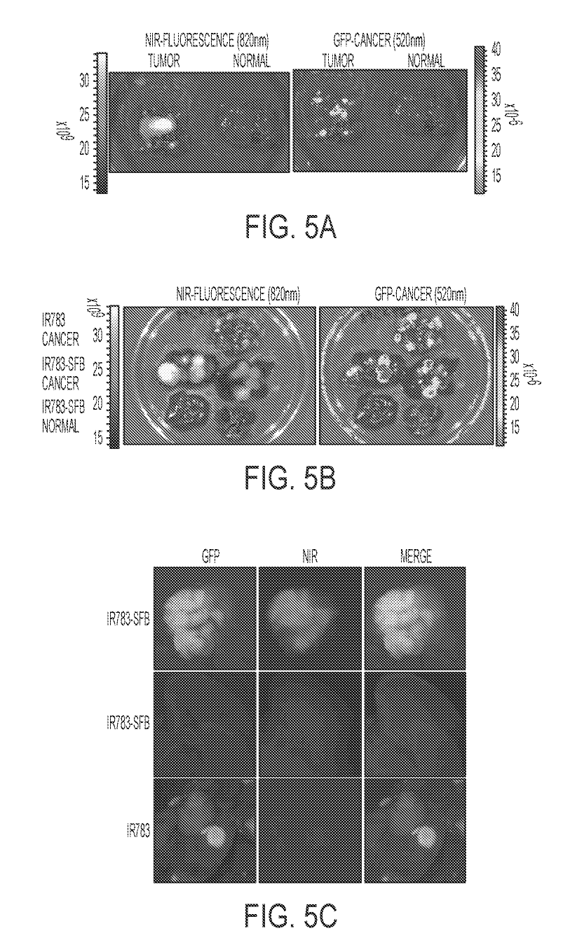

Fluorescent image-guided surgery is an emerging modality to visualize tumor margins by near IR-fluorescence probes with cancer specificity. In certain embodiments, the near-infrared (IR) dyes facilitate the tracking and imaging of the particle/drug distribution in the whole body and within the tumor. In certain embodiments, the DNPs accumulate in tumor tissue and can be used to assist IR fluorescent image guided surgery. In certain embodiments, the nanoparticles can exhibit increased accumulation in tumors, such as in liver tumors as shown in FIGS. 5A-C. In certain embodiments, the nanoparticles may exhibit increased accumulation endothelial cells. In certain embodiments, the nanoparticles can exhibit increased accumulation kidney and thyroid cancer cells. In certain embodiments, the nanoparticles offer a partial and/or complete removal of tumor lesion which can result in increased survival.

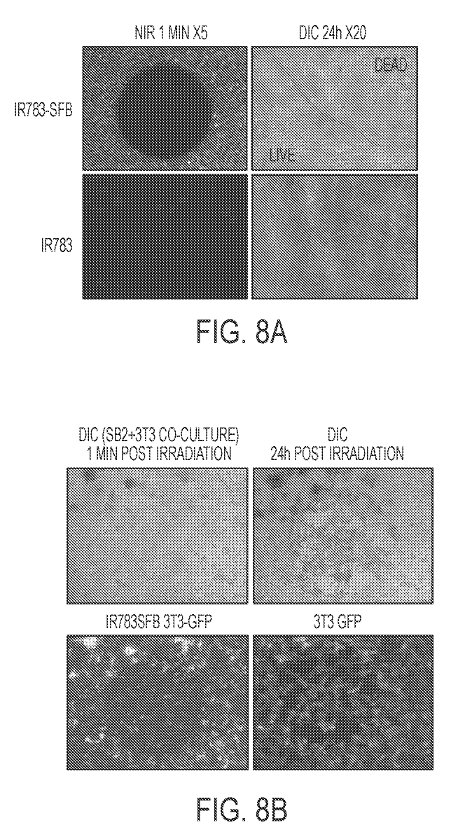

In certain embodiments, tissues (i.e., liver tissue) show a significant response to the IR783-SFB over orally administered (PO) SFB as shown in FIGS. 5A-B and 6A-E. In some embodiment, the DNPs can be used in photodynamic therapy applications as shown, for example, in FIGS. 8A-B. In certain embodiments, only the cancer cells are affected by the exposure to light irradiation during photodynamic therapy of DNPs.

### Experimental Examples

### Synthesis Methods of Selected Nanoparticles

In this example, the nanoparticles were synthesized by nano-precipitation. In this example concentrated hydrophobic drug solution in organic solvent was slowly introduced dropwise to a water phase which contains a water soluble sulfated organic dye. This method is often used to produce nanoparticles composed polymers or lipids, but in this case we used small molecule cyanine dyes. The size range of the resulting particles was between 20 and 300 nm with a polydispersity index of about 0.05-0.3 and a monodispersity of about 0.05-0.15. The particles were administered intravenously in a saline solution or PBS buffer, but many routes should be possible, including interperitoneally, subcutaneously, or intramuscularly. The injection media may also contain 5% sucrose for stability under lyophilization.

### Preparation of Indocyanine Nanoparticles

0.1 ml of each drug, dissolved in DMSO (10 mg/ml), was added drop-wise (20 per 15 sec) to a 0.6 ml aqueous solution containing IR783 (1 mg/ml) and 0.05 mM sodium bicarbonate under slight vortexing. The solution was centrifuged twice (20,000 G, 30 min) and re-suspended in 1 ml of sterile PBS. The suspension was ultrasonicated for 10 sec with a ⅛″ probe tip (Sonics & Materials) at 40% intensity. The nanoparticles were lyophilized in a 5% saline/sucrose solution. Absorbance spectra were acquired using a TECAN M1000 plate reader.

### Sorafenib-IR783 Nanoparticles:

Dye-encapsulated sorafenib nanoparticles (IR783-SFB) were synthesized using the nano-precipitation method as shown in FIG. 2. In this example, 0.1 ml of sorafenib dissolved in DMSO (10 mg/ml) was added drop-wise (20 μL per 15 sec) to a 0.4 aqueous IR783 solution (1 mg/ml) containing 0.05 mM sodium bicarbonate. The solution was centrifuged twice (20,000 G 30 min) and re-suspension in 1 ml of sterile PBS. The suspension of nanoparticles was sonicated for 10 sec with a probe sonicator at 40% intensity (Sonics). The resulted IR783-SFB nanoparticles had zeta potential of −52 mV and a size of 95 nm with a PDI of 0.1 as depicted in FIG. 3. By suspending the nanoparticles in lower volumes they were able to solubilize sorafenib up to 16 mg/ml in saline solution which is 2000 times better than free drug. The nanoparticles were easily lyophilized with a saline/sucrose 5% solution and reconstituted in water at this concentration.

### Paclitaxel IR820 Nanoparticles

Dye-encapsulated paclitaxel nanoparticles (IR820-PAX) were synthesized using a nano-precipitation method. Paclitaxel dissolved in ethanol (10 mg/ml) was added drop-wise (20 μL per 15 sec total of 0.15 ml) to 0.3 ml of IR 820 solution in water (2 mg/ml). The solution was centrifuged twice (20,000 G 20 min) and re-suspended in 1 ml of sterile PBS. The suspension of nanoparticles was sonicated for 10 sec with a probe sonicator at 40% intensity (Sonics). The paclitaxel-loaded nanoparticles had a zeta potential of −55 mV and an average diameter of 90 nm with PDI of 0.08. The nanoparticles were able to solubilize paclitaxel up to 12 mg/ml in saline solution which is a 1000 times better than free Paclitaxel and 2.4 fold more than FDA-approved albumin-stabilized paclitaxel. The nanoparticles were easily lyophilized and reconstituted in water at this concentration.

### Dye Combination Encapsulation of Etoposide and Captothecin

Dye encapsulated drug (etoposide or camptothecin) nanoparticles were prepared using a combination of heptamethine cyanine dyes, a water soluble dye (IR820), and a DMSO-soluble dye (IR775). The drugs (etoposide and camptothecin), dissolved in DMSO (10 mg/ml), was mixed with 2 mg/ml IR775 in DMSO to a total volume of 0.25 ml and then added dropwise to a concentrated solution of IR820 (3-4 mg/ml) in water. The solution was centrifuged twice (20,000 G 30 min) and re-suspension in 1 ml of sterile PBS. The suspension of nanoparticles was sonicated for 10 sec with a probe sonicator at 40% intensity (Sonics).

### Nanoparticle Characterization

Nanoparticles were characterized by Dynamic Light Scattering (DLS), Scanning Electron Microscopy (SEM) and Transmission electron microscopy (TEM) as shown in FIGS. 3A-3C. DLS and zeta potential measurements were conducted using a Zetasizer Nano ZS (Malvern). SEM was conducted using a Zeiss Supra 25 Field Emission scanning electron microscope. Samples were prepared by gold sputtering and critical point drying. For AFM measurements, nanoparticles were observed on a freshly cleaved mica surface using an Asylum NIFP 3D Bio with an Olympus AC240TS AFM probe. For TEM, a carbon-coated copper TEM grid (Ted Pella) was used with a JEOL 1200 EX transmission electron microscope operated at 80 kV.

The content of the drug was measured by UV-VIS absorbance at 260 nm or 280 nm (Sorafenib or Paclitaxel) as shown in FIGS. 3D-3E.

### In Vitro Uptake of Nanoparticles

The uptake of the sorafenib-IR783 nanoparticles (IR783-SFB) was tested in 6 cell types: endothelial cells (BAEC and bEnd3 cell line), liver HCC cells (SB2 cells, derived from the in vivo model), fibroblasts (3T3), and macrophages (BMDM and RAW264). The cells were incubated for 2 hours with 30 μg/ml and as a control with an equivalent amount of free IR783. The cells were washed and stained with Cell Mask (Life Sciences) for membrane staining and DAPI for nuclear staining. The cells were imaged with a florescence microscope equipped with an Indocyanine Green filter set, as depicted in FIGS. 4A-4B. Similar conditions were applied to image a co-culture experiment of fibroblasts and cancer cells as shown in FIG. 4C.

### Anti-Tumor Efficacy of Nanoparticles

Sorafenib-containing nanoparticles were tested in vivo for imaging and treatment of hepatocellular carcinoma in an orthotopic model. The nanoparticles were shown to accumulate specifically in liver tumors, as shown in FIG. 5. FVB mice were transfected by hydrodynamic injection on day 0 with plasmids to C-Myc and beta-catenin to inoculate liver tumors. FIG. 5A depicts mice bearing liver tumors and healthy mice were injected with IR783-SFB (for example, at 20 mg/kg) at day 21 after inoculation. After 24 hours the livers were extracted and imaged using an IVIS Preclinical In Vivo Imaging System (Perkin Elmer) in fluorescence imaging mode. A representative image of the near IR-fluorescence (left panel) shows IR783-SFB nanoparticle accumulation in small tumors while there is significantly lower accumulation in the normal liver. The tumors were also imaged by their GFP expression (right panel). FIG. 5B shows mice bearing liver tumors were injected with free dye (IR783) or dye-sorafenib nanoparticles (IR783-SFB) at day 32 after inoculation and imaged after 24 h. The near IR-fluorescence (left panel, middle row) shows IR783-SFB nanoparticle accumulation in tumors while the free dye (IR783) does not accumulate. The GFP expression in the tumors was also imaged (right panel). FIG. 5C shows images of the livers from the same experiment captured with a fluorescence stereoscope. Co-localization (right column) of the near IR channel of the nanoparticle (middle column) with the cancer GFP (left column) shows enhanced uptake of nanoparticles in tumors.

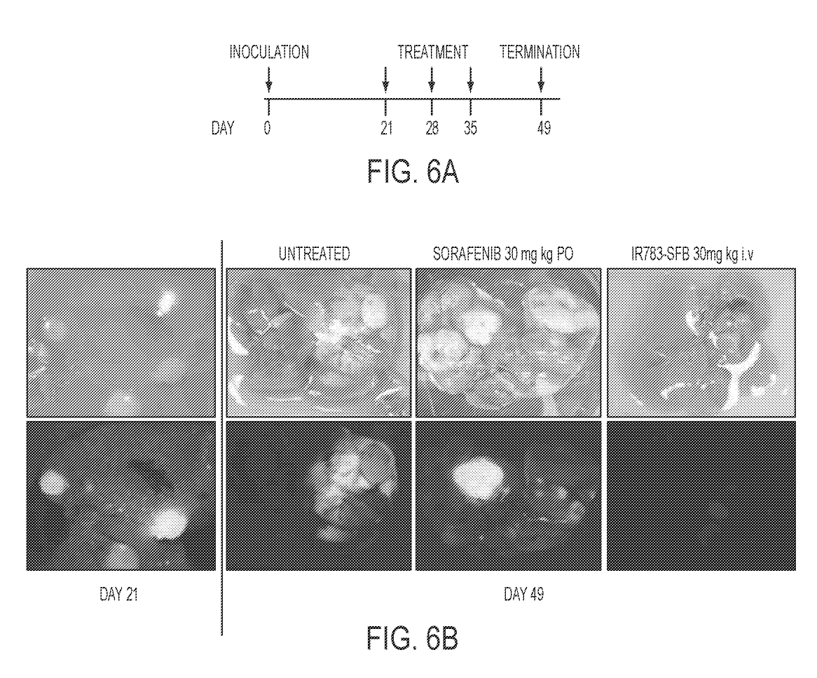

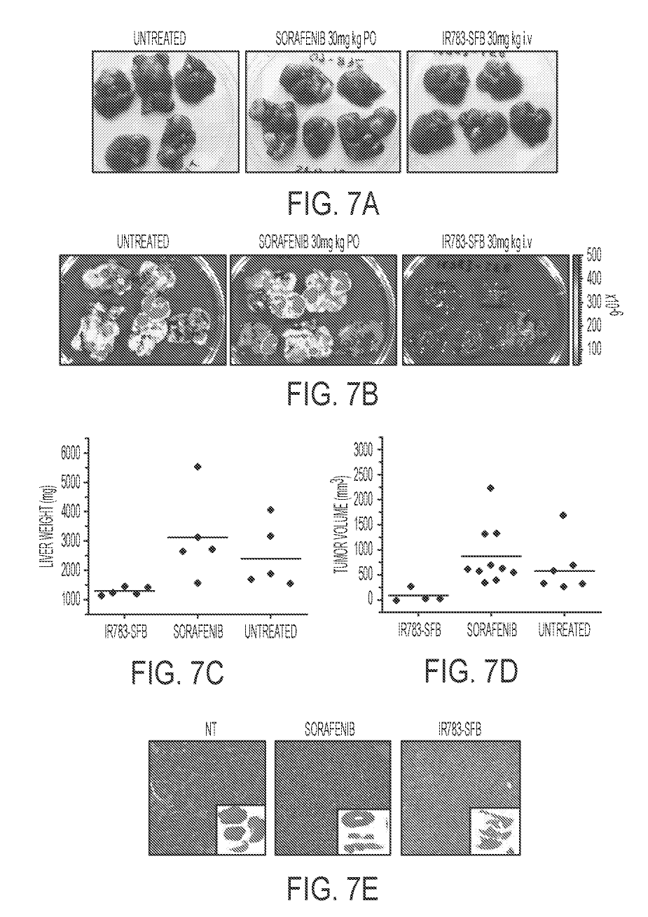

The nanoparticles shows remarkable efficacy as compared to sorafenib alone or albumin based nanoparticles, as depicted in FIGS. 6A-6B and FIGS. 7A-7E. FIG. 6A shows experimental design of a hepatocellular carcinoma (HCC) anti-tumor assessment. For example, here 20 FVB mice were administered a hydrodynamic injection on day 0 with plasmids to C-Myc and beta-catenin to initiate liver tumors. The efficacy of IR783-SFB was compared to oral SFB. Treatments started at day 21 after inoculation and were administrated once weekly for 3 weeks. FIG. 6B shows the experiment was terminated at day 59 and livers were extracted and imaged. Representative images of the livers show a significant response to the IR783-SFB over orally administered (PO) SFB.

FIGS. 7A-7E show an in vivo anti-tumor efficacy of the IR783-SFB nanoparticles on the HCC model. FIG. 7A depicts images of livers from the three groups at the day of termination (49 days after inoculation). FIG. 7B shows fluorescence images of GFP expressing in the treatment groups. FIGS. 7C and 7D depict liver weight and tumor volume as a measurement for tumor burden of all the liver in the groups. FIG. 7E shows representative H&E staining of liver tissue samples from the 3 groups.

### Photodynamic Therapy

The IR783-SFB nanoparticles were tested for the application of photodynamic therapy. The radiosensitizing properties of the dyes may provide an added therapeutic benefit of the targeted nanoparticles. Endothelial and cancer cells were incubated with the nanoparticles for 30 min and then washed and exposed to NIR light from a Xenon lamp for 1 min. After 24 h, the cells were imaged, showing dead and dying cells denoted by a clear change in cell morphology in the irradiated area as depicted in FIGS. 8A-8B. FIG. 8A depicts endothelial cells that were incubated for 30 min with NP and irradiated for 1 min with a xenon lamp (Xcite) using an ICG filter set (Chroma). The fluorescence image (upper left) shows a dark area where the irradiated area bleached the IR783 dye. After 24 h, the morphology of the irradiated area was altered and cells detached after washing (upper right). The free dye had no significant effect (lower panel). FIG. 8B shows co-cultures of fibroblasts (3T3-GFP) and SB2 cells (unlabeled) after 1 min irradiation. Bright light images (upper left) shows confluence of the co-culture.

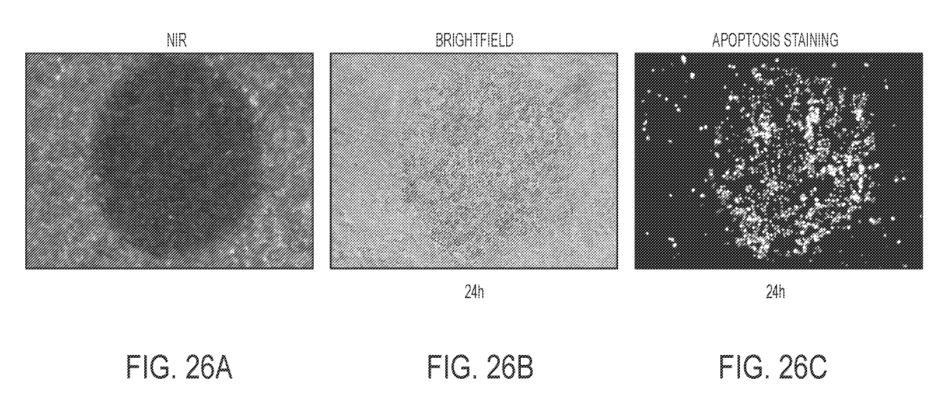

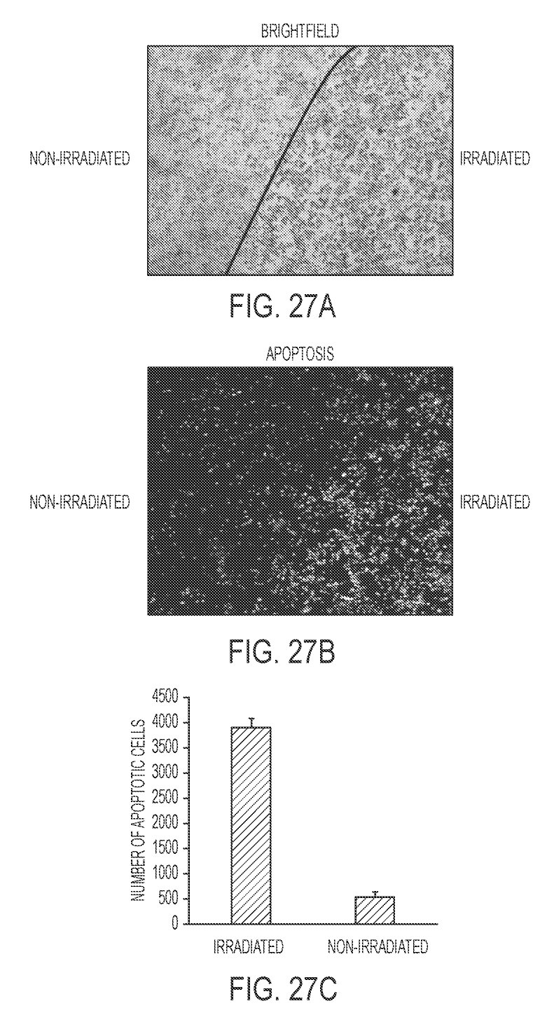

FIGS. 26A-26C and 27A-27C show apoptosis assessment in Cal33 cells 24 hours after irradiation.

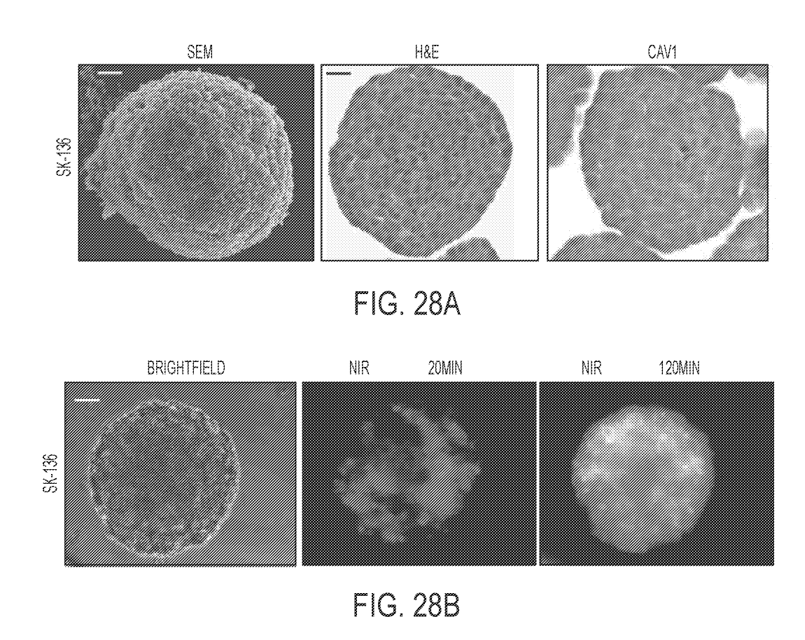

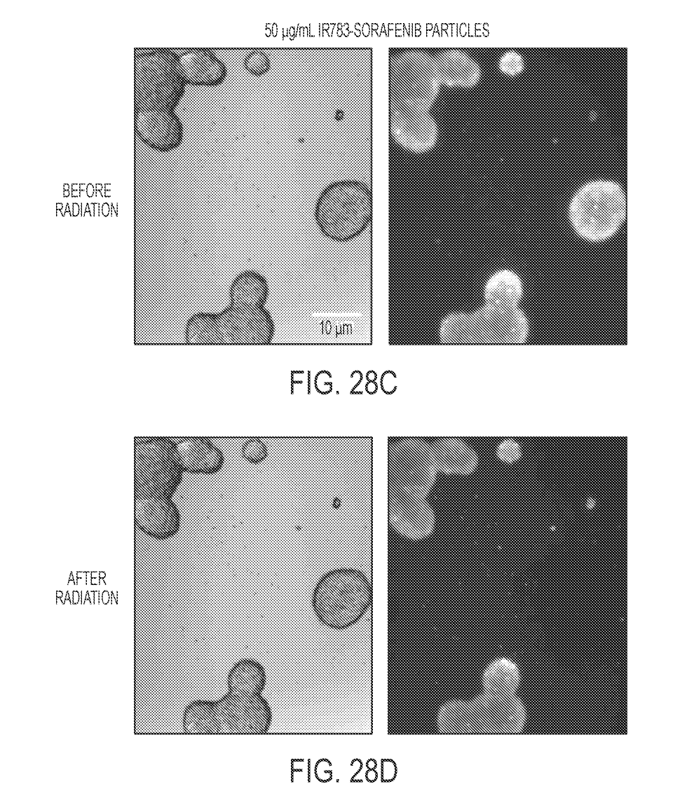

FIGS. 28A-28D show photodynamic therapy (PDT) in 3 dimensional (3D) tumor spheroids and mice.

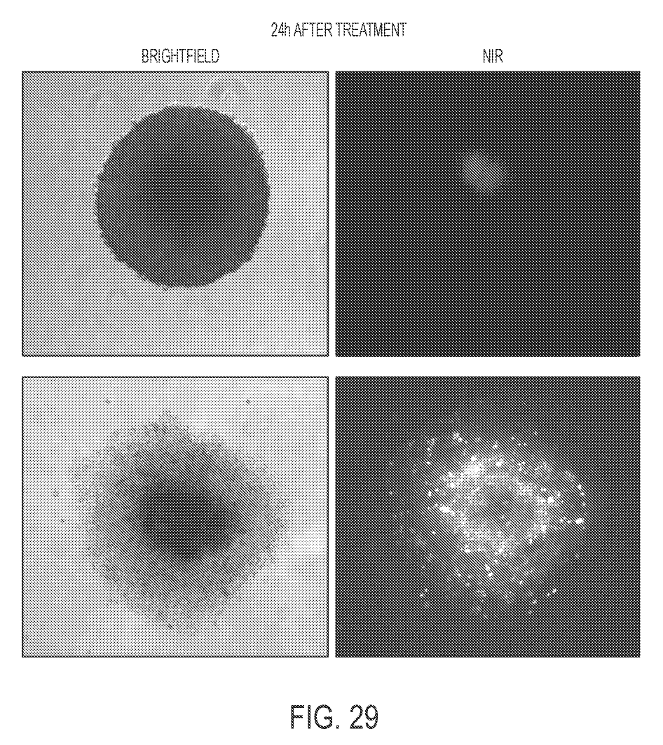

IR783-Sorafenib nanoparticles were tested for PDT experiments and showed that they only internalize in Caveolin-1 expressing cancer cells and induce cell death upon irradiation with an 808 nm laser (FIGS. 28A-28D and 29). FIG. 28A shows characterization of SK-136 tumor spheroids. FIG. 28B shows penetration of particles into the sphere within 2 h. FIGS. 28C and 28D show that radiation of a single sphere transforms the dye into radicals. FIG. 29 shows that the irradiated sphere appears dead and does not grow on plastic as the non-irradiated sphere does.

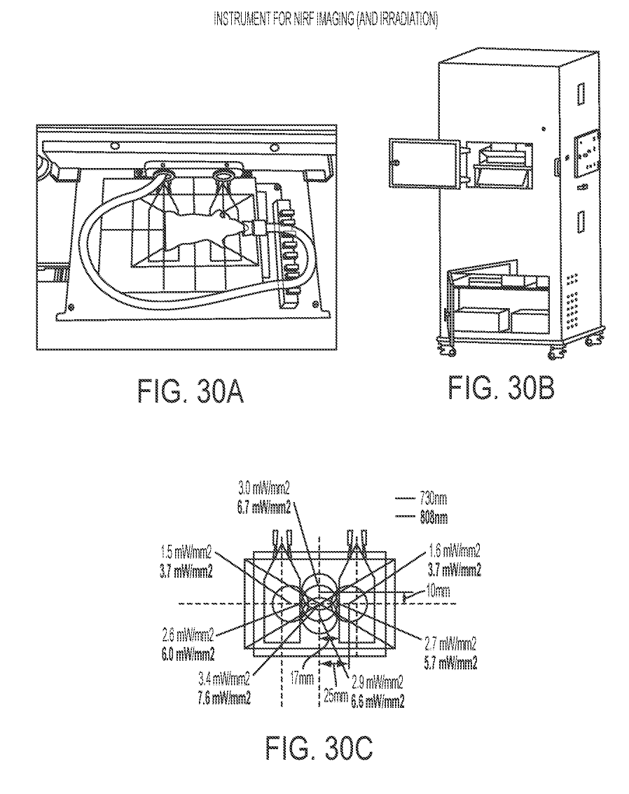

FIGS. 30A-30C show an exemplary instrumentation setup for In Vivo Near Infrared Fluorescence (NIRF) imaging and irradiation.

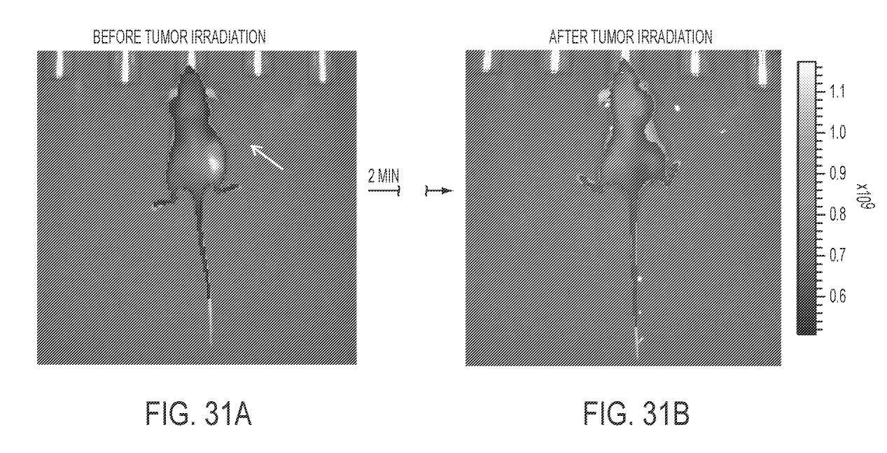

FIGS. 31A and 31B show an in vivo PDT experiment. A mouse bearing 3LL s.c tumor were i.v injected with IR783-Sorafenib nanoparticles (3 mg/kg) and the tumor was irradiated with a 808 nm laser (25 J/cm²) for 2 min. The mouse was imaged with IVIS before and after irradiation and the disappearance of the dye was evidence for radical formation.

Similar to FIGS. 31A and 31B, FIGS. 32A and 36B show mice bearing 3LL s.c tumor were i.v injected with IR783-Sorafenib nanoparticles (3 mg/kg) and the tumor was irradiated with a 808 nm laser (25 J/cm²) for 2 min. The mice were imaged with IVIS before and after irradiation and the disappearance of the dye was evidence for radical formation.

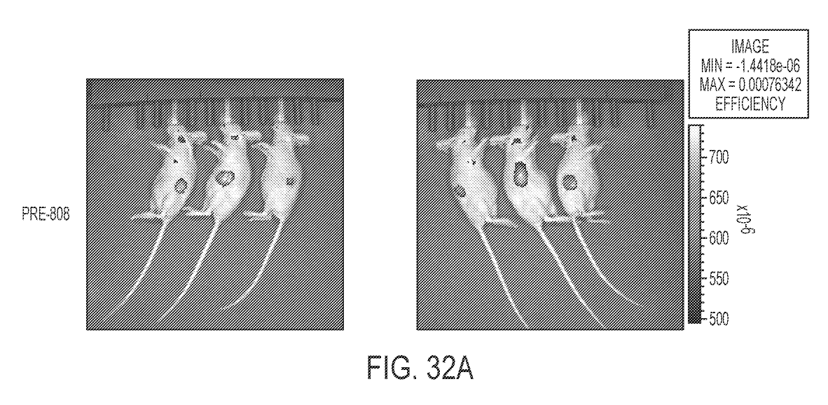

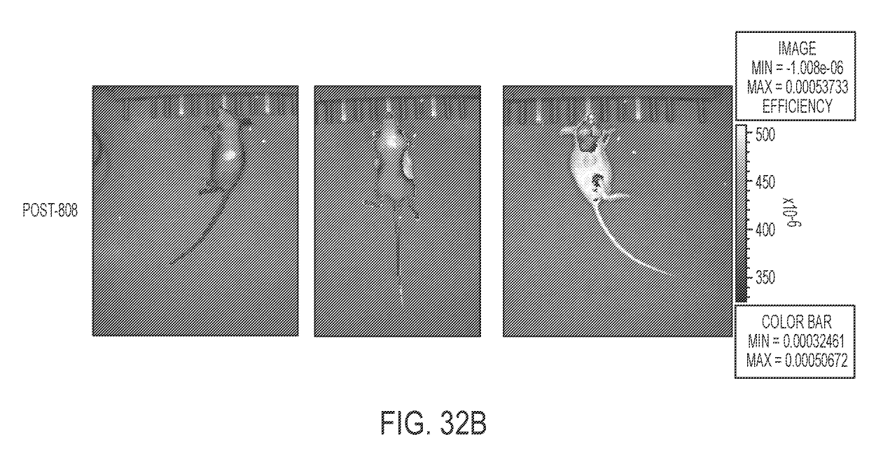

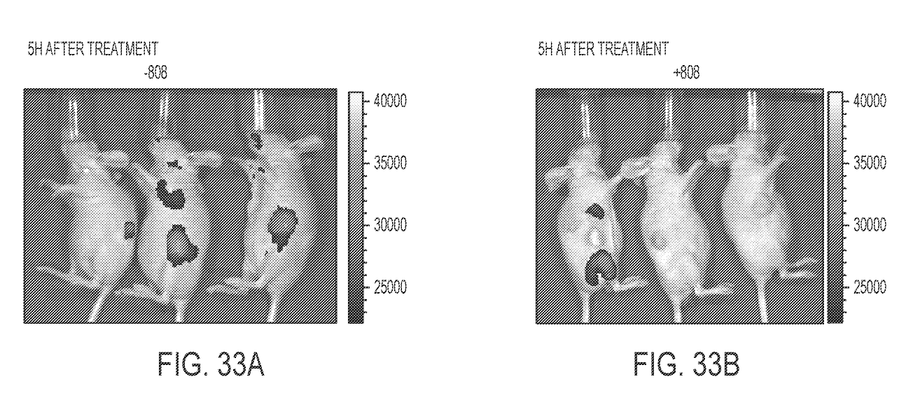

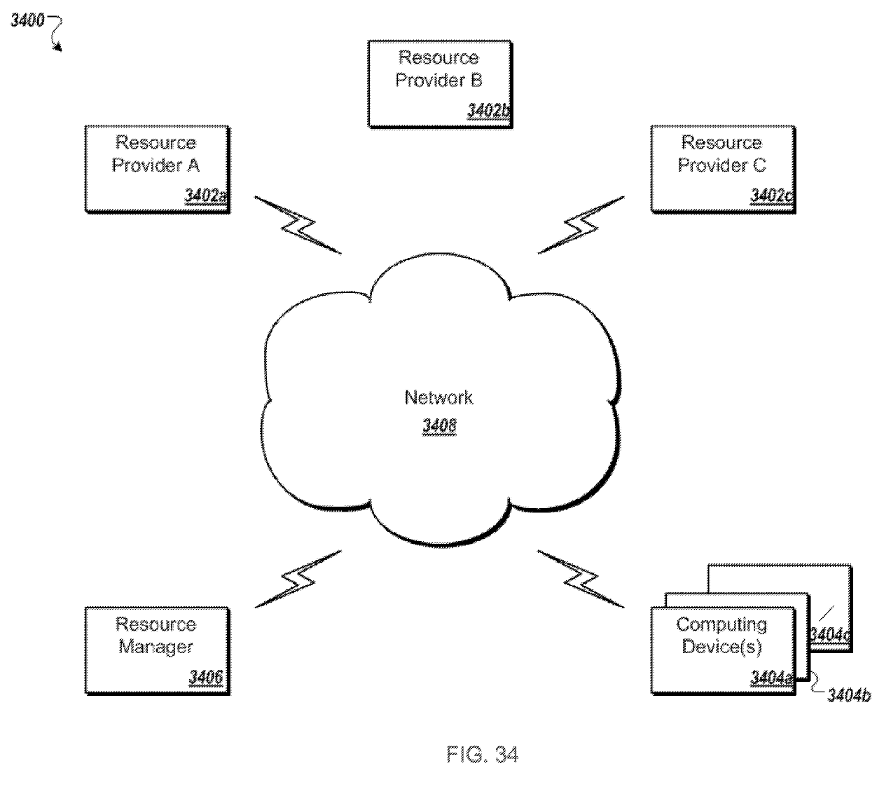

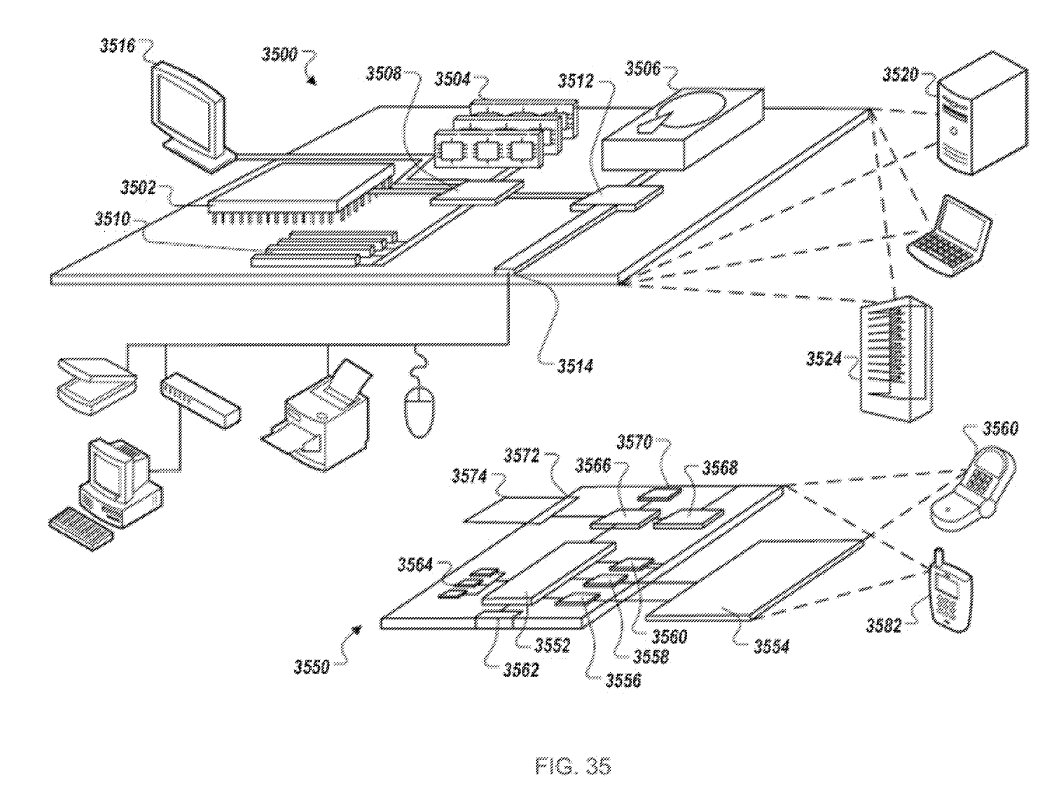

FIGS. 33A and 33B show the mice imaged in FIGS. 32A and 34B at 5 hours after irradiation.

Self-Assembly of Multiple Hydrophobic Drugs with Sulfated Indocyanine Compounds Using Nano-Precipitation Methods

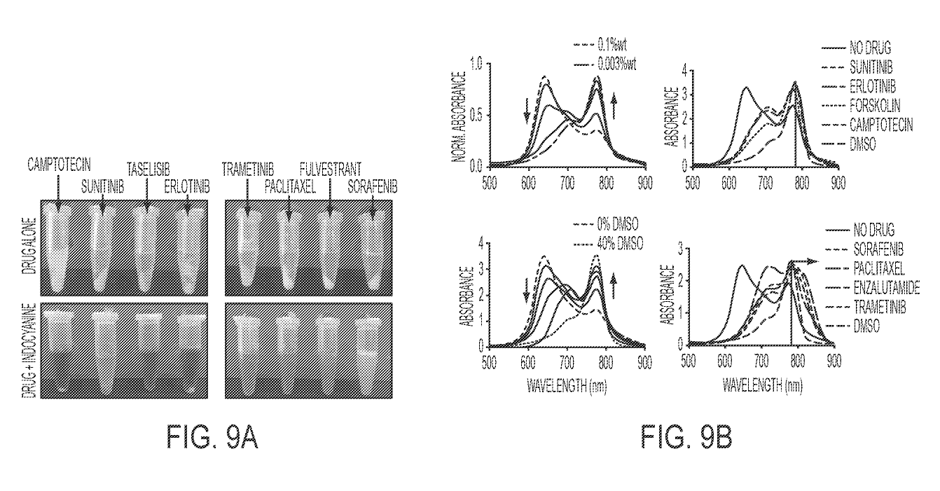

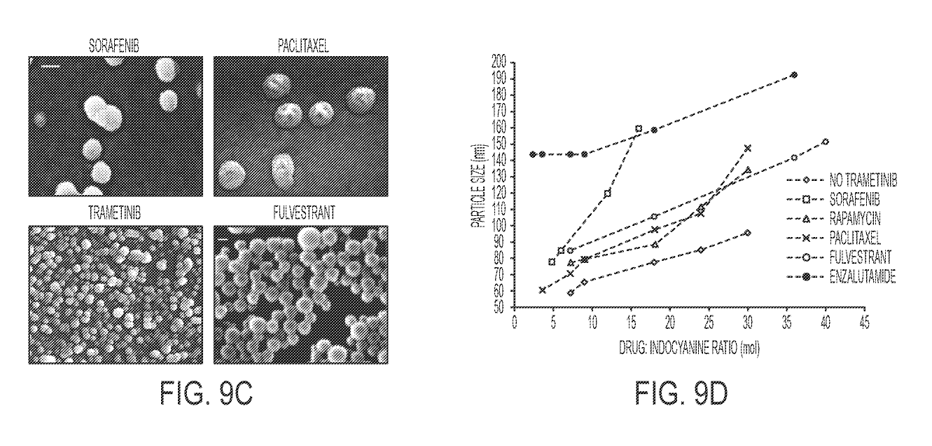

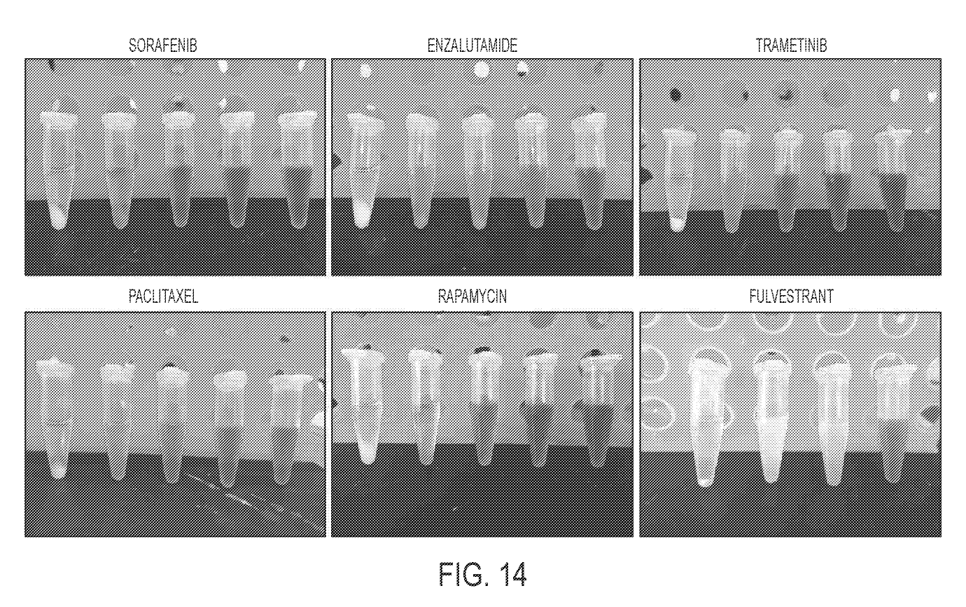

The self-assembly of multiple hydrophobic drugs with sulfated indocyanine compounds were investigated using the nano-precipitation methods described herein. It was found that the indocyanine compound IR783 stably suspended certain hydrophobic drugs in aqueous buffer (FIG. 9A, FIG. 14). Upon suspension of the drugs, it was noted that a distinct color change of the solutions occurred. Absorbance spectroscopy (FIG. 9B) revealed a relative increase of the λₘₐₓ=780 nm peak and a decrease of the λₘₐₓ=640 nm in the presence of drug, consistent with the dissolution of indocyanine H-aggregates. Without wishing to be bound to any theory, the 780 nm peak also exhibited a distinct shift towards longer wavelength values, suggesting the potential formation of J-aggregates by some of the indocyanine molecules in the presence of drug.

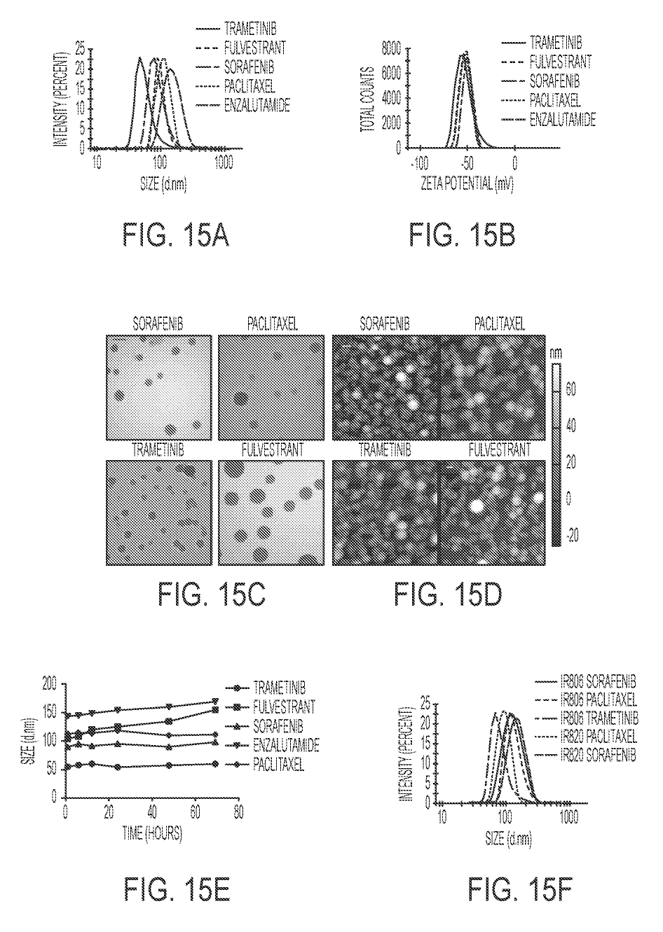

The resulting drug suspensions were characterized by DLS, SEM, AFM, and TEM, confirming that nanoparticles formed (FIG. 9C, FIGS. 15A-15F). The particle sizes ranged from 50 nm to 140 nm, dependent on the drug type and concentration (FIG. 9D). The drug loading within the indocyanine nanoparticles was remarkably high, reaching 90% for fulvestrant-based INPs, 84% for paclitaxel INPs, 82% for trametinib INPs, and 86% for sorafenib INPs. Additional characterization data is shown in FIGS. 15A-15F.

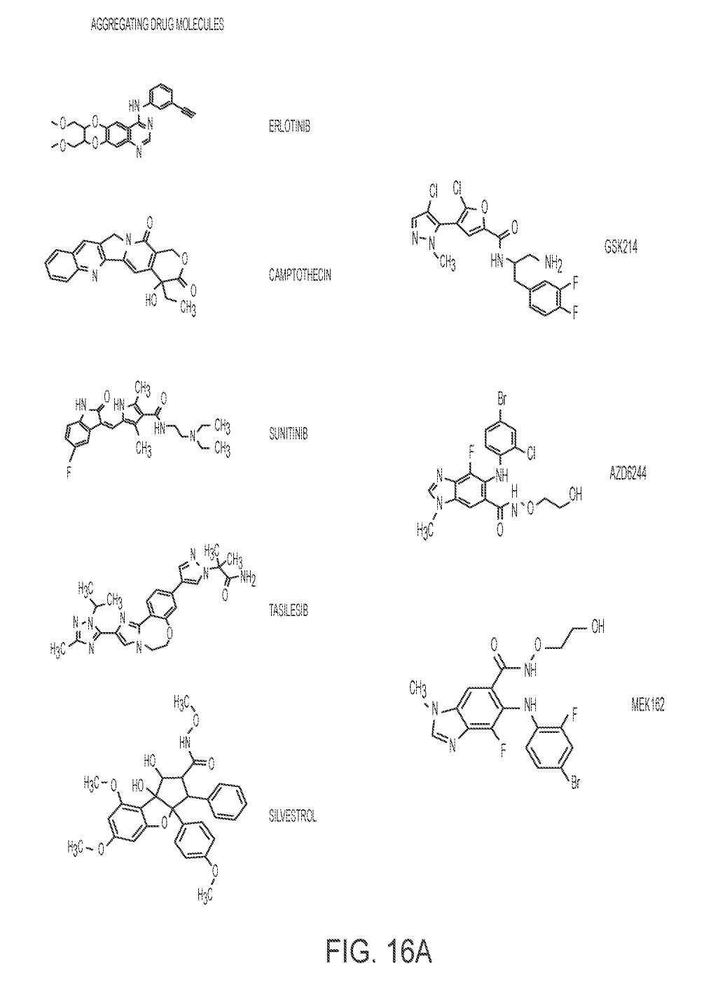

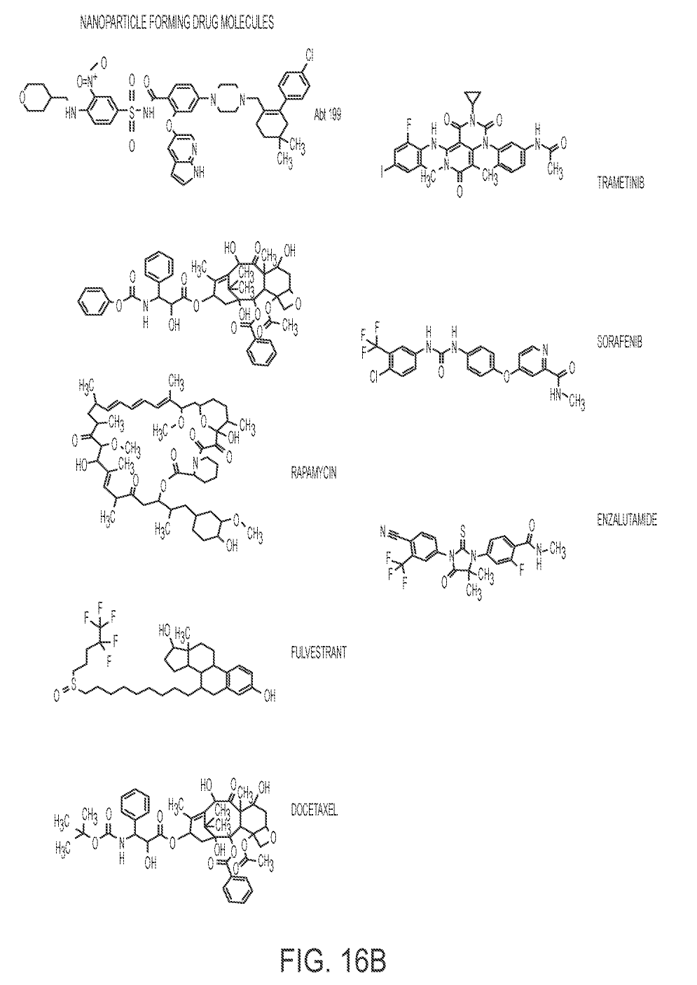

From 18 drug compounds assessed, 8 formed nanoparticles with indocyanine and 10 did not for nanoparticles with indocyanine. As the compounds were mostly similar in their molecular weight, hydrophobicity, and charge, a larger set of chemical properties were investigated to understand the factors mediating nanoparticle self-assembly. Quantitative structure-property relationship (QSPR) analysis was used to understand the self-assembly process and to potentially predict such assembly behavior and nanoparticle properties based on the molecular structures of the drugs. Molecular descriptors of drugs which correlated with the successful suspension via indocyanine into nanoparticles were searched. A training set of 16 hydrophobic drug molecules (FIGS. 16A and 16B) was built using a binary ranking to denote nanoparticle-forming and precipitating compounds, based on the observed stability of the suspensions (Table 1). DRAGON 6 software (Talette) was used to calculate 4886 descriptors from the molecular structures of the drugs in the training set which we assessed for correlations with the experimental data.

Table 1 shows drugs that were validated experimentally for formation of indocyanine nanoparticles.

TABLE 1

| | |
|---|---|
| 1 | ABT737 |
| 2 | Tacrolimus |
| 3 | BMS-777607 |
| 4 | Docetaxel |
| 5 | Tanespimycin |
| 6 | Everolimus |
| 7 | Trametinib |
| 8 | Vemurafenib |
| 9 | Nilotinib |
| 10 | Paclitaxel |
| 11 | Sorafenib |
| 12 | Navitoclax |
| 13 | Celecoxib |
| 14 | Avagacestat |
| 15 | Dutasteride |
| 16 | Enzalutamide |
| 17 | Fulvastrant |
| 18 | Regorafenib |
| 19 | RO4929097 |
| 20 | Valrubicin |

The Burden matrix is a topochemical index which scores molecules by their geometrical complexity, bond order, and heteroatoms. The intrinsic state of the ith atom Iᵢ is a local vertex invariant calculated from the molecular graph as the following:

$${l}_{i} = \frac{{( 2 \text{/} {L}_{i} )}^{2} \times {δ}_{i}^{v} + 1}{{δ}_{i}}$$

where L is the principal quantum number, δv is the number of valence electrons (valence vertex degree), and δ is the number of sigma electrons of the ith atom in the H-depleted molecular structure.

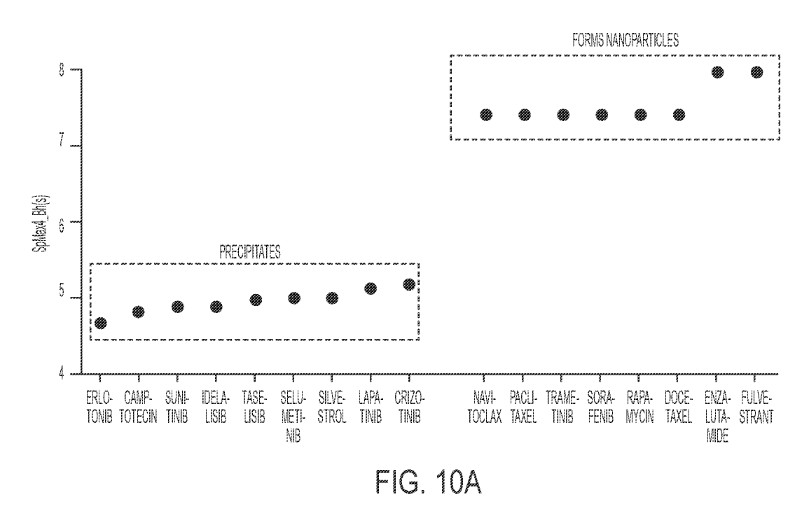

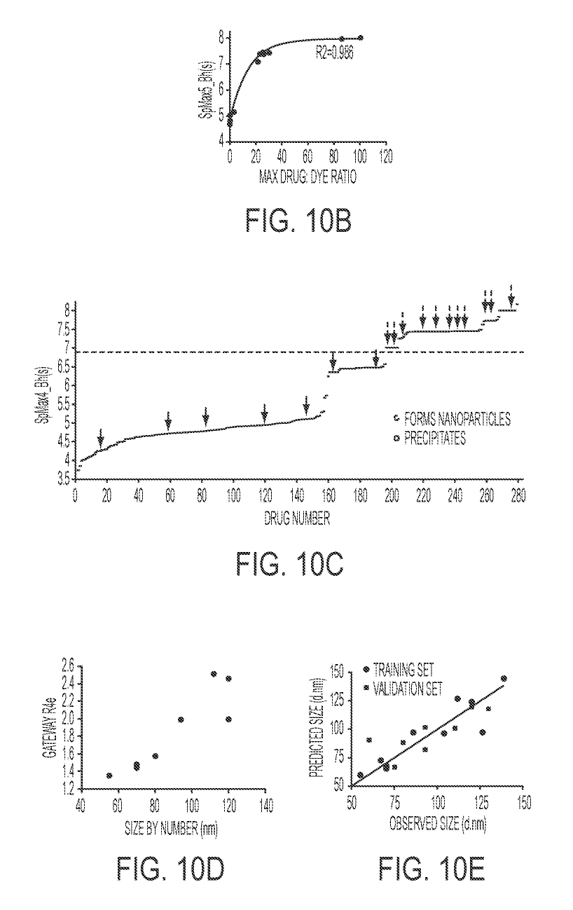

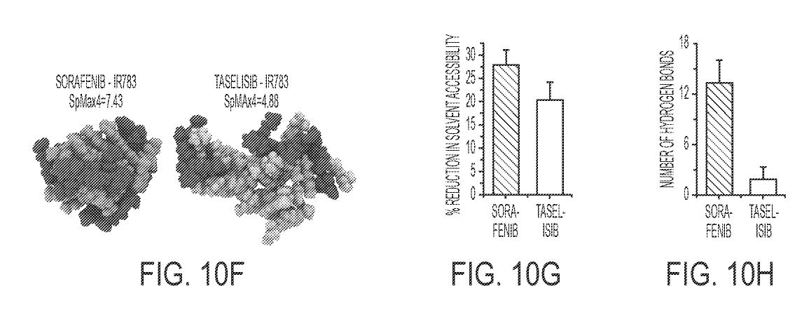

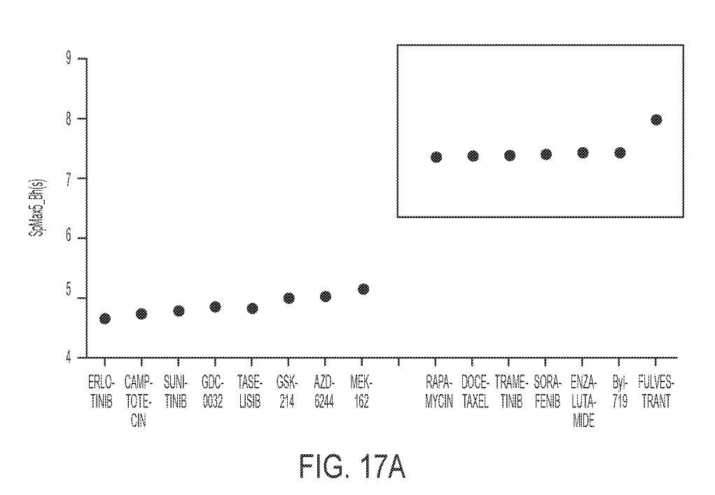

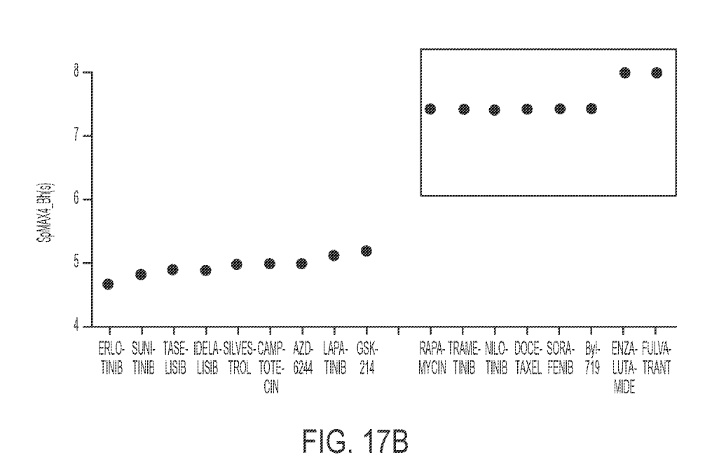

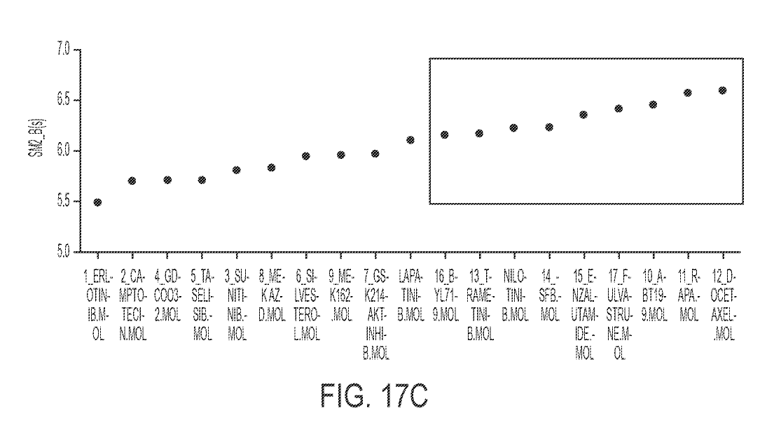

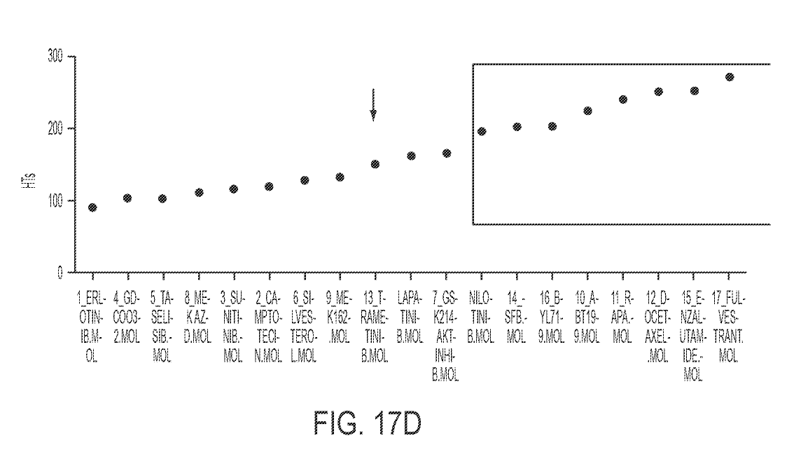

Surprisingly, the analysis identified four molecular descriptors that correlated highly with the experimental data set, giving correlation coefficients of over 0.85 (FIGS. 17A-17D). One descriptor, SpMAX4_Bh(s), or the leading eigenvalue of the Burden matrix weighted by the intrinsic state(s), gave a correlation coefficient of 0.98. The analysis showed that the calculated eigenvalues of the nanoparticle-forming drugs were above 7.3, while the non-assembling drug eigenvalues were between 4 and 5.5 (FIG. 10A). A similar descriptor, SpMAX5_Bh(s), correlated highly (coeff.=0.89) to experimental data corresponding to the maximal drug:indocyanine ratio which formed stable nanoparticles (FIG. 10B).

To assess the strength of the QSAP analysis via the SpMAX4_Bh(s) descriptor, the related eigenvalues of 280 insoluble drug molecules (less than 0.1 mg/ml solubility in water according to DrugBank database) with A Log P2 values (a molecular descriptor of hydrophobicity) of over 4.5 were calculated. Out of the analyzed molecules, 71 molecules were identified with eigenvalues of over 6.99 and A Log P2 values over 4.5, according to the training set data, should form nanoparticles (Table 2, Table 3). A validation set of 18 drug molecules with disparate SpMAX4_Bh(s) values was experimentally tested for nanoparticle formation with indocyanine. Notably, all drugs behaved as predicted; molecules with eigenvalues above 6.99 formed nanoparticles, while those under 6.99 precipitated (FIG. 10C).

Table 2 shows molecular descriptors calculated for 280 drugs. Molecular structures were minimized before calculations.

TABLE 2

| Drug No. | NAME | SpMax4_Bh(s) | SpMax5_Bh(s) | R4e | ALOGP2 |
|---|---|---|---|---|---|
| 1 | Thymol | 3.736 | 3.467 | 1.744 | 10.517 |
| 2 | Thiabendazole | 3.852 | 3.35 | 0.639 | 4.642 |
| 3 | Tizanidine | 3.983 | 3.872 | 0.961 | 6.174 |
| 4 | Tripelennamine | 4.016 | 3.823 | 1.544 | 9.628 |
| 5 | Thioridazine | 4.019 | 3.887 | 1.71 | 30.949 |
| 6 | Valproic Acid | 4.04 | 3.584 | 1.69 | 7.422 |
| 7 | Trimipramine | 4.068 | 3.929 | 1.886 | 23.393 |
| 8 | Tapentadol | 4.104 | 3.876 | 1.768 | 11.84 |
| 9 | Triprolidine | 4.108 | 3.826 | 1.476 | 16.606 |
| 10 | Trioxsalen | 4.132 | 3.964 | 1.143 | 10.768 |
| 11 | Tymazoline | 4.157 | 3.734 | 1.203 | 9.246 |
| 12 | Tavaborole | 4.216 | 3.582 | 1.199 | 4.628 |
| 13 | Thiopental | 4.251 | 4.028 | 2.091 | 7.885 |
| 14 | Chloroquine | 4.253 | 4.085 | 1.545 | 18.884 |
| 15 | Fluorouracil | 4.274 | 3.572 | 1.056 | 1.214 |
| 16 | Venlafaxine | 4.275 | 4.154 | 1.955 | 9.133 |
| 17 | Terbinafine | 4.286 | 4.116 | 1.891 | 28.47 |
| 18 | Tasimelteon | 4.291 | 4.077 | 1.333 | 8.44 |
| 19 | Tolnaftate | 4.336 | 3.967 | 1.623 | 32.147 |
| 20 | Abiraterone | 4.373 | 3.908 | 2.466 | 17.835 |
| 21 | Tolterodine | 4.387 | 4.048 | 1.638 | 31.769 |
| 22 | Tamoxifen | 4.399 | 4.233 | 1.351 | 39.939 |
| 23 | LDN-212854 | 4.418 | 4.354 | 1.531 | 10.82 |
| 24 | Trazodone | 4.424 | 4.294 | 1.696 | 5.851 |
| 25 | Sotradecol | 4.428 | 4.233 | 1.698 | 17.637 |
| 26 | Tazarotene | 4.475 | 4.371 | 1.763 | 24.518 |
| 27 | Triazolam | 4.488 | 4.324 | 1.227 | 19.28 |
| 28 | Verapamil | 4.496 | 4.496 | 1.712 | 30.636 |
| 29 | Tofisopam | 4.497 | 4.495 | 1.222 | 16.231 |
| 30 | Tretinoin | 4.51 | 4.302 | 1.893 | 30.532 |
| 31 | AZ 3146 | 4.566 | 4.344 | 1.921 | 11.671 |
| 32 | Thioproperazine | 4.569 | 4.406 | 2.037 | 13.5 |
| 33 | Thiothixene | 4.57 | 4.409 | 1.917 | 16.471 |
| 34 | Trimethobenzamide | 4.58 | 4.462 | 1.528 | 7.261 |
| 35 | Tiagabine | 4.583 | 4.324 | 1.713 | 27.688 |
| 36 | Anacardic Acid | 4.586 | 4.507 | 1.594 | 51.134 |
| 37 | voxtalisib | 4.621 | 4.166 | 0.961 | 0.703 |
| 38 | Astemizole | 4.624 | 4.431 | 1.607 | 32.375 |
| 39 | Sufentanil | 4.626 | 4.287 | 1.322 | 10.439 |
| 40 | Tioconazole | 4.636 | 4.486 | 1.216 | 19.693 |
| 41 | Fenretinide | 4.638 | 4.512 | 1.926 | 41.454 |
| 42 | Tegafur | 4.647 | 4.098 | 1.441 | 0.391 |
| 43 | AZD8055 | 4.648 | 4.622 | 1.596 | 10.72 |
| 44 | Bexarotene | 4.65 | 4.445 | 2.162 | 40.706 |
| 45 | Vandetanib | 4.65 | 4.483 | 1.86 | 25.882 |
| 46 | ML323 | 4.655 | 4.148 | 1.422 | 25.423 |
| 47 | Triclosan | 4.657 | 4.476 | 1.257 | 26.173 |
| 48 | Terazosin | 4.661 | 4.604 | 1.57 | 2.213 |
| 49 | Rhodamine B | 4.67 | 4.615 | 1.279 | 12.09 |
| 50 | erlotinib | 4.671 | 4.66 | 1.308 | 18.575 |
| 51 | Toremifene | 4.68 | 4.396 | 1.343 | 39.66 |
| 52 | Mibefradil | 4.689 | 4.598 | 1.742 | 31.216 |
| 53 | Trimetrexate | 4.689 | 4.58 | 1.263 | 7.752 |
| 54 | Axitinib | 4.7 | 4.372 | 1.18 | 20.186 |
| 55 | Thiamylal | 4.703 | 4.215 | 2.071 | 8.257 |
| 56 | Tolbutamide | 4.704 | 4.241 | 1.722 | 5.457 |
| 57 | Tolazamide | 4.705 | 4.371 | 1.925 | 6.231 |
| 58 | Masitinib | 4.711 | 4.654 | 1.786 | 20.029 |
| 59 | Suprofen | 4.711 | 4.612 | 1.491 | 10.819 |
| 60 | Tolfenamic Acid | 4.713 | 4.353 | 1.428 | 17.099 |
| 61 | Tenovin-1 | 4.714 | 4.674 | 1.952 | 14.551 |
| 62 | TAE226 | 4.717 | 4.615 | 1.419 | 15.348 |
| 63 | Risperidone | 4.718 | 4.318 | 1.855 | 11.006 |
| 64 | AZD4547 | 4.722 | 4.58 | 1.855 | 17.677 |
| 65 | Amuvatinib | 4.723 | 4.36 | 1.578 | 17.206 |
| 66 | Tiaprofenic acid | 4.735 | 4.587 | 1.544 | 12.821 |
| 67 | CP-724714 | 4.736 | 4.609 | 1.569 | 17.647 |
| 68 | MK2206 | 4.737 | 4.67 | 1.47 | 11.695 |
| 69 | INK128 | 4.748 | 4.51 | 1.194 | 3.051 |
| 70 | Prasugrel | 4.749 | 4.638 | 1.404 | 14.201 |
| 71 | duvelisib | 4.754 | 4.579 | 1.36 | 12.311 |
| 72 | Degrasyn | 4.755 | 4.208 | 1.269 | 20.67 |
| 73 | Gefitinib | 4.755 | 4.626 | 1.54 | 17.668 |
| 74 | Tolmetin | 4.755 | 4.674 | 1.504 | 10.511 |
| 75 | sepantronium | 4.756 | 4.648 | 1.329 | 2.447 |
| | bromide | | | | |
| 76 | Panobinostat | 4.761 | 4.338 | 1.558 | 7.979 |
| 77 | Terconazole | 4.761 | 4.612 | 1.851 | 24.502 |
| 78 | Tamibarotene | 4.764 | 4.65 | 2.341 | 21.607 |
| 79 | Temazepam | 4.771 | 4.441 | 1.263 | 9.273 |
| 80 | Suvorexant | 4.779 | 4.631 | 1.989 | 16.876 |
| 81 | Avasimibe | 4.786 | 4.698 | 1.89 | 67.695 |
| 82 | SGI-1027 | 4.79 | 4.709 | 1.357 | 22.265 |
| 83 | Adapalene | 4.792 | 4.428 | 2.179 | 39.118 |
| 84 | Flurbiprofen | 4.792 | 4.539 | 1.579 | 13.363 |
| 85 | IWP-L6 | 4.792 | 4.684 | 1.179 | 18.366 |
| 86 | Tasosartan | 4.792 | 4.58 | 1.471 | 9.312 |
| 87 | Sulfinpyrazone | 4.794 | 4.72 | 1.333 | 11.792 |
| 88 | Linifanib | 4.796 | 4.688 | 1.518 | 17.383 |
| 89 | Trifluoperazine | 4.804 | 4.571 | 1.959 | 24.752 |
| 90 | Triflupromazine | 4.804 | 4.571 | 1.806 | 25.178 |
| 91 | Torkinib | 4.811 | 4.494 | 1.288 | 7.527 |
| 92 | sunitinib | 4.819 | 4.646 | 1.464 | 8.987 |
| 93 | Atenolol | 4.822 | 4.369 | 1.75 | 0.448 |
| 94 | Idelalisib | 4.828 | 4.695 | 1.249 | 11.907 |
| 95 | Valdecoxib | 4.828 | 4.714 | 1.322 | 7.415 |
| 96 | CEP-28122 | 4.832 | 4.627 | 2.037 | 16.529 |
| 97 | PD0332991 | 4.838 | 4.392 | 1.566 | 7.081 |
| 98 | Afatinib | 4.858 | 4.747 | 1.499 | 15.346 |
| 99 | Tamsulosin | 4.858 | 4.77 | 1.44 | 8.082 |
| 100 | Ketoconazole | 4.865 | 4.758 | 1.706 | 13.035 |
| 101 | Itraconazole | 4.875 | 4.8 | 1.883 | 41.82 |
| 102 | GDC0032 | 4.887 | 4.844 | 1.735 | 6.101 |
| 103 | SB-431542 | 4.896 | 4.793 | 1.048 | 11.883 |
| 104 | Tivantinib | 4.898 | 4.312 | 1.637 | 12.397 |
| 105 | Cinacalcet | 4.9 | 4.693 | 1.594 | 35.483 |
| 106 | MGCD-265 | 4.901 | 4.865 | 1.555 | 30.974 |
| 107 | XL888 | 4.903 | 4.86 | 1.745 | 14.233 |
| 108 | iloperidone | 4.908 | 4.472 | 1.727 | 16.133 |
| 109 | Voacamine | 4.917 | 4.623 | 2.23 | 56.953 |
| 110 | ABT-751 | 4.92 | 4.844 | 1.62 | 11.867 |
| 111 | Tolvaptan | 4.923 | 4.743 | 1.831 | 26.289 |
| 112 | Tariquidar | 4.927 | 4.904 | 1.619 | 31.059 |
| 113 | Dactolisib | 4.931 | 4.718 | 1.808 | 28.788 |
| 114 | Crizotinib | 4.932 | 4.616 | 1.746 | 13.646 |
| 115 | Ipatasertib | 4.934 | 4.412 | 1.947 | 10.296 |
| 116 | Fluorescein | 4.937 | 4.69 | 1.564 | 13.588 |
| 117 | Tadalafil | 4.937 | 4.405 | 1.714 | 6.814 |
| 118 | Telmisartan | 4.943 | 4.793 | 1.603 | 60.44 |
| 119 | Vilazodone | 4.946 | 4.857 | 1.706 | 20.577 |
| 120 | Udenafil | 4.947 | 4.802 | 1.681 | 15.725 |
| 121 | Neratinib | 4.96 | 4.872 | 1.466 | 25.957 |
| 122 | CUDC-907 | 4.966 | 4.905 | 1.563 | 4.612 |
| 123 | silvesterol | 4.971 | 4.85 | 1.372 | 5.653 |
| 124 | Pictilisib | 4.975 | 4.612 | 1.685 | 8.361 |
| 125 | Sulfaphenazole | 4.99 | 4.658 | 1.601 | 5.734 |
| 126 | Camptothecin | 4.993 | 4.571 | 1.885 | 3.049 |
| 127 | Selumetinib | 4.996 | 4.923 | 1.115 | 10.461 |
| 128 | Rocilinostat | 4.997 | 4.763 | 1.445 | 12.59 |
| 129 | IWR-1-endo | 4.998 | 4.7 | 1.675 | 7.066 |
| 130 | vismodegib | 4.998 | 4.884 | 1.578 | 18.197 |
| 131 | GSK923295 | 5.049 | 4.971 | 1.872 | 22.848 |
| 132 | pantoprazole | 5.053 | 4.981 | 1.274 | 8.71 |
| 133 | Alisertib | 5.058 | 4.896 | 1.359 | 35.153 |
| 134 | Talazoparib | 5.069 | 4.794 | 1.525 | 6.088 |
| 135 | Cabozantinib | 5.08 | 4.834 | 1.644 | 18.86 |
| 136 | Roflumilast | 5.083 | 4.868 | 1.223 | 19.379 |
| 137 | K-252a | 5.084 | 4.809 | 1.782 | 12.648 |
| 138 | Fluconazole | 5.088 | 5.064 | 1.711 | 0.563 |
| 139 | Sulfadimethoxine | 5.09 | 4.596 | 1.386 | 3.353 |
| 140 | GDC0810 | 5.098 | 4.895 | 1.284 | 46.328 |
| 141 | CUDC-101 | 5.1 | 4.767 | 1.473 | 25.069 |
| 142 | Sulfamoxole | 5.103 | 4.441 | 1.634 | 0.829 |
| 143 | Barasertib | 5.109 | 4.966 | 1.523 | 14.22 |
| 144 | Lapatinib | 5.124 | 5.008 | 1.578 | 39.126 |
| 145 | Fragment | 5.17 | 4.964 | 1.336 | 5.486 |
| 146 | Mubritinib | 5.171 | 5.109 | 1.759 | 36.696 |
| 147 | Luminespib | 5.188 | 4.714 | 1.772 | 16.148 |
| 148 | Valsartan | 5.268 | 4.798 | 1.691 | 21.347 |
| 149 | ZM 336372 | 5.719 | 5.464 | 0.755 | 14.22 |
| 150 | Bortezomib | 6.23 | 4.975 | 1.554 | 3.683 |
| 151 | celastrol | 6.351 | 4.695 | 3.037 | 27.926 |
| 152 | grepafloxacin | 6.351 | 4.657 | 1.69 | 5.17 |
| 153 | Levocabastine | 6.351 | 4.752 | 2.209 | 26.129 |
| 154 | Pob-SAM | 6.351 | 5.439 | 1.71 | 0.092 |
| 155 | Sulindac | 6.351 | 4.806 | 1.218 | 13.06 |
| 156 | Trandolapril | 6.351 | 4.753 | 1.778 | 14.168 |
| 157 | Treprostinil | 6.351 | 4.82 | 2.025 | 21.376 |
| 158 | Ursodeoxycholic | 6.351 | 4.465 | 2.837 | 15.915 |
| | acid | | | | |
| 159 | Teniposide | 6.401 | 5.011 | 1.554 | 6.18 |
| 160 | Fludarabine | 6.437 | 6.4 | 1.826 | 5.115 |
| 161 | Pitavastatin | 6.437 | 6.35 | 1.423 | 15.434 |
| 162 | Halotestin | 6.442 | 4.439 | 3.111 | 6.239 |
| 163 | Formebolone | 6.443 | 4.521 | 2.889 | 3.833 |
| 164 | bafilomycin a | 6.445 | 6.433 | 2.468 | 22.562 |
| 165 | Epothilone A | 6.445 | 4.863 | 2.373 | 11.795 |
| 166 | KW-2478 | 6.445 | 4.911 | 1.628 | 6.877 |
| 167 | digitoxin | 6.446 | 6.443 | 2.751 | 9.618 |
| 168 | Voriconazole | 6.446 | 5.077 | 1.635 | 4.293 |
| 169 | Vinblastine | 6.447 | 6.439 | 2.334 | 23.886 |
| 170 | Delanzomib | 6.45 | 6.23 | 1.67 | 8.703 |
| 171 | Dexamethasone | 6.456 | 6.445 | 2.857 | 2.696 |
| 172 | Permitil | 6.456 | 4.804 | 1.793 | 19.675 |
| 173 | Ticagrelor | 6.456 | 6.4 | 1.443 | 7.309 |
| 174 | MEK162 | 6.457 | 4.982 | 1.28 | 7.703 |
| 175 | Atorvastatin | 6.462 | 6.436 | 1.41 | 30.588 |
| 176 | Gemcitabine | 6.464 | 6.436 | 1.994 | 2.03 |
| 177 | Tafluprost | 6.464 | 6.434 | 1.615 | 18.74 |
| 178 | Dantron | 6.465 | 4.61 | 1.467 | 5.169 |
| 179 | Trabectedin | 6.466 | 6.449 | 2.156 | 20.392 |
| 180 | Tenoxicam | 6.468 | 4.906 | 1.766 | 3.578 |
| 181 | AS-252424 | 6.469 | 5.059 | 1.037 | 9.52 |
| 182 | Sulfasalazine | 6.469 | 6.35 | 1.51 | 15.093 |
| 183 | Besifloxacin | 6.471 | 6.449 | 1.518 | 21.231 |
| 184 | ezetimibe | 6.471 | 6.449 | 1.518 | 21.231 |
| 185 | ICG-001 | 6.471 | 4.898 | 1.992 | 17.197 |
| 186 | Nystatin | 6.471 | 6.459 | 2.651 | 0.079 |
| 187 | Doxorubicin | 6.481 | 6.454 | 2.036 | 0 |
| 188 | NMS-E973 | 6.493 | 6.446 | 2.021 | 7.624 |
| 189 | AT101 Glossypol | 6.523 | 6.472 | 1.879 | 41.479 |
| 190 | TW-37 | 6.56 | 6.445 | 2.165 | 56.015 |
| 191 | ABT737 | 6.999 | 6.999 | 1.871 | 67.217 |
| 192 | Argatroban | 6.999 | 6.351 | 2.091 | 2.516 |
| 193 | C646 | 6.999 | 6.351 | 1.419 | 20.894 |
| 194 | PHA-665752 | 6.999 | 5.005 | 1.932 | 32.372 |
| 195 | Remikiren | 6.999 | 6.493 | 2.185 | 12.065 |
| 196 | SU11274 | 6.999 | 4.941 | 1.898 | 16.787 |
| 197 | Venetoclax | 6.999 | 6.999 | 2.052 | 60.859 |
| 198 | XL388 | 6.999 | 5.176 | 1.721 | 9.046 |
| 199 | Encorafenib | 6.999 | 5.055 | 1.57 | 16.954 |
| 200 | RO5126766 | 6.999 | 5.1 | 1.733 | 9.863 |
| 201 | AMI-1 | 7.246 | 7.243 | 1.001 | 0 |
| 202 | Tacrolimus | 7.352 | 6.453 | 2.31 | 21.703 |
| 203 | Lansoprazole | 7.397 | 5.043 | 1.297 | 13.365 |
| 204 | AS-604850 | 7.409 | 4.915 | 1.019 | 14.023 |
| 205 | BMS-777607 | 7.413 | 5.224 | 1.43 | 10.961 |
| 206 | Foretinib | 7.413 | 5.071 | 1.769 | 19.688 |
| 207 | Telithromycin | 7.413 | 6.449 | 2.221 | 15.148 |
| 208 | Alvespimycin | 7.418 | 6.449 | 1.996 | 3.12 |
| 209 | Geldanamycin | 7.418 | 6.449 | 1.96 | 3.587 |
| 210 | Tanespimycin | 7.418 | 6.449 | 1.883 | 5.038 |
| 211 | Latamoxef | 7.421 | 6.471 | 1.644 | 0.106 |
| 212 | Oprozomib | 7.421 | 4.818 | 1.615 | 0 |
| 213 | Carfilzomib | 7.423 | 7.421 | 1.747 | 18.487 |
| 214 | Cefapirin | 7.423 | 6.351 | 1.462 | 0.002 |
| 215 | Floxapen | 7.423 | 6.351 | 1.878 | 7.256 |
| 216 | Everolimus | 7.424 | 7.352 | 2.21 | 35.5 |
| 217 | Fludrocortisone | 7.424 | 6.449 | 2.69 | 2.975 |
| 218 | Rapamycin | 7.424 | 7.352 | 2.441 | 37.077 |
| 219 | Sonolisib | 7.424 | 6.459 | 1.771 | 2.465 |
| 220 | Wortmannin | 7.424 | 5.012 | 2.117 | 2.451 |
| 221 | Zotarolimus | 7.424 | 7.352 | 2.395 | 35.24 |
| 222 | Balifomycin f | 7.425 | 6.464 | 2.488 | 21.876 |
| 223 | Olaparib | 7.425 | 4.835 | 1.694 | 4.525 |
| 224 | Telaprevir | 7.425 | 7.421 | 1.808 | 8.833 |
| 225 | Temsirolimus | 7.425 | 7.424 | 2.496 | 34.025 |
| 226 | Anidulafungin | 7.426 | 7.424 | 2.196 | 0.066 |
| 227 | Cromolyn | 7.426 | 6.45 | 1.429 | 2.959 |
| 228 | Lubiprostone | 7.426 | 6.448 | 2.218 | 13.564 |
| 229 | LY411575 | 7.426 | 7.422 | 1.716 | 8.675 |
| 230 | Trametinib | 7.426 | 7.411 | 1.351 | 10.091 |
| 231 | Cobimetinib | 7.427 | 6.447 | 1.909 | 13.702 |
| 232 | Vemurafenib | 7.427 | 6.999 | 1.442 | 27.945 |
| 233 | Verteporfin | 7.428 | 6.351 | 1.868 | 60.28 |
| 234 | Viomycin | 7.428 | 7.425 | 2.213 | 69.421 |
| 235 | Nilotinib | 7.429 | 5.05 | 1.482 | 25.849 |
| 236 | OSI-930 | 7.429 | 4.994 | 1.372 | 39.269 |
| 237 | PND-1186 | 7.429 | 4.941 | 1.352 | 18.512 |
| 238 | Fluticasone | 7.43 | 7.426 | 2.817 | 8.415 |
| 239 | Troleandomycin | 7.43 | 7.424 | 2.1 | 8.405 |
| 240 | Vincristine | 7.43 | 6.447 | 2.271 | 16.035 |
| 241 | Florone | 7.431 | 7.428 | 2.208 | 5.009 |
| 242 | Paclitaxel | 7.431 | 7.427 | 1.995 | 9.789 |
| 243 | Docetaxel | 7.431 | 7.426 | 2.516 | 7.075 |
| 244 | Sorafenib | 7.432 | 7.428 | 1.575 | 17.437 |
| 245 | PF-03814735 | 7.434 | 7.423 | 1.498 | 10.476 |
| 246 | Travoprost | 7.434 | 7.248 | 0.501 | NaN |
| 247 | Efavirenz | 7.435 | 4.957 | 1.226 | 19.201 |
| 248 | PF-04929113 | 7.439 | 7.432 | 1.963 | 11.155 |
| 249 | Valrubicin | 7.441 | 7.432 | 1.898 | 9.283 |
| 250 | MK-0752 | 7.474 | 6.999 | 1.922 | 31.253 |
| 251 | GSK2636771 | 7.475 | 6.351 | 1.716 | 18.306 |
| 252 | Trovafloxacin | 7.611 | 7.425 | 0.701 | NaN |
| 253 | Saprisartan | 7.703 | 7.437 | 1.306 | 51.121 |
| 254 | Defactinib | 7.716 | 7.429 | 1.613 | 6.799 |
| 255 | PF-431396 | 7.716 | 7.429 | 1.547 | 13.857 |
| 256 | PF-00562271 | 7.716 | 7.429 | 1.572 | 9.678 |
| 257 | PF-573228 | 7.716 | 7.429 | 1.585 | 15.743 |
| 258 | Tipranavir | 7.717 | 7.433 | 1.909 | 54.698 |
| 259 | Navitoclax | 7.719 | 7.7 | 2.072 | 110.149 |
| 260 | Celecoxib | 7.726 | 6.999 | 1.381 | 20.716 |
| 261 | Dabrafenib | 7.716 | 6.999 | 5.234 | 1.816 |
| 262 | Andarine | 7.815 | 7.432 | 1.961 | 5.358 |
| 263 | Flutamide | 7.815 | 7.428 | 1.732 | 8.506 |
| 264 | Nilutamide | 7.815 | 7.447 | 2.256 | 5.085 |
| 265 | Evans Blue | 7.854 | 6.999 | 2.115 | 11.335 |
| 266 | antrafenine | 7.999 | 7.999 | 1.801 | 50.049 |
| 267 | Aprepitant | 7.999 | 7.999 | 1.773 | 23.513 |
| 268 | Avagacestat | 7.999 | 7.715 | 1.613 | 15.962 |
| 269 | Bicalutamide | 7.999 | 7.714 | 1.991 | 8.566 |
| 270 | Dutasteride | 7.999 | 7.999 | 2.629 | 32.536 |
| 271 | Enzalutamide | 7.999 | 7.43 | 1.998 | 18.789 |
| 272 | Fulvastrune | 7.999 | 7.999 | 2.464 | 70.768 |
| 273 | Lomitapide | 7.999 | 7.999 | 1.797 | 60.697 |
| 274 | Mefloquine | 7.999 | 7.999 | 1.912 | 18.51 |
| 275 | Regorafenib | 7.999 | 7.432 | 1.65 | 19.196 |
| 276 | RO4929097 | 7.999 | 7.999 | 1.838 | 10.833 |
| 277 | TAK-632 | 7.999 | 7.43 | 7.426 | 1.635 |
| 278 | Sitagliptin | 8.161 | 7.999 | 2.06 | 4.596 |

Table 3 shows molecular descriptors calculated for 71 molecules that were identified with eigenvalues of over 6.99 and A Log P2 values over 4.5, according to the training set data, should form nanoparticles. Molecular structures were minimized before calculations. The molecular structures for each of the drugs are listed in Table 5.

TABLE 3

| Drug | | | | | |
|---|---|---|---|---|---|
| No. | NAME | SpMax4_Bh(s) | R4e | ALOGP2 | IUPAC |
| 1 | XL388 | 6.999 | 1.721 | 9.046 | (7-(6-Amino-3- |
| | | | | | pyridinyl)-2,3-dihydro- |
| | | | | | 1,4-benzoxazepin-4(5H)- |
| | | | | | yl)(3-fluoro-2-methyl-4- |
| | | | | | (methylsulfonyl)phenyl)- |
| | | | | | methanone |
| 2 | RO5126766 | 6.999 | 1.733 | 9.863 | N-(3-Fluoro-4-{[4- |
| | | | | | methyl-2-oxo-7-(2- |
| | | | | | pyrimidinyloxy)-2H- |
| | | | | | chromen-3-yl]methyl}-2- |
| | | | | | pyridinyl)-N′- |
| | | | | | methylsulfuric diamide |
| 3 | Remikiren | 6.999 | 2.185 | 12.065 | Nα-[(2S)-2-Benzyl-3- |
| | | | | | (tert- |
| | | | | | butylsulfonyl)propanoyl]- |
| | | | | | N-[(2S,3R,4S)-1- |
| | | | | | cyclohexyl-4- |
| | | | | | cyclopropyl-3,4- |
| | | | | | dihydroxybutan-2-yl]-L- |
| | | | | | histidinamide |
| 4 | SU11274 | 6.999 | 1.898 | 16.787 | (3Z) - N-(3- |
| | | | | | Chlorophenyl)-3-({3,5- |
| | | | | | dimethyl-4-[(4-methyl-1- |
| | | | | | piperazinyl)carbonyl]- |
| | | | | | 1H-pyrrol-2- |
| | | | | | yl}methylene)-N-methyl- |
| | | | | | 2-oxo-5- |
| | | | | | indolinesulfonamide |
| 5 | Encorafenib | 6.999 | 1.57 | 16.954 | Methyl [(2S)-1-{[4-(3- |
| | | | | | {5-chloro-2-fluoro-3- |
| | | | | | [(methylsulfonyl)amino]phenyl}- |
| | | | | | 1-isopropyl-1H- |
| | | | | | pyrazol-4-yl)-2- |
| | | | | | pyrimidinyl]amino}-2- |
| | | | | | propanyl]carbamate |
| 6 | C646 | 6.999 | 1.419 | 20.894 | 4-[(4Z)-4-{[5-(4,5- |
| | | | | | Dimethyl-2-nitrophenyl)- |
| | | | | | 2-furyl]methylene}-3- |
| | | | | | methyl-5-oxo-4,5- |
| | | | | | dihydro-1H-pyrazol-1- |
| | | | | | yl]benzoic acid |
| 7 | PHA-665752 | 6.999 | 1.932 | 32.372 | (3Z)-5-[(2,6- |
| | | | | | Dichlorobenzyl)sulfonyl]- |
| | | | | | 3-[(3,5-dimethyl-4- |
| | | | | | {[(2R)-2-(1- |
| | | | | | pyrrolidinylmethyl)-1- |
| | | | | | pyrrolidinyl]carbonyl}- |
| | | | | | 1H-pyrrol-2- |
| | | | | | yl)methylene]-1,3- |
| | | | | | dihydro-2H-indol-2-one |
| 8 | Venetoclaxl | 6.999 | 2.052 | 60.859 | 4-(4-{[2-(4- |
| | | | | | Chlorophenyl)-4,4- |
| | | | | | dimethyl-1-cyclohexen- |
| | | | | | 1-yl]methyl}-1- |
| | | | | | piperazinyl)-N-({3-nitro- |
| | | | | | 4-[(tetrahydro-2H-pyran- |
| | | | | | 4- |
| | | | | | ylmethyl)amino]phenyl}sulfonyl)- |
| | | | | | 2-(1H- |
| | | | | | pyrrolo[2,3-b]pyridin-5- |
| | | | | | yloxy)benzamide |
| 9 | ABT737 | 6.999 | 1.871 | 67.217 | 4-{4-[(4′-Chloro-2- |
| | | | | | biphenylyl)methyl]-1- |
| | | | | | piperazinyl}-N-[(4- |
| | | | | | {[(2R)-4- |
| | | | | | (dimethylamino)-1- |
| | | | | | (phenylsulfanyl)-2- |
| | | | | | butanyl]amino}-3- |
| | | | | | nitrophenyl)sulfonyl]benzamide |
| 10 | Tacrolimus | 7.352 | 2.31 | 21.703 | (1R,9S,12S,13R,14S,17R, |
| | | | | | 18E,21S,23S,24R,25S,27R)- |
| | | | | | 1,14-dihydroxy-12- |
| | | | | | {(E)-2-[(1R,3R,4R)-4- |
| | | | | | hydroxy-3- |
| | | | | | methoxycyclohexyl]-1- |
| | | | | | methylethenyl}-23,25- |
| | | | | | dimethoxy-13,19,21,27- |
| | | | | | tetramethyl-17-prop-2- |
| | | | | | en-1-yl-11,28-dioxa-4- |
| | | | | | azatricyclo[22.3.1.0^{4,9}]oc |
| | | | | | tacos-18-ene-2,3,10,16- |
| | | | | | tetrone |
| 11 | Lansoprazole | 7.397 | 1.297 | 13.365 | 2-({[3-Methyl-4-(2,2,2- |
| | | | | | trifluoroethoxy)-2- |
| | | | | | pyridinyl]methyl}sulfinyl)- |
| | | | | | 1H-benzimidazole |
| 12 | AS-604850 | 7.409 | 1.019 | 14.023 | (5E)-5-[(5-Benzyl-2,2- |
| | | | | | difluoro-1,3-dioxol-4- |
| | | | | | yl)methylene]-1,3- |
| | | | | | thiazolidine-2,4-dione |
| 13 | BMS-777607 | 7.413 | 1.43 | 10.961 | N-{4-[(2-Amino-3- |
| | | | | | chloro-4-pyridinyl)oxy]- |
| | | | | | 3-fluorophenyl}-4- |
| | | | | | ethoxy-1-(4- |
| | | | | | fluorophenyl)-2-oxo- |
| | | | | | 1,2,3,4-tetrahydro-3- |
| | | | | | pyridinecarboxamide |
| 14 | Telithromycin | 7.413 | 2.221 | 15.148 | (3aS,4R,7R,9R,10R,11R, |
| | | | | | 13R,15R,15aR)-10- |
| | | | | | {[(2S,3R,4S,6R)-4- |
| | | | | | (dimethylamino)-3- |
| | | | | | hydroxy-6- |
| | | | | | methyltetrahydro-2H- |
| | | | | | pyran-2-yl]oxy}-4-ethyl- |
| | | | | | 11-methoxy- |
| | | | | | 3a,7,9,11,13,15- |
| | | | | | hexamethyl-1-[4-(4- |
| | | | | | pyridin-3-yl-1H- |
| | | | | | imidazol-1- |
| | | | | | yl)butyl]octahydro-2H- |
| | | | | | oxacyclotetradecino[4,3- |
| | | | | | d][1,3]oxazole- |
| | | | | | 2,6,8,14(1H,7H,9H)- |
| | | | | | tetrone |
| 15 | Foretinib | 7.413 | 1.769 | 19.688 | N-[3-Fluoro-4-({6- |
| | | | | | methoxy-7-[3-(4- |
| | | | | | morpholinyl)propoxy]-4- |
| | | | | | quinoleinyl}oxy)phenyl]- |
| | | | | | N′-(4-fluorophenyl)-1,1- |
| | | | | | cyclopropanedicarboxamide |
| 16 | Floxapen | 7.423 | 1.878 | 7.256 | 4-Thia-1- |
| | | | | | azabicyclo[3.2.0]heptane- |
| | | | | | 2-carboxylic acid, 6- |
| | | | | | [[[3-(2-chloro-6- |
| | | | | | fluorophenyl)-5-methyl- |
| | | | | | 4- |
| | | | | | isoxazolyl]carbonyl]amino]- |
| | | | | | 3,3-dimethyl-7-oxo-, |
| | | | | | (2S,5R,6R)- |
| 17 | Carfilzomib | 7.423 | 1.747 | 18.487 | N-{(2S)-2-[(4- |
| | | | | | Morpholinylacetyl)amino]- |
| | | | | | 4-phenylbutanoyl}-L- |
| | | | | | leucyl-N-{(2S)-4- |
| | | | | | methyl-1-[(2R)-2- |
| | | | | | methyl-2-oxiranyl]-1- |
| | | | | | oxo-2-pentanyl}-L- |
| | | | | | phenylalaninamid |
| 18 | Zotarolimus | 7.424 | 2.395 | 35.24 | (1R,9S,12S,15R,16E,18R, |
| | | | | | 19R,21R,23S,24E,26E, |
| | | | | | 28E,30S,32S,35R)-1,18- |
| | | | | | Dihydroxy-19,30- |
| | | | | | dimethoxy-12-{(2R)-1- |
| | | | | | [(1S,3R,4S)-3-methoxy- |
| | | | | | 4-(1H-tetrazol-1- |
| | | | | | yl)cyclohexyl]-2- |
| | | | | | propanyl}- |
| | | | | | 15,17,21,23,29,35- |
| | | | | | hexamethyl-11,;36- |
| | | | | | dioxa-4- |
| | | | | | azatricyclo[30.3.1.0^{4,9}]hexatriaconta- |
| | | | | | 16,24,26,28- |
| | | | | | tetraene-2,3,10,14,20- |
| | | | | | pentone |
| 19 | Everolimus | 7.424 | 2.21 | 35.5 | (1R,9S,12S,15R,16E,18R, |
| | | | | | 19R,21R,23S,24E,26E, |
| | | | | | 28E,30S,35R)-1,18- |
| | | | | | Dihydroxy-12-{(2R)-1- |
| | | | | | [(1S,3R,4R)-4-(2- |
| | | | | | hydroxyethoxy)-3- |
| | | | | | methoxycyclohexyl]-2- |
| | | | | | propanyl}-19,30- |
| | | | | | dimethoxy- |
| | | | | | 15,17,21,23,29,35- |
| | | | | | hexamethyl-11,36- |
| | | | | | di;oxa-4- |
| | | | | | azatricyclo[30.3.1.0^{4,9}]hexatriaconta- |
| | | | | | 16,24,26,28- |
| | | | | | tetraene-2,3,10,14,20- |
| | | | | | pentone |
| 20 | Rapamycin | 7.424 | 2.441 | 37.077 | (1R,9S,12S,15R,16E,18R, |
| | | | | | 19R,21R,23S,24E,26E, |
| | | | | | 28E,30S,32S,35R)-1,18- |
| | | | | | dihydroxy-12-{(1R)-2- |
| | | | | | [(1S,3R,4R)-4-hydroxy- |
| | | | | | 3-methoxycyclohexyl]-1- |
| | | | | | methylethyl}-19,30- |
| | | | | | dimethoxy- |
| | | | | | 15,17,21,23,29,35- |
| | | | | | hexamethyl-11,36-dioxa- |
| | | | | | 4- |
| | | | | | azatricyclo[30.3.1.0^{4,9}]hexatriaconta- |
| | | | | | 16,24,26,28- |
| | | | | | tetraene-2,3,10,14,20- |
| | | | | | pentone |
| 21 | Telaprevir | 7.425 | 1.808 | 8.833 | (1S,3aR,6aS) - (2S)-2- |
| | | | | | cyclohexyl-N- |
| | | | | | (pyrazinylcarbonyl)glycyl- |
| | | | | | 3-methyl-L-valyl-N- |
| | | | | | ((1S)-1- |
| | | | | | ((cyclopropylamino)oxoacetyl)butyl)octahydrocyclopenta(c)pyrrole- |
| | | | | | 1- |
| | | | | | carboxamide |
| 22 | Balifomycin | 7.425 | 2.488 | 21.876 | (5R)-3-O-{[(2S,5R)-5- |
| | | | | | Carboxy-3-oxo-2- |
| | | | | | thiomorpholinyl]acetyl}- |
| | | | | | 2,4-didesoxy-1-C- |
| | | | | | {(2S,3R,4S)-3-hydroxy- |
| | | | | | 4- |
| | | | | | [(2R,3S,4E,6E,9S,10S,11R, |
| | | | | | 12E,14Z)-10-hydroxy- |
| | | | | | 3,15-dimethoxy- |
| | | | | | 7,9,11,13-tetramethyl- |
| | | | | | 16- |
| | | | | | oxooxacyclohe;xadeca- |
| | | | | | 4,6,12,14-tetraen-2-yl]-2- |
| | | | | | pentanyl}-5-isopropyl-4- |
| | | | | | methyl-α-D-threo- |
| | | | | | pentopyranose |
| 23 | Temsirolimus | 7.425 | 2.496 | 34.025 | (1R,2R,4S)-4-{(2R)-2- |
| | | | | | [(1R,9S,12S,15R,16E,18R, |
| | | | | | 19R,21R,23S,24E,26E, |
| | | | | | 28E,30S,35R)-1,18- |
| | | | | | Dihydroxy-19,30- |
| | | | | | dimethoxy- |
| | | | | | 15,17,21,23,29,35- |
| | | | | | hexamethyl- |
| | | | | | 2,3,10,14,20-pentaoxo- |
| | | | | | 11,36-dioxa-4- |
| | | | | | azatricyclo[30.3.1.0^{4,9}] |
| | | | | | hexatriaconta- |
| | | | | | 16,24,26,28-tetraen-12- |
| | | | | | yl]propyl}-2- |
| | | | | | methoxycyclohexyl 3- |
| | | | | | hydroxy-2- |
| | | | | | (hydroxymethyl)-2- |
| | | | | | methylpropanoate |
| 24 | LY411575 | 7.426 | 1.716 | 8.675 | N²-[(2S)-2-(3,5- |
| | | | | | Difluorophenyl)-2- |
| | | | | | hydroxyacetyl]-N-[(7S)- |
| | | | | | 5-methyl-6-oxo-6,7- |
| | | | | | dihydro-5H- |
| | | | | | dibenzo[b,d]azepin-7- |
| | | | | | yl]-L-alaninamide |
| 25 | Trametinib | 7.426 | 1.351 | 10.091 | N-(3-{3-Cyclopropyl-5- |
| | | | | | [(2-fluor-4- |
| | | | | | iodphenyl)amino]-6,8- |
| | | | | | dimethyl-2,4,7-trioxo- |
| | | | | | 3,4,6,7- |
| | | | | | tetrahydropyrido[4,3- |
| | | | | | d]pyrimidin-1(2H)- |
| | | | | | yl}phenyl)acetamide |
| 26 | Lubiprostone | 7.426 | 2.218 | 13.564 | 7-[(2R,4aR,5R,7aR)-2- |
| | | | | | (1,1-difluoropentyl)-2- |
| | | | | | hydroxy-6- |
| | | | | | oxooctahydrocyclopenta[b]pyran- |
| | | | | | 5-yl]heptanoic |
| | | | | | acid |
| 27 | Cobimetinib | 7.427 | 1.909 | 13.702 | 3,4-Difiuoro-2-[(2- |
| | | | | | fluoro-4- |
| | | | | | iodopheny)amino]phenyl}{3- |
| | | | | | hydroxy-3-[(2S,)-2- |
| | | | | | piperidinyl]-1- |
| | | | | | azetidinyl}methanone |
| 28 | PLX-4720 | 7.427 | 1.483 | 14.197 | N-{3-[(5-Chloro-1H- |
| | | | | | pyrrolo[2,3-b]pyridin-3- |
| | | | | | yl)carbonyl]-2,4- |
| | | | | | difluorophenyl}-1- |
| | | | | | propanesulfonamide |
| 29 | Vemurafenib | 7.427 | 1.442 | 27.945 | N-{3-[5-(4- |
| | | | | | chlorophenyl)-1H- |
| | | | | | pyrrolo[2,3-b]pyridin-3- |
| | | | | | carbonyl]-2,4- |
| | | | | | difluorophenyl}propane- |
| | | | | | 1-sulfonamide |
| 30 | Verteporfin | 7.428 | 1.868 | 60.28 | 3- |
| | | | | | [(1Z,7Z,12Z,16Z,23S,24R)- |
| | | | | | 22,23- |
| | | | | | Bis(methoxycarbonyl)-5- |
| | | | | | (3-methoxy-3- |
| | | | | | oxopropyl)-4,10,15,24- |
| | | | | | tetramethyl-14-vinyl- |
| | | | | | 25,26,27,28- |
| | | | | | tetraazahexacyclo[16.6.1.1^{3,6}.1^{8,11}.1^{13,16}.0^{19,24}]octacosa- |
| | | | | | 1,3,5,7,9, |
| | | | | | 11(27),12,14,16,18(25), |
| | | | | | 19,21-dodecaen-9- |
| | | | | | yl]propanoic acid |
| 31 | Viomycin | 7.428 | 2.213 | 69.421 | (3S)-3,6-Diamino-N- |
| | | | | | [(3S,6Z,9S,12S,15S)-3- |
| | | | | | [(4R,6S)-2-amino-6- |
| | | | | | hydroxy-1,4,5,6- |
| | | | | | tetrahydro-4- |
| | | | | | pyrimidinyl]-6- |
| | | | | | [carbamoylamino)methylene]- |
| | | | | | 9,12- |
| | | | | | bis(hydroxymethyl)- |
| | | | | | 2,5,8,11,14-pentaoxo- |
| | | | | | 1,4,7,10,13-pentaazacyclohexadecan- |
| | | | | | 15- |
| | | | | | yl]hexanamide |
| 32 | PND-1186 | 7.429 | 1.352 | 18.512 | 2-{[2-{[2-Methoxy-4-(4- |
| | | | | | morpholinyl)phenyl]amino}- |
| | | | | | 5-(trifluoromethyl)- |
| | | | | | 4-pyridinyl]amino}-N- |
| | | | | | methylbenzamide |
| 33 | NVP- | 7.429 | 1.534 | 22.394 | 4-methyl-3-((1-methyl-6- |
| | BHG712 | | | | (pyridin-3-yl)-1H- |
| | | | | | pyrazolo[3,4- |
| | | | | | d]pyrimidin-4-yl)amino)- |
| | | | | | N-(3- |
| | | | | | (trifluoromethyl)phenyl)benzamide |
| 34 | Nilotinib | 7.429 | 1.482 | 25.849 | 4-methyl-N-(3-(4- |
| | | | | | methylimidazol-1-yl)-5- |
| | | | | | (trifluoromethyl)phenyl)- |
| | | | | | 3-((4-pyridin-3- |
| | | | | | ylpyrimidin-2- |
| | | | | | yl)amino)benzamide |
| 35 | OSI-930 | 7.429 | 1.372 | 39.269 | 3-[(4- |
| | | | | | Quinoleinylmethyl)amino]- |
| | | | | | N-[4- |
| | | | | | (trifluoromethoxy)phenyl]- |
| | | | | | 2- |
| | | | | | thiophenecarboxamide |
| 36 | Troleandomycin | 7.43 | 2.1 | 8.405 | (3S,5R,6S,7S,8R,11R,12S, |
| | | | | | 13R,14S,15S)-12-[(4- |
| | | | | | O-acetyl-2,6-dideoxy-3- |
| | | | | | O-methyl-a-L-arabino- |
| | | | | | hexopyranosyl)oxy]-14- |
| | | | | | {[2-O-acetyl-3,4,6- |
| | | | | | trideoxy-3- |
| | | | | | (dimethylamino)-b-D- |
| | | | | | xylo- |
| | | | | | hexopyranosyl]oxy}- |
| | | | | | 5,7,8,11,13,15- |
| | | | | | hexamethyl-4,10-dioxo- |
| | | | | | 1,9- |
| | | | | | dioxaspiro[2.13]hexadec- |
| | | | | | 6-yl acetate |
| 37 | Fluticasone | 7.43 | 2.817 | 8.415 | 6α,11β,16α,17α)-6,9- |
| | | | | | Difluoro-11,17- |
| | | | | | dihydroxy-16-methyl-3- |
| | | | | | oxoandrosta-1,4-diene- |
| | | | | | 17-carbothioate de S- |
| | | | | | (fluoromethyle) |
| 38 | CH4987655 | 7.431 | 1.541 | 6.37 | ,4-Difluor-2-[(2-fluor-4- |
| | | | | | iodphenyl)amino]-N-(2- |
| | | | | | hydroxyethoxy)-5-[(3- |
| | | | | | oxo-1,2-oxazinan-2- |
| | | | | | yl)methyl]benzamid |
| 39 | Paclitaxel | 7.431 | 1.995 | 9.789 | (1S,2S,3R,4S,7R,9S,10S, |
| | | | | | 12R,15S)-4,12- |
| | | | | | bis(acetyloxy)-1,9- |
| | | | | | dihydroxy-15-({(2R,3S)- |
| | | | | | 2-hydroxy-3-phenyl-3- |
| | | | | | [(phenylcarbonyl)amino]propanoyl}oxy)- |
| | | | | | 10,14,17,17-tetramethyl- |
| | | | | | 11-oxo-6- |
| | | | | | oxatetracyclo[11.3.1.0^{3,10}.0^{4,7}]heptadec- |
| | | | | | 13-en-2-yl |
| | | | | | benzoate |
| 40 | Docetaxel | 7.431 | 2.515 | 7.075 | (2α,5β,7β,10β,13α)-4- |
| | | | | | (acetyloxy)-13- |
| | | | | | ({(2R,3S)-3-[(tert- |
| | | | | | butoxycarbonyl)amino]- |
| | | | | | 2-hydroxy-3- |
| | | | | | phenylpropanoyl}oxy)- |
| | | | | | 1,7,10-trihydroxy-9-oxo- |
| | | | | | 5,20-epoxytax-11-en-2- |
| | | | | | yl benzoate |
| 41 | Pluripotin | 7.431 | 1.73 | 22.943 | N-(3-{7-[(1,3-Dimethyl- |
| | | | | | 1H-pyrazol-5-yl)amino]- |
| | | | | | 1-methyl-2-oxo-1,4- |
| | | | | | dihydropyrimido[4,5- |
| | | | | | d]pyrimidin-3(2H)-yl}-4- |
| | | | | | methylphenyl)-3- |
| | | | | | (trifluoromethyl)benzamide |
| 42 | CEP-32496 | 7.431 | 2.035 | 27.97 | 1-{3-[(6,7-Dimethoxy-4- |
| | | | | | quinazolinyl)oxy]phenyl}- |
| | | | | | 3-[5-(1,1,1-trifluoro-2- |
| | | | | | methyl-2-propanyl)-1,2- |
| | | | | | oxazol-3-yl]urea |
| 43 | Sorafenib | 7.432 | 1.575 | 17.437 | 4-[4-({[4-Chloro-3- |
| | | | | | (trifluoromethyl)phenyl]carbamoyl}amino)phenoxy]- |
| | | | | | N-methyl-2- |
| | | | | | pyridinecarboxamide |
| 44 | PF-03814735 | 7.434 | 1.498 | 10.476 | N-{2-[(1R,8S)-4-{[4- |
| | | | | | (Cyclobutylamino)-5- |
| | | | | | (trifluoromethyl)-2- |
| | | | | | pyrimidinyl]amino}-11- |
| | | | | | azatricyclo[6.2.1.0^{2,7}]undeca- |
| | | | | | 2,4,6-trien-11-yl]-2- |
| | | | | | oxoethyl}acetamide |
| 45 | Efavirenz | 7.435 | 1.226 | 19.201 | (4S)-6-Chloro-4- |
| | | | | | (cyclopropylethynyl)-4- |
| | | | | | (trifluoromethyl)-4H-3,1- |
| | | | | | benzoxazin-2-ol |
| 46 | PF-04929113 | 7.439 | 1.963 | 11.155 | trans-4-({2-Carbamoyl- |
| | | | | | 5-[6,6-dimethyl-4-oxo-3- |
| | | | | | (trifluoromethyl)-4,5,6,7- |
| | | | | | tetrahydro-1H-indazol-1- |
| | | | | | yl]phenyl}amino)cyclohexyl |
| | | | | | glycinate |
| 47 | Valrubicin | 7.441 | 1.898 | 9.283 | (2S-cis)-Pentanoic Acid |
| | | | | | 2-(1,2,3,4,6,11- |
| | | | | | hexahydro-2,5,12- |
| | | | | | trihydroxy-7-methoxy- |
| | | | | | 6,11-dioxo-4-((2,3,6- |
| | | | | | trideoxy-3- |
| | | | | | ((trifluoroacetyl)amino)- |
| | | | | | a-L-lyxo- |
| | | | | | hexopyranosyl)oxy)-2- |
| | | | | | naphthacenyl)-2- |
| | | | | | oxoethyl Ester |
| 48 | MK-0752 | 7.474 | 1.922 | 31.253 | 3-(cis-4-((4- |
| | | | | | Chlorophenyl)sulfonyl)- |
| | | | | | 4-(2,5- |
| | | | | | difluorophenyl)cyclohexyl)propanoic |
| | | | | | acid |
| 49 | GSK2636771 | 7.475 | 1.716 | 18.306 | 2-Methyl-1-[2-methyl-3- |
| | | | | | (trifluoromethyl)benzyl]- |
| | | | | | 6-(4-morpholinyl)-1H- |
| | | | | | benzimidazole-4- |
| | | | | | carboxylic acid |
| 50 | Saprisartan | 7.703 | 1.306 | 51.121 | 1-{[3-Bromo-2-(2- |
| | | | | | {[(trifluoromethyl)sulfonyl]amino}phenyl)- |
| | | | | | 1- |
| | | | | | benzofuran-5- |
| | | | | | yl]methyl}-4- |
| | | | | | cyclopropyl-2-ethyl-1H- |
| | | | | | imidazole-5- |
| | | | | | carboxamide |
| 51 | Defactinib | 7.716 | 1.613 | 6.799 | N-Methyl-4-({4-[({3- |
| | | | | | [methyl(methylsulfonyl)amino]- |
| | | | | | 2- |
| | | | | | pyrazinyl}methyl)amino]- |
| | | | | | 5-(trifluoromethyl)-2- |
| | | | | | pyrimidinyl}amino)benzamide |
| 52 | PF-00562271 | 7.716 | 1.572 | 9.678 | N-methyl-N-(3-((2-(2- |
| | | | | | oxo-2,3-dihydro-1H- |
| | | | | | indol-5-ylamino)-5- |
| | | | | | trifluoromethyl- |
| | | | | | pyrimidin-4-ylamino)- |
| | | | | | methyl)-pyridin-2-yl)- |
| | | | | | methanesulfonamide |
| 53 | PF-431396 | 7.716 | 1.547 | 13.857 | N-Methyl-N-{2-[({2-[(2- |
| | | | | | oxo-2,3-dihydro-1H- |
| | | | | | indol-5-yl)amino]-5- |
| | | | | | (trifluoromethyl)-4- |
| | | | | | pyrimidinyl}amino)methyl]phenyl}methanesulfonamide |
| 54 | PF-573228 | 7.716 | 1.585 | 15.743 | 6-(4-(3- |
| | | | | | (methylsulfonyl)benzylamino)- |
| | | | | | 5- |
| | | | | | (trifluoromethyl)pyrimidin- |
| | | | | | 2-ylamino)-3,4- |
| | | | | | dihydroquinolin-2(1H)- |
| | | | | | one |
| 55 | Dabrafenib | 7.716 | 1.816 | 34.224 | N-[3-[5-(2-amino-4- |
| | | | | | pyrimidinyl)-2-(1,1- |
| | | | | | dimethylethyl)-4- |
| | | | | | thiazolyl]-2- |
| | | | | | fluorophenyl]-2,6- |
| | | | | | difluorobenzenesulfonamide |
| 56 | Tipranavir | 7.717 | 1.909 | 54.698 | N-(3-{(1R)-1-[(6R)-4- |
| | | | | | Hydroxy-2-oxo-6-(2- |
| | | | | | phenylethyl)-6-propyl- |
| | | | | | 5,6-dihydro-2H-pyran-3- |
| | | | | | yl]propyl}phenyl)-5- |
| | | | | | (trifluoromethyl)-2- |
| | | | | | pyridinesulfonamide |
| 57 | Navitoclax | 7.719 | 2.072 | 110.149 | 4-[4-[[2-(4- |
| | | | | | Chlorophenyl)-5,5- |
| | | | | | dimethyl-1-cyclohexen- |
| | | | | | 1-yl]methyl]-1- |
| | | | | | piperazinyl]-N-[[4- |
| | | | | | [[(1R)-3-(4- |
| | | | | | morpholinyl)-1- |
| | | | | | [(phenylthio)methyl]propyl]amino]- |
| | | | | | 3- |
| | | | | | [(trifluoromethyl)sulfonyl]phenyl]sulfonyl]benzamide |
| 58 | Celecoxib | 7.726 | 1.381 | 20.716 | 4-[5-(4-Methylphenyl)- |
| | | | | | 3-(trifluoromethyl)-1H- |
| | | | | | pyrazol-1- |
| | | | | | yl]benzenesulfonamide |
| 59 | Flutamide | 7.815 | 1.732 | 8.506 | 2-Methyl-N-[4-nitro-3- |
| | | | | | (trifluormethyl)phenyl]propanamid |
| 60 | Bicalutamide | 7.999 | 1.991 | 8.566 | N-[4-Cyan-3- |
| | | | | | (trifluormethyl)phenyl]- |
| | | | | | 3-[(4- |
| | | | | | fluorphenyl)sulfonyl]-2- |
| | | | | | hydroxy-2- |
| | | | | | methylpropanamid |
| 61 | RO4929097 | 7.999 | 1.838 | 10.833 | 2,2-Dimethyl-N-[(7S)-6- |
| | | | | | oxo-6,7-dihydro-5H- |
| | | | | | dibenzo[b,d]azepin-7- |
| | | | | | yl]-N′-(2,2,3,3,3- |
| | | | | | pentafluoropropyl)malonamide |
| 62 | Avagacestat | 7.999 | 1.613 | 15.962 | (R)-2-(4-chloro-N-(2- |
| | | | | | fluoro-4-(1,2,4- |
| | | | | | oxadiazol-3- |
| | | | | | yl)benzyl)phenylsulfonamido)- |
| | | | | | 5,5,5- |
| | | | | | trifluoropentanamide |
| 63 | Mefloquine | 7.999 | 1.912 | 18.51 | (S)-[2,8- |
| | | | | | Bis(trifluormethyl)-4- |
| | | | | | chinolinyl][(2R)-2- |
| | | | | | piperidinyl]methanol |
| 64 | Enzalutamide | 7.999 | 1.998 | 18.789 | 4-(3-(4-Cyano-3- |
| | | | | | (trifluoromethyl)phenyl)- |
| | | | | | 5,5-dimethyl-4-oxo-2- |
| | | | | | thioxoimidazolidin-1-yl)- |
| | | | | | 2-fluoro-N- |
| | | | | | methylbenzamide |
| 65 | Regorafenib | 7.999 | 1.65 | 19.196 | 4-[4-({[4-Chloro-3- |
| | | | | | (trifluoromethyl)phenyl]carbamoyl}amino)- |
| | | | | | 3- |
| | | | | | fluorophenoxy]-N- |
| | | | | | methyl-2- |
| | | | | | pyridinecarboxamide |
| 66 | Aprepitant | 7.999 | 1.773 | 23.513 | 5-{[(2R,3S)-2-{(1R)-1- |
| | | | | | [3,5- |
| | | | | | bis(trifluoromethyl)phenyl]ethoxy}- |
| | | | | | 3-(4- |
| | | | | | fluorophenyl)morpholin- |
| | | | | | 4-yl]methyl}-1,2- |
| | | | | | dihydro-3H-1,2,4-triazol- |
| | | | | | 3-one |
| 67 | Dutasteride | 7.999 | 2.629 | 32.536 | (4aR,4bS,6aS,7S,9aS,9bS, |
| | | | | | 11aR)-N-[2,5- |
| | | | | | Bis(trifluoromethyl)phenyl]- |
| | | | | | 4a,6a-dimethyl-2- |
| | | | | | oxo- |
| | | | | | 2,4a,4b,5,6,6a,7,8,9,9a,9b, |
| | | | | | 10,11,11a- |
| | | | | | tetradecahydro-1H- |
| | | | | | indeno[5,4-f]quinoline-7- |
| | | | | | carboxamide |
| 68 | TAK-632 | 7.999 | 1.635 | 32.949 | N-{7-Cyano-6-[4-fluoro- |
| | | | | | 3-({[3- |
| | | | | | (trifluoromethyl)phenyl]acetyl}amino)phenoxy]- |
| | | | | | 1,3-benzothiazol-2- |
| | | | | | yl}cyclopropanecarboxamide |
| 69 | antrafenine | 7.999 | 1.801 | 50.049 | 2-[4-(a,a,a-trifluoro-m- |
| | | | | | tolyl)-1- |
| | | | | | piperazinyl]ethyl-n-(7- |
| | | | | | trifluoromethyl-4- |
| | | | | | quinolyl)anthranilate |
| 70 | Lomitapide | 7.999 | 1.797 | 60.697 | N-(2,2,2-Trifluoroethyl)- |
| | | | | | 9-{4-[4-({[4′- |
| | | | | | (trifluoromethyl)-2- |
| | | | | | biphenylyl]carbonyl}amino)- |
| | | | | | 1-piperidinyl]butyl}- |
| | | | | | 9H-fluorene-9- |
| | | | | | carboxamide |
| 71 | Fulvastrant | 7.999 | 2.464 | 70.768 | (7α,17β)-7-{9- |
| | | | | | [(4,4,5,5,5- |
| | | | | | Pentafluoropentyl)sulfinyl]nonyl}estra- |
| | | | | | 1(10),2,4- |
| | | | | | triene-3,17-diol |

Table 4 shows molecular descriptors of 37 experimentally validated drugs, descriptors correlating highly (coeff.>0.8) to experimental data for nanoparticle formation (DLS/visual precipitation) and the corresponding score for each drug. Dashed box denotes compound which formed nanoparticles with indocyanine. All other drugs did not form stable suspensions.

The molecular descriptors included in Table 4 are the following:

ZM1Kup: First Zagreb index by Kupchik vertex degrees;

Psi_i_s: Intrinsic state pseudoconnectivity index—type S;

HyWi_B(s): Hyper-Wiener-like index (log function) from Burden matrix weighted by I-State;

SpPos_B(s): Spectral positive sum from Burden matrix weighted by I-State;

SpAD_B(s): Spectral absolute deviation from Burden matrix weighted by I-State;

SM5_B(s): Spectral moment of order 5 from Burden matrix weighted by I-State;

ATS2s: Broto-Moreau autocorrelation of lag 2 (log function) weighted by I-state;

H4s: H autocorrelation of lag 4/weighted by I-state; and

RTs: R total index/weighted by I-state.

However, additional molecular descriptors can be used in alternative embodiments.

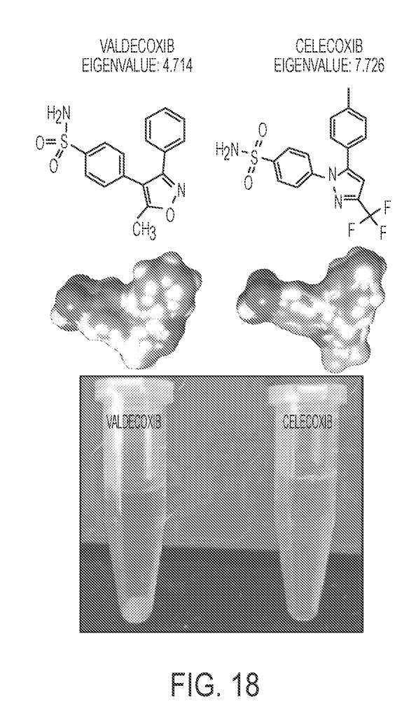

The validation set revealed a common property among most drugs exhibiting high eigenvalues—the presence of at least one fluorine atom. Of the 63 molecules predicted, 60% had one or more fluorine atoms. To confirm the importance of fluorine to the calculated descriptor values, very similar drugs were compared with and without fluorine. The structural screen revealed two similar molecules, celecoxib, with 3 fluorine atoms and a calculated eigenvalue of 7.7, and valdecoxib, with zero fluorines and a calculated eigenvalue 4.7. The drugs behaved experimentally as predicted, where the high-eigenvalue molecule formed nanoparticles but the low-eigenvalue molecule did not (FIG. 18). Without wishing to be bound to any theory, the presence of fluorine in medicinal compounds tends to increase stability, hydrophobicity, and electronegativity of drug molecules.

QSAP was employed to additionally predict nanoparticle size using the molecular structure information of drugs. A training set of 8 drug molecules was generated by measuring the sizes of nanoparticles formed by nano-precipitation with indocyanine. It was found that a molecular descriptor, GETAWAY R4e, correlated significantly with nanoparticle size data (coeff=0.83). This descriptor is based on the leverage matrix from the spatial coordinates of a molecule using molecular weightings derived from atomic mass, polarizability, van der Waals volume, and electronegativity. A validation set was then generated by calculating this descriptor for an additional 10 nanoparticle-forming drugs and measured the INP sizes experimentally. The resulting nanoparticle sizes were successful predicted by the GETAWAY R4e descriptor, within an error of ±15 nm (FIGS. 10D-10E).

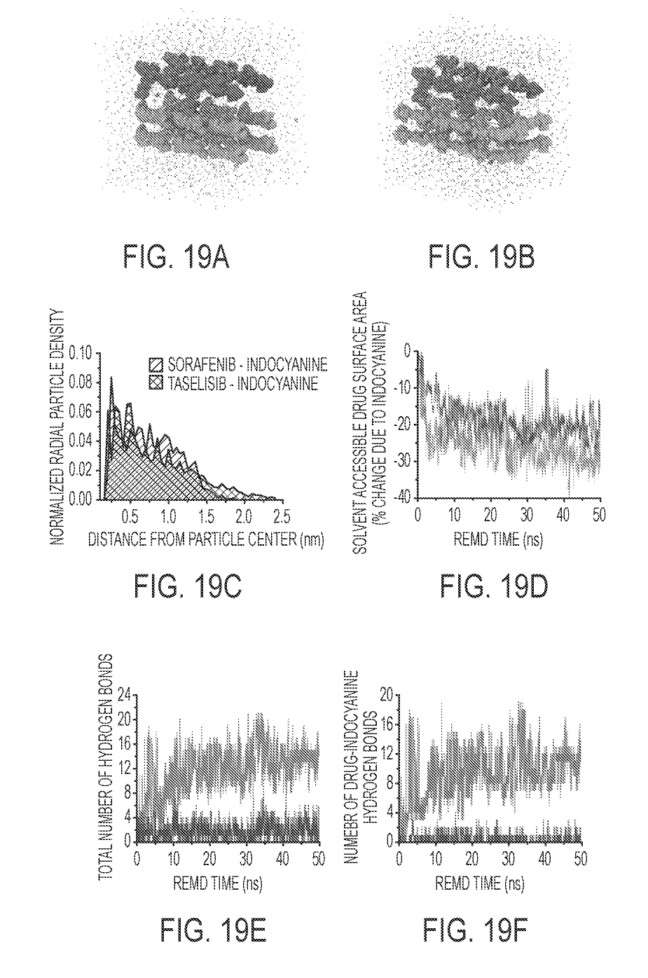

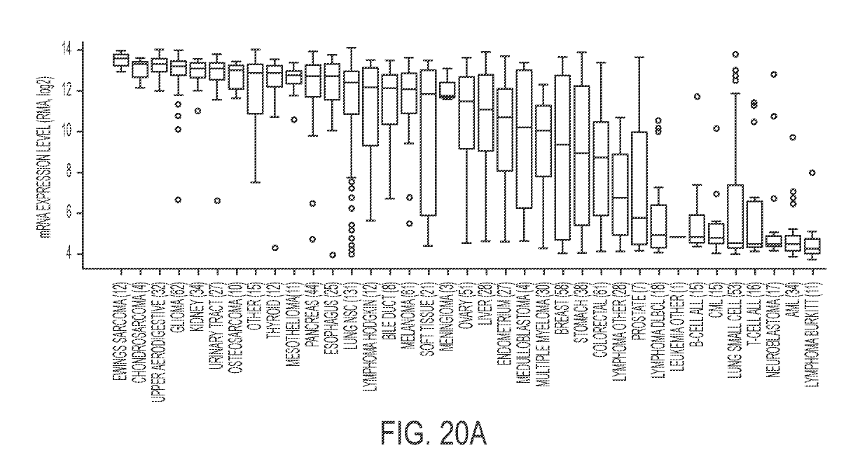

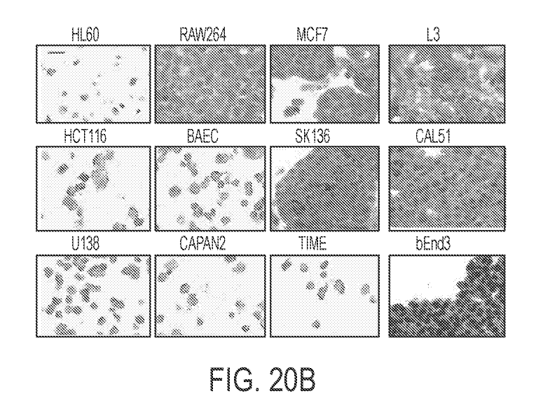

To better understand the self-assembly process, all-atom replica exchange molecular dynamics (REND) simulations were conducted. The assembly of the indocyanine with two drug molecules, sorafenib and taselisib, as representative of high and low eigenvalues, respectively, were investigated. Four indocyanine molecules and twelve drug molecules (either sorafenib or taselisib) were included in a box with explicit water and run with 32 different temperature replicas for 50 ns (FIGS. 20A-20B). The simulations resulted in the formation of indocyanine-drug assemblies with clear morphological differences (FIG. 10F). The sorafenib-indocyanine simulation formed a significantly more compact assembly than the taselisib-indocyanine simulation. In the taselisib simulation, the resulting complex remained relatively loosely assembled, as evident from the relative radial particle density (FIG. 19C) and solvent accessibility to the drug molecules (FIG. 10G). Without wishing to be bound to any theory, these differences may be the in the number of hydrogen bonds formed in the two simulations. The sorafenib-indocyanine complex formed over four-fold more hydrogen bonds compared to the taselisib-indocyanine complex (p<0.001, FIG. 10H), mainly between the drug molecules themselves (FIG. 19D).

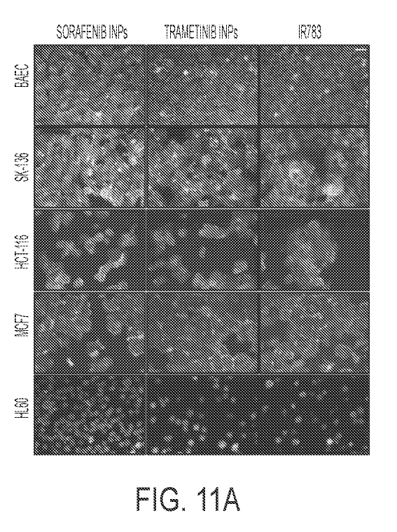

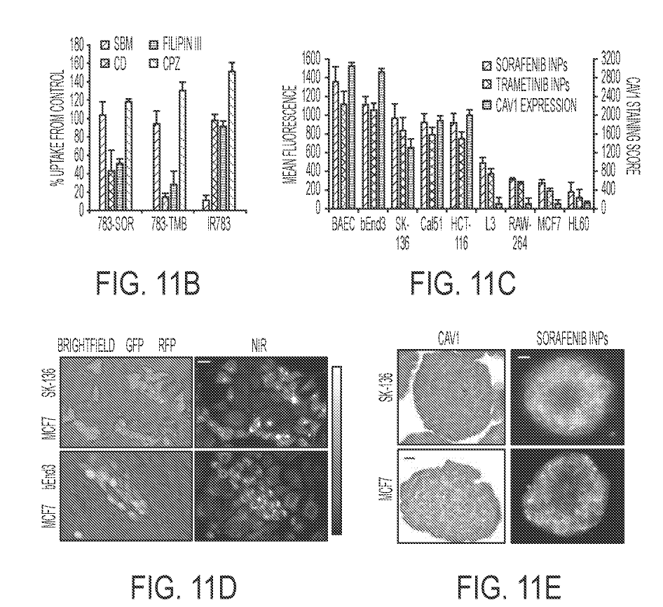

Two INPs encapsulating kinase inhibitors were synthesized for targeting and anti-tumor efficacy studies. Nanoparticles encapsulating sorafenib, a multikinase inhibitor, and trametinib, a MEK inhibitor, were 80 nm and 55 nm in diameter, and exhibited drug loadings of 86% and 83%, respectively. The internalization of these nanoparticles was studied in multiple cell lines chosen to represent a range of cell types: endothelial cells, epithelial cancers, leukemia, lymphomas, and fibroblasts. Differential uptake was observed across cell types, with a significant preference for endothelial cells and colon and liver cancer cells (FIG. 11A).

The mechanism of nanoparticle uptake using various inhibitors of endocytic pathways. Inhibitors of caveolin-mediated endocytosis, but not clathrin-mediated endocytosis, significantly attenuated nanoparticle uptake (FIG. 11B). Nanoparticles coated with highly sulfated and aromatic polystyrene sulfonate exhibit differential uptake in cells via caveolae. Without wishing to be bound to any theory, it was hypothesized that the INPs, incorporating a high loading of hydrophobic drug and sparsely coated by sulfated indocyanines, may elicit similar caveolae-targeting effects.

To further investigate the caveolae-targeting hypothesis, the human protein atlas and Broad Institute cancer cell line encyclopedia (CCLE) databases were tested for expression levels of CAV1, the main protein scaffold of caveolae (FIG. 20A). It was found that a significant correlation (R²=0.86) between CAV1 expression, assessed by immunohistochemistry (FIG. 20B), and the nanoparticle fluorescence signal across cell lines (FIG. 11C). This preference was also demonstrated in a co-culture of breast and liver cell lines, where the CAV1-positive cells exhibited greater nanoparticle uptake, in bEnd.3 and SK-136 cells lines over MCF-7 (FIG. 11D).

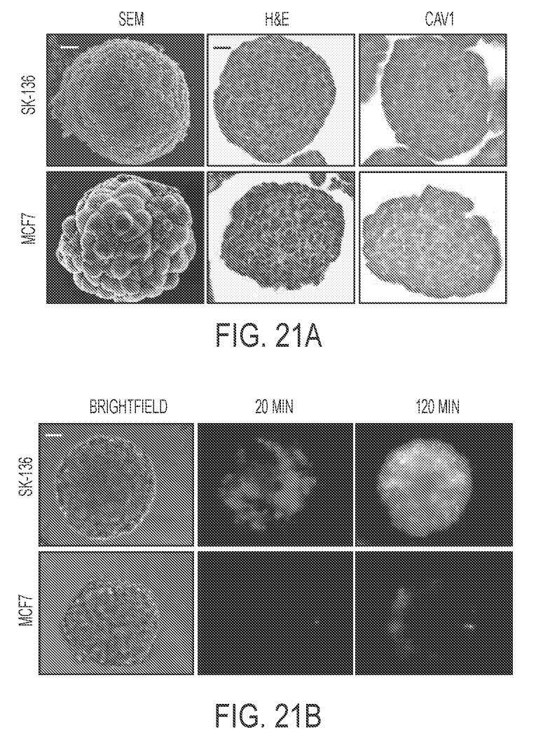

The ability of the nanoparticles was assessed to target three-dimensional tumor spheres in vitro. MCF-7 and SK-136 cells formed tight multi-cellular tumor spheroids with diameters ranging from 200-300 μm (FIG. 11E). Fluorescent imaging of the tumor spheres after 40 min of incubation with 20 μg/ml of sorafenib INPs or trametinib INPs revealed a similar pattern to the 2-D cell culture experiments, preferentially targeting the CAV1-expressing SK-136 tumor spheres (FIG. 11E). Furthermore, the nanoparticles exhibited significantly more penetration in SK-136 tumor spheres compared to the MCF-7 spheres over a period of 120 min (FIGS. 21A and 21B). It was noted that this penetration did not likely occur due to interstitial spacing or permeability of the spheres, as the MCF-7 spheres were previously found to exhibit more interstitial spacing and be more permeable than the SK-136 spheres to nanomaterials.

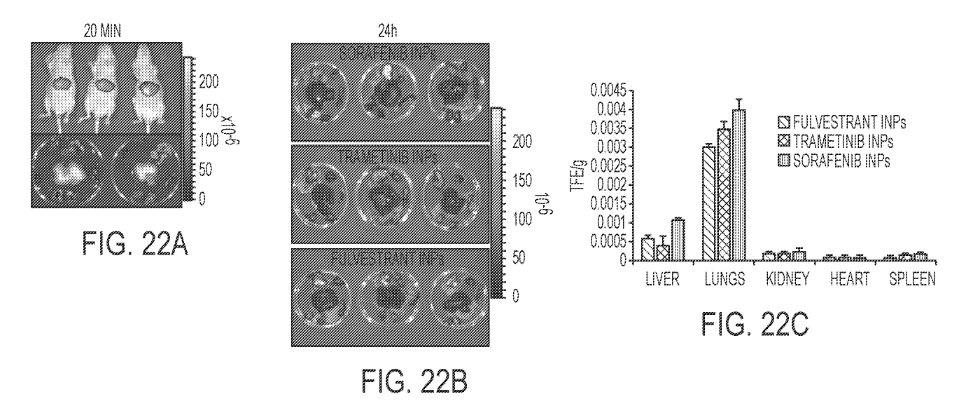

The biodistribution of the indocyanine nanoparticles were measured in healthy mice. After intravenous administration by tail vein injection, the nanoparticles appeared in the liver first—within 20 min. The near-infrared signal from the indocyanine in the lungs increased from 0-24 hours (FIGS. 22A-22C). The significant lung accumulation is due to the large number of endothelial cells and caveolae present in these organs. Notably, low signal in the liver and the absence of signal in the spleen was observed, potentially indicating low uptake by macrophages.

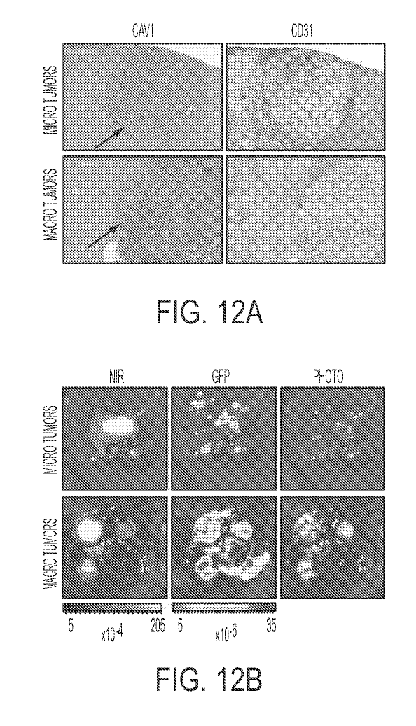

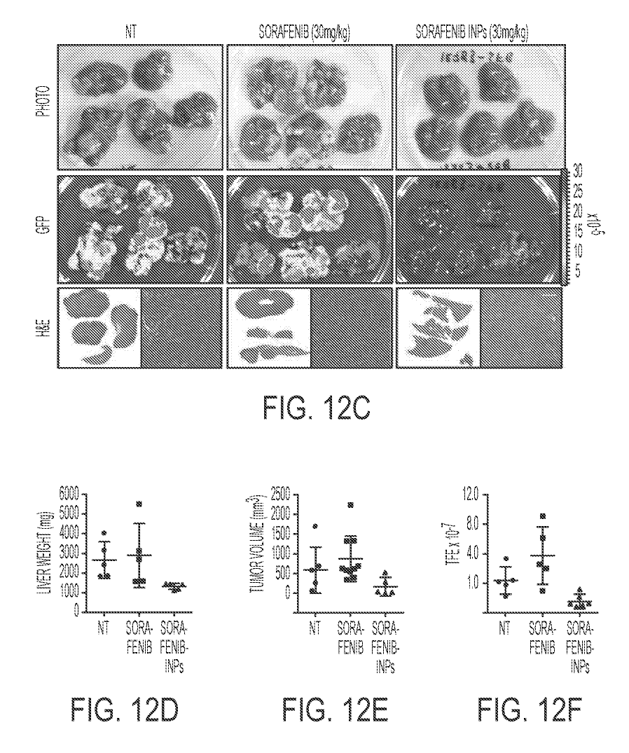

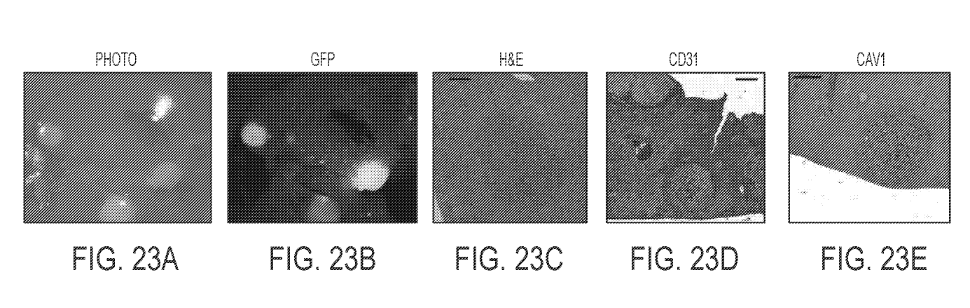

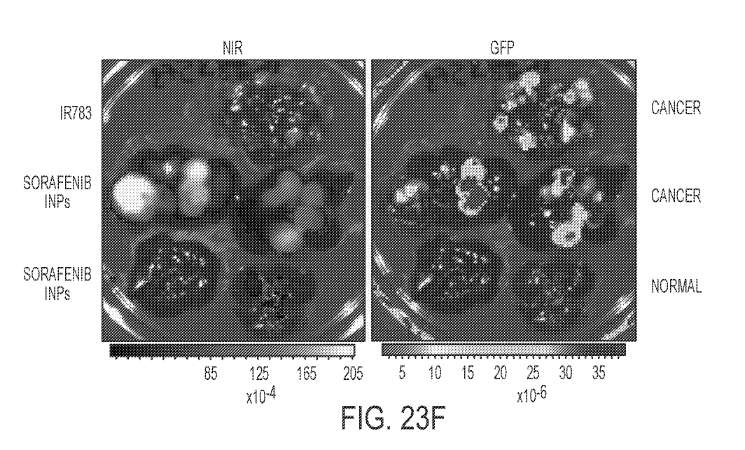

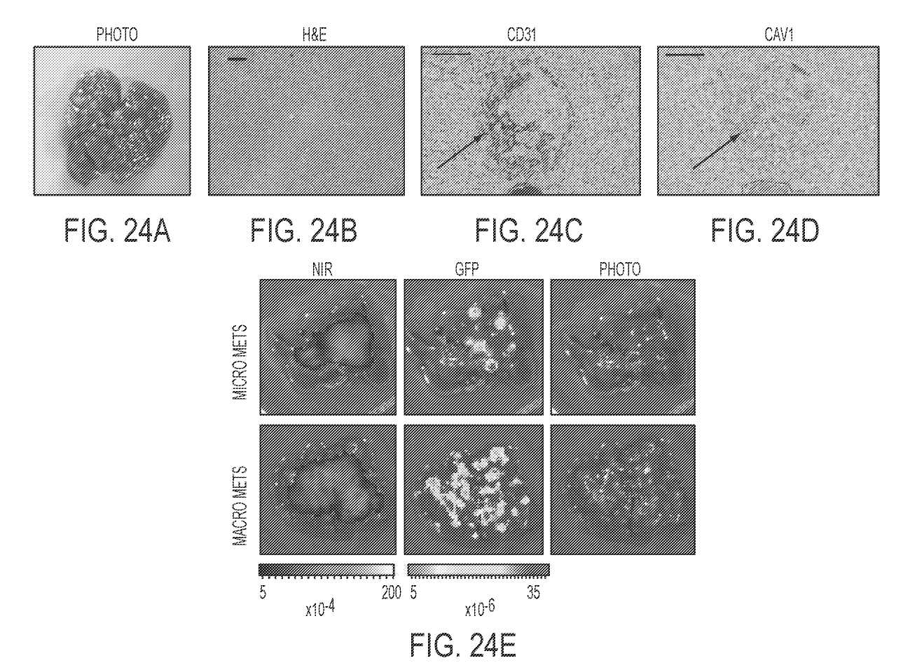

Next, the biodistribution of INPs in a MYC-driven murine hepatic tumor model was evaluated. To generate autochthonous liver tumors, Sleeping Beauty transposon vectors encoding c-Myc and mutant β-catenin (coupled to GFP) were hydrodynamically delivered into immunocompetent FVB mice along with a vector encoding Sleeping Beauty transposase as described herein. At three weeks after inoculation, tumor nodules could be detected in the liver (FIGS. 23A-23C), and antibody stains of CAV1 revealed its presence in virtually all tumor vessels as well as larger vessels of normal liver tissue (FIG. 12A, FIGS. 21D-23E). Fluorescence imaging of livers harvested 24 h after i.v. injection of sorafenib-encapsulated indocyanine nanoparticles revealed localization of the nanoparticles specifically within tumor tissue, indicated by co-localization of sorafenib INPs and GFP-positive areas (FIG. 12B). However, no significant accumulation of Sorafenib INPs was observed in normal liver, and free indocyanine did not accumulate in tumors (FIG. 23F). Moreover, without wishing to be bound to any theory, sorafenib INPs exhibited little accumulation in liver metastases of uveal melanoma tumors, which lack CAV1 (FIGS. 24A-24E), suggesting that CAV1 expression mediates specific uptake of sorafenib INPs in vivo.

To test the therapeutic potential of the INPs in vivo, the anti-tumor efficacy of equivalent drug doses in the liver cancer mouse model described above were either delivered intravenously via INPs or the orally administered free drug. Sorafenib or sorafenib INPs were injected weekly over the course of three weeks. Notably, whereas mice treated with free sorafenib exhibited multiple liver tumors at the experimental endpoint of 60 days (FIG. 12C), the livers of mice treated with nanoparticles containing the same sorafenib concentration showed virtually no residual tumor tissue, evident by visual inspection, GFP imaging, and histology (FIG. 12C). Furthermore, quantitative measures of liver weight, tumor volume, and GFP emission confirmed virtually complete tumor eradication in INP-treated livers (FIGS. 12D-12F).

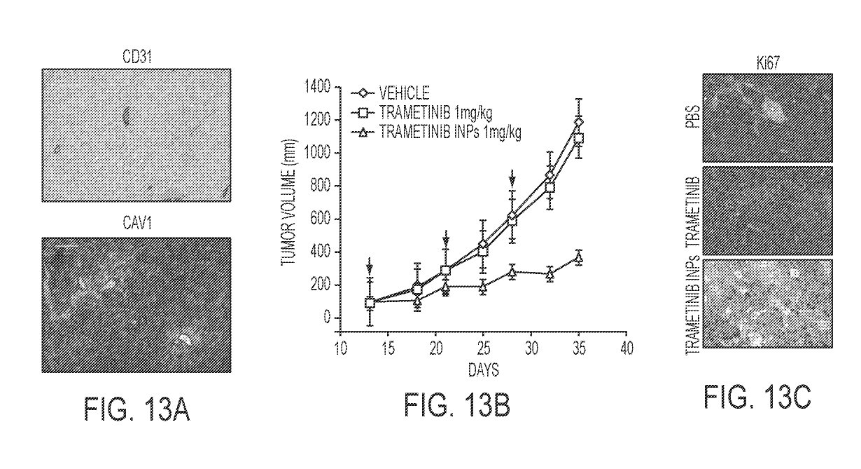

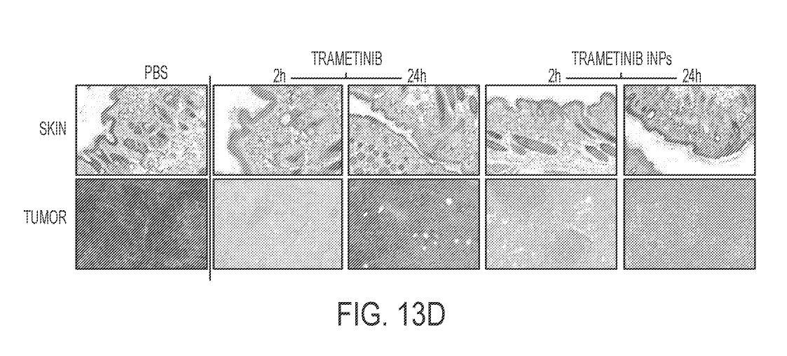

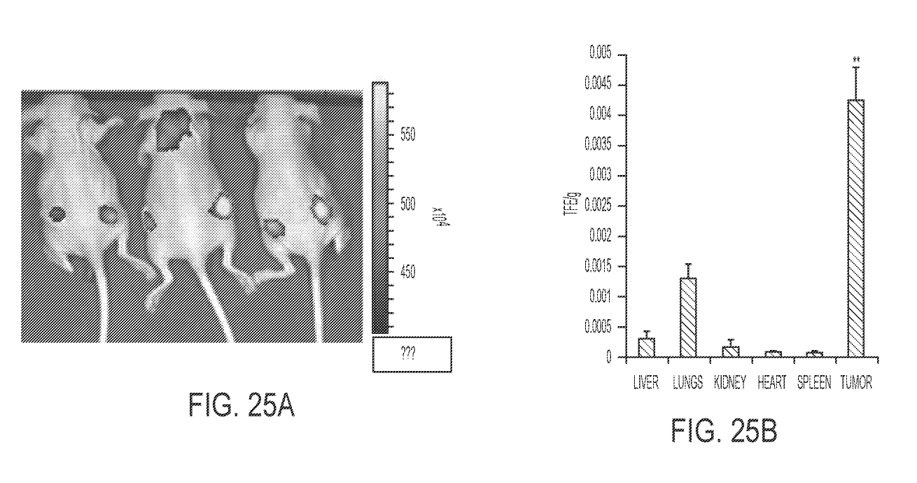

The biodistribution and anti-tumor efficacy of INPs encapsulating the MEK inhibitor trametinib in a CAV1-expressing colon cancer model which is sensitive to MEK inhibition was tested. The subcutaneous HCT116 human colorectal carcinoma model expresses CAV1 in cancer cells and tumor-associated endothelium (FIG. 13A). The nanoparticle biodistribution was assessed in subcutaneous HCT116 xenografts and substantial nanoparticle accumulation in tumors were observed (FIGS. 25A-25B). Nanoparticle fluorescence in the tumor tissue was five-fold higher than in the lungs after 24 h. Comparison of intravenous administration of trametinib INPs to oral administration of free trametinib was investigated. As in the previous experiment, a weekly dose of trametinib did not affect tumor progression, but the nanoparticles, encapsulating an equivalent dose of drug, elicited significant tumor inhibition (FIG. 13B). Tumor proliferation was evaluated using the Ki67 proliferation marker, and a significant reduction in proliferating cells in tumor tissue of mice administered trametinib INPs compared to mice administered trametinib alone was found (FIG. 13C).

As one of the most limiting side effects of systemic MEK inhibition in humans is severe skin rash, the effects of the differential distribution of INPs on MEK inhibition in the tumor and the skin was evaluated. The downstream phosphorylation status of ERK as a marker for drug activity at several time points was used. The systemic distribution of trametinib caused a pronounced inhibition in ERK phosphorylation in the skin and tumor at 2 h, but pERK returned in both after 24 h. In contrast, the nanoparticles elicited prolonged pERK inhibition in the tumor, after 24 h, but minimal inhibition in the skin was apparent at either time point (FIG. 13D)

The results described herein showed that the self-assembly of a drug carrier nanoparticle composed of small molecules can be predicted and understood with an unprecedented degree of certainty via computational methods, such as using molecular structure information as the original inputs. Without wishing to be bound to any theory, this is the first time a computational process predicted the self-assembly of small molecule drugs into a nanoparticle. Using the QSAP process, 63 approved and investigational drugs were predicted to assemble to indocyanine nanoparticles. The nanoparticles exhibited extremely high drug loadings of up to 90%. Representative nanoparticles, incorporating two kinase inhibitors, sorafenib and trametinib, selectively targeted CAV1-expressing human colon cancer and autochthonous liver cancer models to yield therapeutic effects while avoiding pERK inhibition in healthy skin. The possibility to predict the ability to synthesize a targeted nanoparticle a priori using molecular structure information of drug compounds presents a significant advancement in the field of drug delivery by allowing computational methods to facilitate a process that is normally conducted by trial-and-error bench chemistry.

### Quantitative Self-Assembly Prediction (QSAP)

Molecular structure files, obtained from ChemSpider.com, were selected for solubilities of under 0.1 mg/ml in water and energy minimized using ChemBio3D Ultra 14 Suite. A library of 4886 molecular descriptors were calculated for each molecular structure using DRAGON6 software (talette). The descriptors were correlated to the binary experimental observations of nanoparticle formation, confirmed from DLS (entered as a rank of 5 in the vector) or precipitation (denoted as a rank of 0) from visual inspection.

### Molecular Dynamics Simulations of INP Self-Assembly

Four indocyanine molecules and twelve drug molecules sorafenib or taselisib were placed in a 5 nm×5 nm×5 nm water-box with periodic boundary conditions containing approximately 3,700 TIP3P model water molecules and sodium counter-ions to balance the negative charges of the indocyanine. The total system was comprised of ˜12,000 atoms. To run the REMD simulations, the Gromacs 4.6.7 simulation package was used with the Charmm36 force field. Long-range electrostatics were calculated using the particle mesh Ewald method with a 0.9 nm real space cutoff. For van der Waals interactions, a cutoff value of 1.2 nm was used. Simulation parameters for the indocyanine and drug particles were obtained from SwissParam (Swiss Institute of Bioinformatics). The indocyanine-drug configurations were energy minimized and subjected to 100 ps NVT equilibration at 300 K. Thirty-two replicas of the configuration were created with temperatures ranging from 300 K to 563 K. Temperature intervals increased with absolute temperature to maintain uniform exchange probability around 10% acceptance. The 32 replicas were run in parallel for 50 ns of NVT production. Exchange between adjacent temperatures replicas was attempted every 2 ps. The time step of the simulation was 2 fs. The trajectories were saved every 10 ps, yielding a total of 5,000 snapshots for production analysis. Structures were visualized in VIVID.

### MYC/β-Catenin Driven Liver Tumor Studies

Hydrodynamic transfection was performed. Specifically, 10 μg pT3-EF1a-c-myc, 10 μg pT3-EF1a-β-CateninT41A-IRES-GFP and CMV-SB13 Sleeping Beauty transposase (1:5 ratio) were mixed in sterile saline solution. A total volume of plasmid-saline solution mix corresponding to 10% of the body weight was injected into the lateral tail vein of 6- to 8-week old female FVB/N mice (Jackson Laboratory, Maine, USA) within 5-7 seconds. Mice were administered either 30 mg/kg sorafenib orally, or 30 mg/kg sorafenib in sorafenib INP form via tail vein injection. For targeting and biodistribution experiments, mice were injected with sorafenib INPs or indocyanine 3 weeks and 6 weeks after transfection. Livers were harvested 24 hours after injection. For efficacy studies, treatments were administered weekly for three weeks. Livers were harvested at day 59. Tumors were evaluated using fluorescence imaging (IVIS imaging system, Xenogen Corp., Hopkinton, Mass.) and immunohistochemistry (H&E). Tumor volume was measured using a caliper. Mice were maintained and treated in accordance with the institutional guidelines at Memorial Sloan Kettering Cancer Center.

### Drug Release Measurements

Nanoparticles were incubated in PBS at pH 5.5 or 7.4 at 37° C. with a concentration equivalent to 1 μM of drug. The amount of released drug was determined by extracting into ethanol and measuring absorbance at 260 nm using a UV-VIS-NIR spectrophotometer (Jasco 670) or plate reader (Tecan infinite M1000). All experiments were carried out in duplicate.

### Nanoparticle Uptake in Cell Lines

Cell lines bEnd.3, BAEC, SK136, L3, MCF7, HL60 were used. The cells were plated in a 24 well plate (50,000 cells in 1 ml) and incubated with 20 m/ml of nanoparticles for 45 min and another 15 min with CellMask Green (Life Technologies) for membrane staining and Hoescht 33342 (Life technologies) for nuclear staining. The cells were rinsed twice with PBS. Images were acquired with an inverted Olympus IX51 fluorescent microscope equipped with XM10IR Olympus camera and Excite Xenon lamp. Similar exposure times and excitation intensities were applied throughout all experiments. Filter sets: cell membrane: ex 488 nm, em 525 nm, nucleus: ex 350 nm, em 460 nm, IR783 dye in particles: ex 780 nm, em 820 nm. Images were processed with ImageJ software.

### Development of Tumor Spheroids

To generate multi-cellular tumor spheroids, we developed a cell line, SK-136, derived from the autochthonous liver cancer model. The cells were generated and harvested from c-MYC/β-catenin amplified hepatoblastoma cells from FVB mice. The harvested cells were plated on ultra-low attachment 96-well plates (Corning) and incubated for 3 days. The wells were examined with an inverted light microscope to confirm the formation of multicellular tumor spheroids. The wells containing tumor spheres were centrifuged, trypsinized, and seeded in 75 cm cell culture treated flasks with DMEM. This process was repeated 3 times to generate a sub-clone of spheroid-forming cells. To identify CAV1 expression in 7 day-old tumor spheroids, spheriods were collected at the bottom of an Eppendorf tube, suspended in PFA, and embedded in paraffin. 10 μm slices were stained with anti-caveolin-1 antibody (Cell Signaling, cat#3267, 1 ug/ml) as well as H&E. To characterize the surface of the tumor spheroids, SK-136 cells were grown in ultra-low attachment flasks (Corning) for 5 days. Once the spheres were formed, the media containing tumor spheres was removed and placed in 1 ml Eppendorf tube. The spheres were allowed to settle by gravity for 2 min and the media was replaced with fresh media. The spheroids were placed on poly-1-lysine-coated plastic coverslips (Thermonex). The spheroids were then fixed in 2.5% paraformaldehyde in 0.075M cacodylate buffer for one hour, rinsed in cacodylate buffer, and dehydrated in a graded series of alcohols: 50%, 75%, 95% and 100%. The samples were then dried in a JCP-1 Critical Point Dryer (Denton). The coverslips were attached to SEM stubs and sputter-coated with gold/palladium using a Desk IV sputter system (Denton Vacuum). The images were obtained in a Scanning Field Emission Supra 25 scanning electron microscope (Zeiss).

### Penetration of Nanoparticles in Tumor Spheroids

104 SK-136 cells were seeded in 25 cm² ultra-low attachment flasks (Corning) and grown for 7 days in DMEM with media replacement every 3 days. When spheres reached a diameter of approximately 250 μm, 0.2 ml of growth suspension was plated in normal adhesion 96-well plates, yielding 3-5 spheres per well. After 30 min, spheres adhered to the bottom of the wells. Nanoparticles were added at a concentration of 50 μg/ml and incubated for 20-40 min. The wells were washed 3 times with HBSS buffer and imaged with an inverted Olympus IX51 fluorescence microscope equipped with a XM10 Olympus CCD camera. The fluorescence intensity was analyzed using ImageJ software.

### Colon Cancer Xenograft Studies

Six-week-old female athymic NU/NU nude mice purchased from Charles River Laboratories were injected with 5×105 of HCT116 human colorectal carcinoma cells subcutaneously in 100 ml culture media/Matrigel (BD Biosciences) at a 1:5 ratio. Animals were randomized at a tumor volume of 70 to 120 mm3 into four to six groups, with n=8-10 tumors per group. Animals were treated p.o. with trametinib (1 mg/kg) or i.v. with trametinib INPs (1 mg/kg) once a week. Tumor size was measured with a digital caliper, and tumor volumes were calculated using the formula: (length×width2)×(π/6). Animals were euthanized using CO₂ inhalation. Mice were housed in air-filtered laminar flow cabinets with a 12-hr light/dark cycle and food and water ad libitum. Mice were maintained and treated in accordance with the institutional guidelines of Memorial Sloan Kettering Cancer Center. Animal experiments were approved by Memorial Sloan Kettering Cancer.

### Liver Metastasis Model of Uveal Melanoma in NOD SCID GAMMA (NSG Mice)

Human liver metastatic-enriched uveal melanoma cells expressing GFP-luciferase (L3) were supplied by V.K.R. 5×105 cells were injected via the retro-orbital sinus on NSG mice (JAX laboratories). The appearance of liver metastases by bioluminescence was observed within 14 days after inoculation. Nanoparticles were injected 24 h before imaging with (IVIS imaging system, Xenogen Corp., Hopkinton, Mass.).

### Immunohistochemistry

For xenograft samples, dissected tissues were fixed immediately after removal in a 10% buffered formalin solution for a maximum of 24 h at room temperature before being dehydrated and paraffin embedded under vacuum. The tissue sections were deparaffinized with EZPrep buffer (Ventana Medical Systems). Antigen retrieval was performed with CC1 buffer (Ventana Medical Systems), and sections were blocked for 30 minutes with Background Buster solution (Innovex).

The immunohistochemical detection was performed at Molecular Cytology Core Facility of Memorial Sloan Kettering Cancer Center using Discovery XT processor (Ventana Medical Systems). All the tissues were harvested from mice and fixed in 4% PFA overnight. Fixed tissues were dehydrated and embedded in paraffin before 5 μm sections were put on slides. The tissue sections were deparaffinized with EZPrep buffer (Ventana Medical Systems), antigen retrieval was performed with CC1 buffer (Ventana Medical Systems) and sections were blocked for 30 minutes with Background Buster solution (Innovex) or 10% normal rabbit serum in PBS (for CAV1 staining). CAV1 sections were incubated with antibodies against caveolin-1 (Cell Signaling, cat#3267, 1 ug/ml) for 5 h, followed by 60 minutes of incubation with biotinylated rabbit anti-goat IgG (Vector, cat #BA-5000) at 1:200 dilution. pMAPK sections were blocked with avidin/biotin block for 12 minutes, followed by incubation with pMAPK antibodies (Cell Signaling, cat#4370, 1 ug/ml) for 5 h, followed by 60 minutes incubation with biotinylated goat anti-rabbit IgG (Vector labs, cat#PK6101) at 1:200 dilution. Ki67 sections were incubated with Ki67 antibodies (Vector, cat# VP-K451, 0.4 ug/ml) for 5 h, followed by 60 minutes incubation with biotinylated goat anti-rabbit IgG (Vector labs, cat#PK6101) at 1:200 dilution. CD31 sections were incubated with CD31 antibodies (Dianova, cat# DIA-310, 1 ug/ml) for 5 h, followed by 60 minutes incubation with biotinylated rabbit anti-rat IgG (Vector labs, cat#PK-4004) at 1:200 dilution. Detection was performed with a DAB detection kit (Ventana Medical Systems) according to manufacturer instructions, followed by counterstaining with hematoxylin (Ventana Medical Systems) and coverslipped with Permount (Fisher Scientific).

### Molecular Dynamics Modeling of Self-Assembly

Clustering of the REMD trajectory was used to determine the most populous conformation in the simulation. Accounting for an initial equilibration period, the final 25 ns of the 300 K replica trajectory (temperature at which the experiment was performed) was used for all analysis. A native Gromacs clustering algorithm (g cluster) was used with a root mean square deviation (RMSD) cutoff of 1.2 nm based upon the spatial positions of the drug atoms. The top cluster from the 5,000 available snapshots represented 9.6% and 0.8% of the trajectory for the Sorafenib and Taselisib simulations, respectively (FIGS. 10A-10H). Without wishing to be bound to any theory, the significantly lower percentage of trajectory in the top cluster in the Taselisib simulation suggests an intrinsically more random preferred conformation for this drug-dye combination. Normalized radial particle density histograms were constructed from the top cluster configurations (FIGS. 19A-19F).

The solvent accessibilities to the surfaces of the drugs were analyzed to determine accessibility in the complexes. Water and ion accessibilities were analyzed using the Gromacs function ‘g_sas’. In order to compare across the two simulations with differing drug surface areas, the amount of exposed drug to the solvent was quantified with the dye present, and additionally with the dye removed from the trajectory. The percentage change in solvent accessible drug surface area was quantified, revealing that the dye shields the Sorafenib significantly more than Taselisib, 27.9±3.1% vs. 20.3±3.7% (FIG. 19D).

Hydrogen bonding analysis was performed using the Gromacs function ‘g_hbond’. The total number of hydrogen bonds between solute molecules in the system and between dye and drug molecules were calculated (FIGS. 19E-19F). The dye was not able to hydrogen bond to itself, and thus the total number of bonds comprised drug-drug and dye-drug interactions. The average number of total hydrogen bonds was 13.3±2.7 and 1.9±1.4 for Sorafenib and Taselisib simulations, respectively. Moreover, the number of dye-drug hydrogen bonds was 10.3±2.6 and 0.4±0.6 for IR783-Sorafenib and IR783-Taselisib, respectively.

### Molecular Structures

Table 5 identifies molecular structures of drugs listed in Table 3.

TABLE 5
XL388

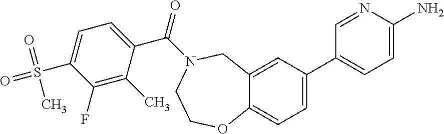 [SMILES: Cc1c(C(=O)N2CCOc3ccc(-c4ccc(N)nc4)cc3C2)ccc(S(C)(=O)=O)c1F]

RO5126766

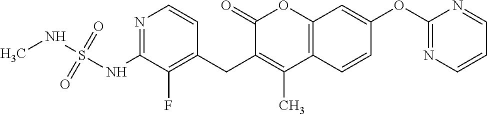 [SMILES: CNS(=O)(=O)Nc1nccc(Cc2c(C)c3ccc(Oc4ncccn4)cc3oc2=O)c1F]

Remikiren

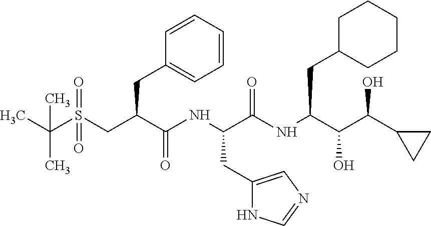 [SMILES: CC(C)(C)S(=O)(=O)C[C@@H](Cc1ccccc1)C(=O)N[C@@H](Cc1cnc[nH]1)C(=O)N[C@@H](CC1CCCCC1)[C@@H](O)[C@@H](O)C1CC1]

SU11274

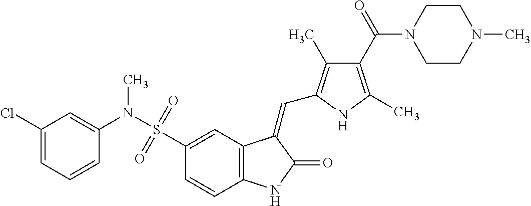 [SMILES: Cc1[nH]c(/C=C2\C(=O)Nc3ccc(S(=O)(=O)N(C)c4cccc(Cl)c4)cc32)c(C)c1C(=O)N1CCN(C)CC1]

Encorafenib

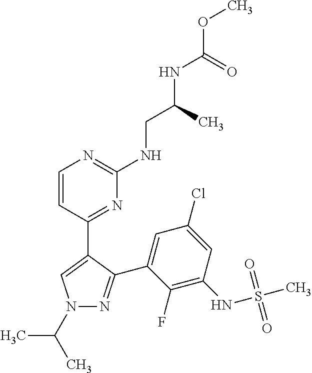 [SMILES: COC(=O)N[C@@H](C)CNc1nccc(-c2cn(C(C)C)nc2-c2cc(Cl)cc(NS(C)(=O)=O)c2F)n1]

C646

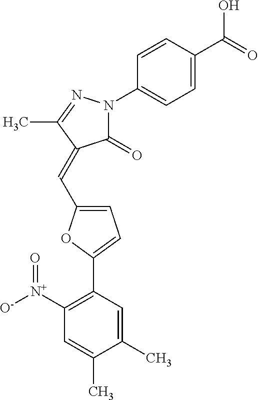 [SMILES: CC1=NN(c2ccc(C(=O)O)cc2)C(=O)/C1=C\c1ccc(-c2cc(C)c(C)cc2[N+](=O)[O-])o1]

PHA-665752

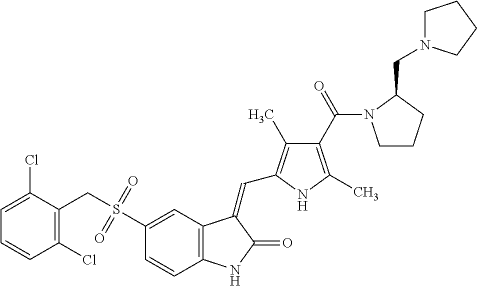 [SMILES: Cc1[nH]c(/C=C2\C(=O)Nc3ccc(S(=O)(=O)Cc4c(Cl)cccc4Cl)cc32)c(C)c1C(=O)N1CCC[C@@H]1CN1CCCC1]

Venetoclaxl

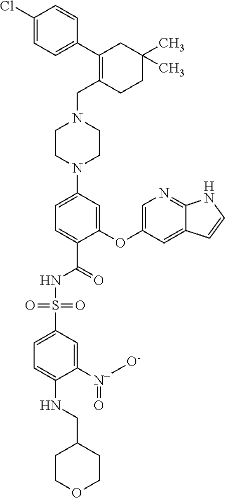 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

ABT737

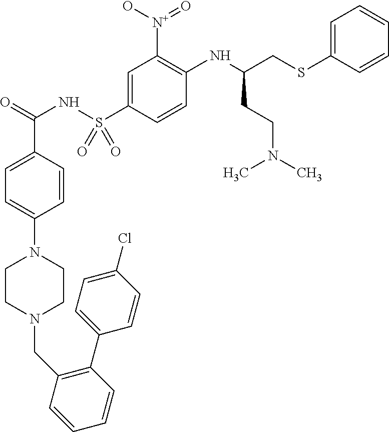

Tacrolimus

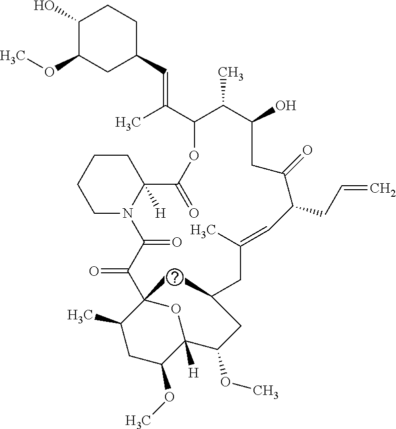 [SMILES: C=CC[C@@H]1/C=C(\C)C[C@H](C)C[C@H](OC)[C@H]2O[C@@](C)(C(=O)C(=O)N3CCCC[C@H]3C(=O)OC(/C(C)=C/[C@@H]3CC[C@@H](O)[C@H](OC)C3)[C@H](C)[C@@H](O)CC1=O)[C@H](C)C[C@@H]2OC]

Lansoprazole

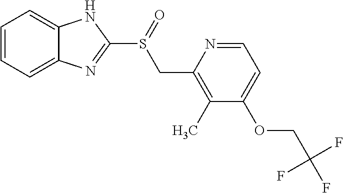 [SMILES: Cc1c(OCC(F)(F)F)ccnc1CS(=O)c1nc2ccccc2[nH]1]

AS-604850

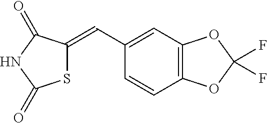 [SMILES: O=C1NC(=O)/C(=C/c2ccc3c(c2)OC(F)(F)O3)S1]

BMS-777607

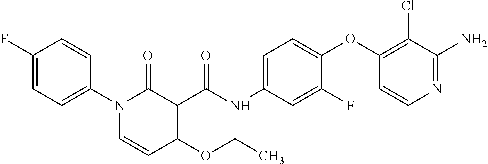 [SMILES: CCOC1C=CN(c2ccc(F)cc2)C(=O)C1C(=O)Nc1ccc(Oc2ccnc(N)c2Cl)c(F)c1]

Telithromycin

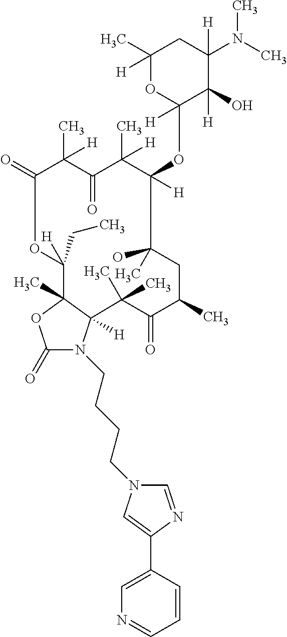

Foretinib

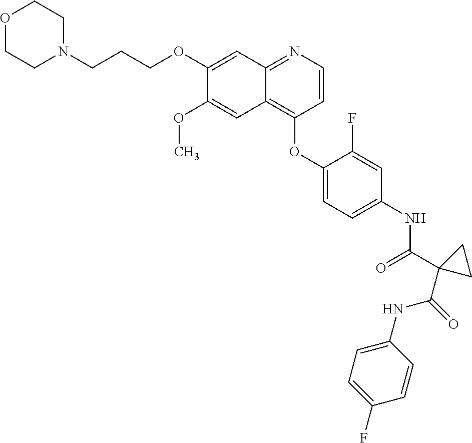 [SMILES: COc1cc2c(Oc3ccc(NC(=O)C4(C(=O)Nc5ccc(F)cc5)CC4)cc3F)ccnc2cc1OCCCN1CCOCC1]

Floxapen

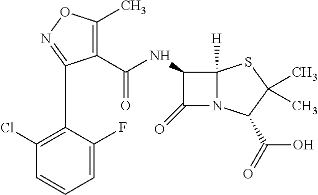 [SMILES: Cc1onc(-c2c(F)cccc2Cl)c1C(=O)N[C@@H]1C(=O)N2[C@@H]1SC(C)(C)[C@@H]2C(=O)O]

Carfilzomib

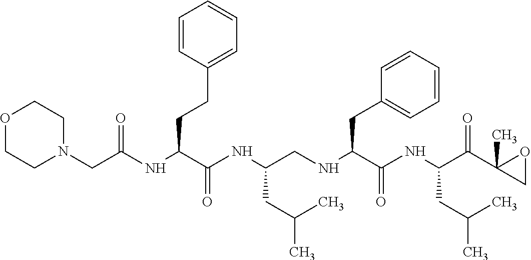 [SMILES: CC(C)C[C@@H](CN[C@@H](Cc1ccccc1)C(=O)N[C@@H](CC(C)C)C(=O)[C@@]1(C)CO1)NC(=O)[C@H](CCc1ccccc1)NC(=O)CN1CCOCC1]

Zotarolimus

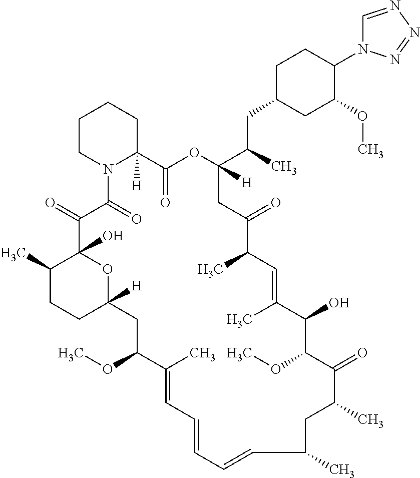 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CCC(n3cnnn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Everolimus

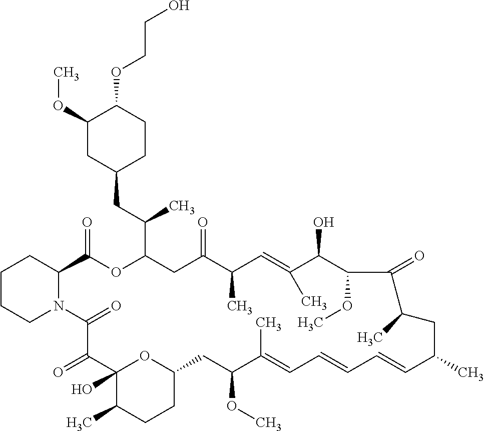 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

Rapamycin

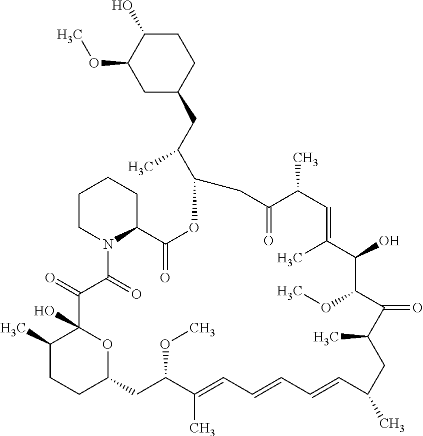 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

Telaprevir

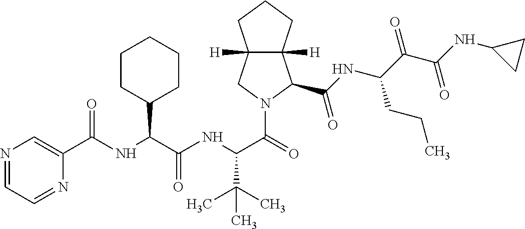 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]

Balifomycin

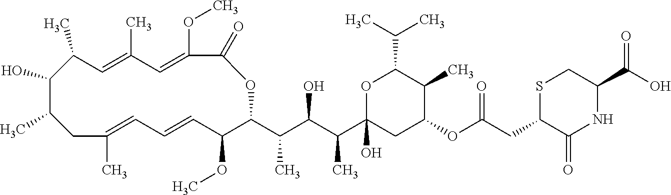 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](OC(=O)C[C@@H]3SC[C@@H](C(=O)O)NC3=O)[C@H](C)[C@@H](C(C)C)O2)OC1=O]

Temsirolimus

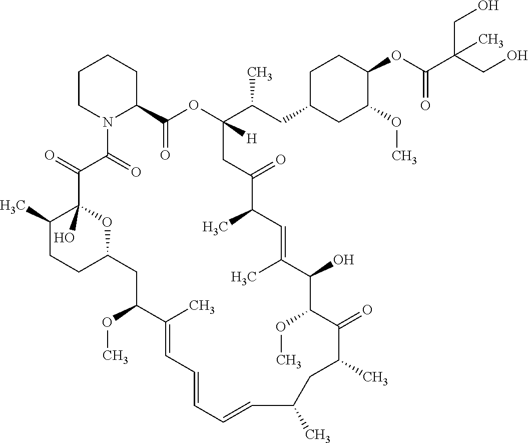 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)C(C)(CO)CO)[C@H](OC)C2)CC(=O)[C@H](C)/C=C(\C)[C@@H](O)[C@@H](OC)C(=O)[C@H](C)C[C@H](C)/C=C/C=C/C=C/1C]

LY411575

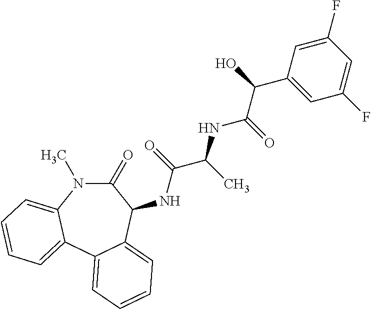 [SMILES: C[C@H](NC(=O)[C@@H](O)c1cc(F)cc(F)c1)C(=O)N[C@@H]1C(=O)N(C)c2ccccc2-c2ccccc21]

Trametinib

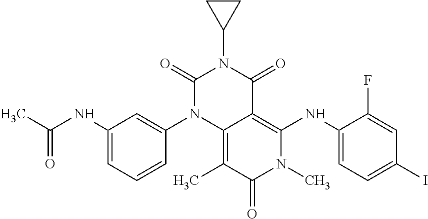 [SMILES: CC(=O)Nc1cccc(-n2c(=O)n(C3CC3)c(=O)c3c(Nc4ccc(I)cc4F)n(C)c(=O)c(C)c32)c1]

Lubiprostone

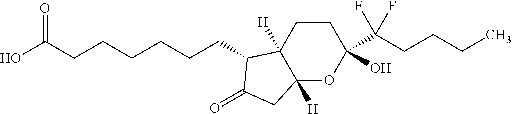 [SMILES: CCCCC(F)(F)[C@@]1(O)CC[C@H]2[C@@H](CC(=O)[C@@H]2CCCCCCC(=O)O)O1]

Cobimetinib

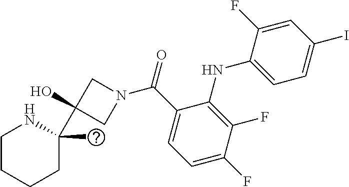

PLX-4720

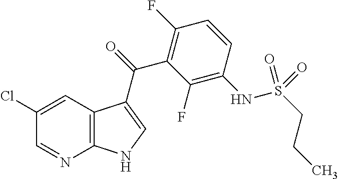 [SMILES: CCCS(=O)(=O)Nc1ccc(F)c(C(=O)c2c[nH]c3ncc(Cl)cc23)c1F]

Vemurafenib

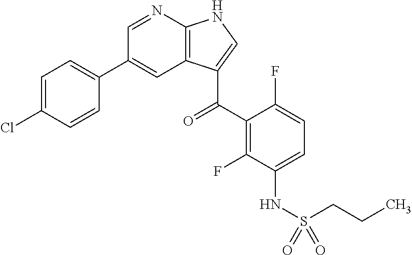 [SMILES: CCCS(=O)(=O)Nc1ccc(F)c(C(=O)c2c[nH]c3ncc(-c4ccc(Cl)cc4)cc23)c1F]

Verteporfin

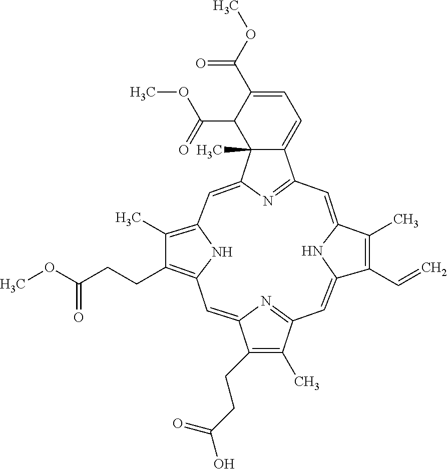 [SMILES: C=Cc1c(C)c2cc3nc(cc4[nH]c(cc5nc(cc1[nH]2)C(C)=C5CCC(=O)O)c(CCC(=O)OC)c4C)[C@@]1(C)C3=CC=C(C(=O)OC)C1C(=O)OC]

Viomycin

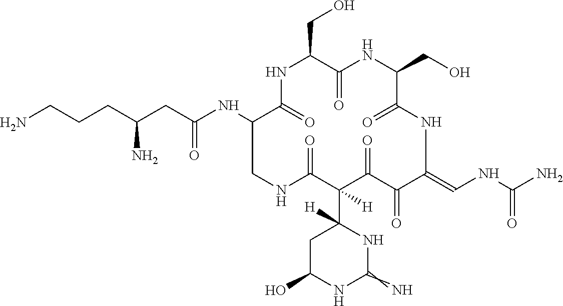 [SMILES: N=C1N[C@@H](O)C[C@H]([C@H]2C(=O)NCC(NC(=O)C[C@@H](N)CCCN)C(=O)N[C@@H](CO)C(=O)N[C@@H](CO)C(=O)N/C(=C\NC(N)=O)C(=O)C2=O)N1]

PND-1186

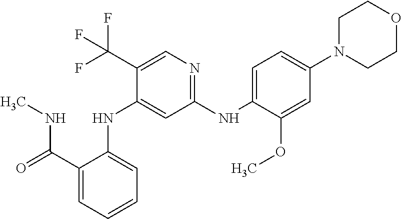 [SMILES: CNC(=O)c1ccccc1Nc1cc(Nc2ccc(N3CCOCC3)cc2OC)ncc1C(F)(F)F]

NVP-BHG712

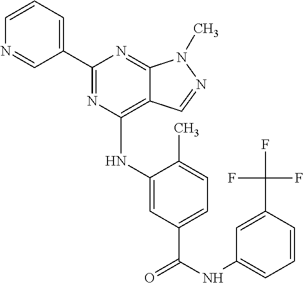 [SMILES: Cc1ccc(C(=O)Nc2cccc(C(F)(F)F)c2)cc1Nc1nc(-c2cccnc2)nc2c1cnn2C]

Nilotinib

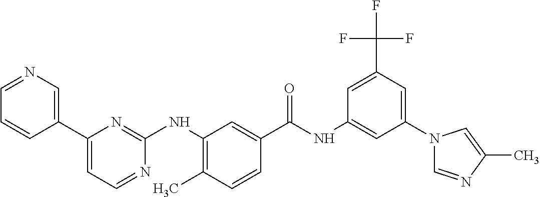 [SMILES: Cc1cn(-c2cc(NC(=O)c3ccc(C)c(Nc4nccc(-c5cccnc5)n4)c3)cc(C(F)(F)F)c2)cn1]

OSI-930

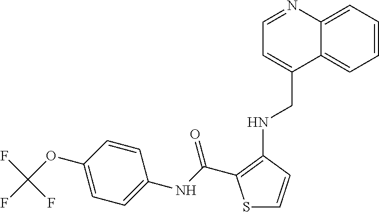 [SMILES: O=C(Nc1ccc(OC(F)(F)F)cc1)c1sccc1NCc1ccnc2ccccc12]

Troleandomycin

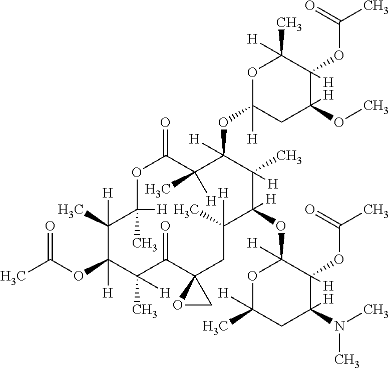 [SMILES: CO[C@H]1C[C@H](O[C@H]2[C@H](C)[C@@H](O[C@@H]3O[C@H](C)C[C@H](N(C)C)[C@H]3OC(C)=O)[C@@H](C)C[C@]3(CO3)C(=O)[C@H](C)[C@@H](OC(C)=O)[C@@H](C)[C@@H](C)OC(=O)[C@@H]2C)O[C@@H](C)[C@@H]1OC(C)=O]

Fluticasone

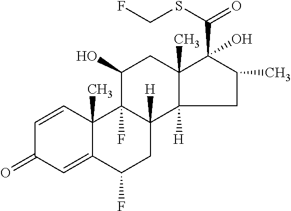

CH4987655

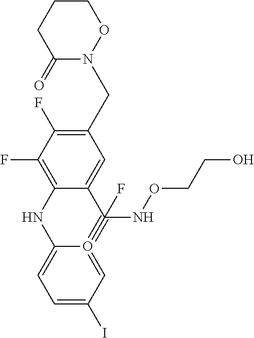

Paclitaxel

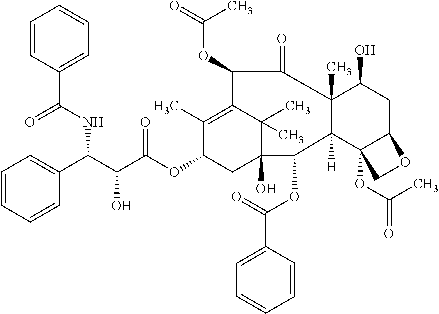 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Docetaxel

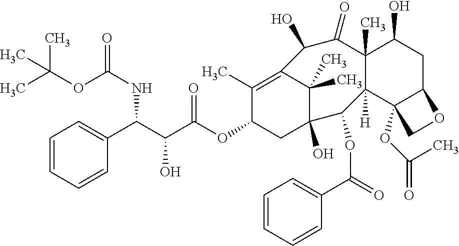

Pluripotin

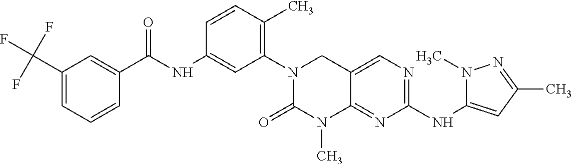 [SMILES: Cc1cc(Nc2ncc3c(n2)N(C)C(=O)N(c2cc(NC(=O)c4cccc(C(F)(F)F)c4)ccc2C)C3)n(C)n1]

CEP-32496

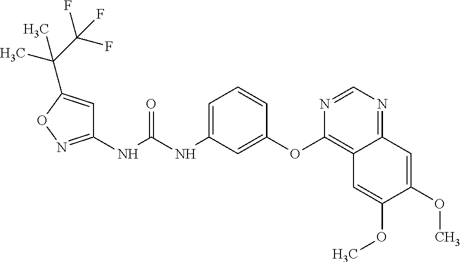 [SMILES: COc1cc2ncnc(Oc3cccc(NC(=O)Nc4cc(C(C)(C)C(F)(F)F)on4)c3)c2cc1OC]

Sorafenib

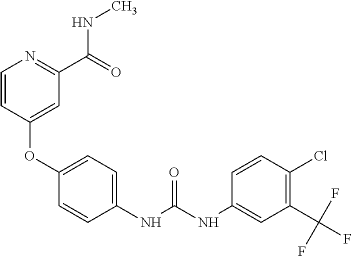 [SMILES: CNC(=O)c1cc(Oc2ccc(NC(=O)Nc3ccc(Cl)c(C(F)(F)F)c3)cc2)ccn1]

PF-03814735

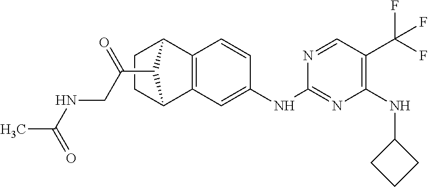 [SMILES: CC(=O)NCC(=O)C1[C@@H]2CC[C@H]1c1ccc(Nc3ncc(C(F)(F)F)c(NC4CCC4)n3)cc12]

Efavirenz

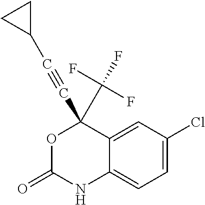

PF-04929113

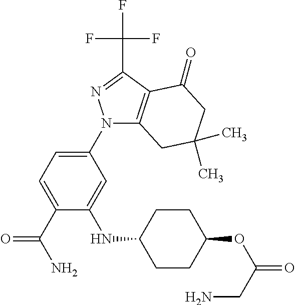 [SMILES: CC1(C)CC(=O)c2c(C(F)(F)F)nn(-c3ccc(C(N)=O)c(N[C@H]4CC[C@H](OC(=O)CN)CC4)c3)c2C1]

Valrubicin

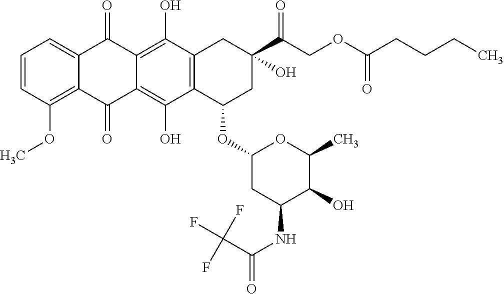 [SMILES: CCCCC(=O)OCC(=O)[C@]1(O)Cc2c(O)c3c(c(O)c2[C@@H](O[C@H]2C[C@H](NC(=O)C(F)(F)F)[C@H](O)[C@H](C)O2)C1)C(=O)c1c(OC)cccc1C3=O]

MK-0752

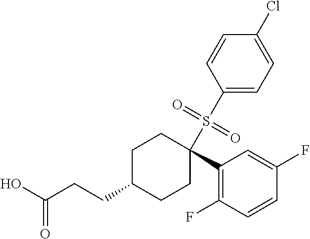 [SMILES: O=C(O)CC[C@H]1CC[C@@](c2cc(F)ccc2F)(S(=O)(=O)c2ccc(Cl)cc2)CC1]

GSK2636771

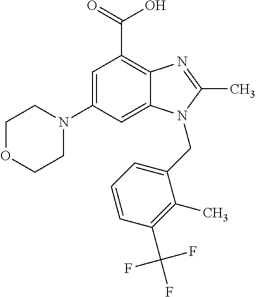 [SMILES: Cc1c(Cn2c(C)nc3c(C(=O)O)cc(N4CCOCC4)cc32)cccc1C(F)(F)F]

Saprisartan

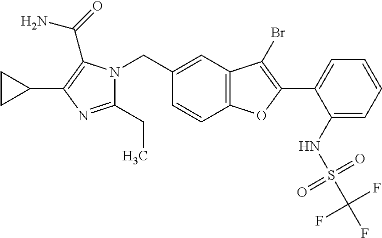 [SMILES: CCc1nc(C2CC2)c(C(N)=O)n1Cc1ccc2oc(-c3ccccc3NS(=O)(=O)C(F)(F)F)c(Br)c2c1]

Defactinib

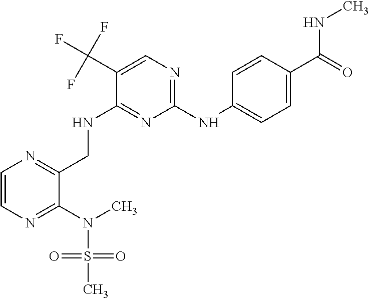 [SMILES: CNC(=O)c1ccc(Nc2ncc(C(F)(F)F)c(NCc3nccnc3N(C)S(C)(=O)=O)n2)cc1]

PF-00562271

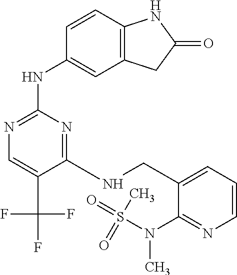 [SMILES: CN(c1ncccc1CNc1nc(Nc2ccc3c(c2)CC(=O)N3)ncc1C(F)(F)F)S(C)(=O)=O]

PF-431396

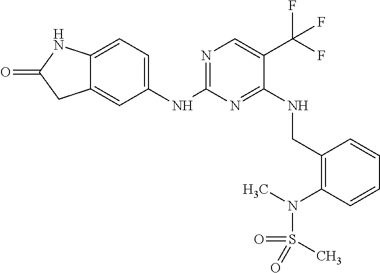 [SMILES: CN(c1ccccc1CNc1nc(Nc2ccc3c(c2)CC(=O)N3)ncc1C(F)(F)F)S(C)(=O)=O]

PF-573228

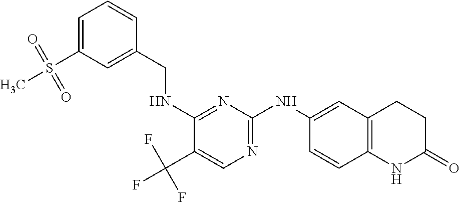 [SMILES: CS(=O)(=O)c1cccc(CNc2nc(Nc3ccc4c(c3)CCC(=O)N4)ncc2C(F)(F)F)c1]

Dabrafenib

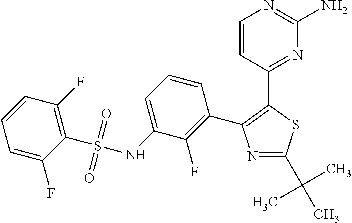 [SMILES: CC(C)(C)c1nc(-c2cccc(NS(=O)(=O)c3c(F)cccc3F)c2F)c(-c2ccnc(N)n2)s1]

Tipranavir

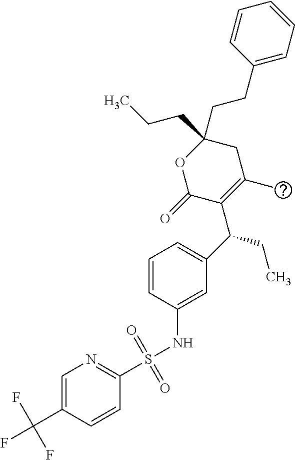 [SMILES: CCC[C@@]1(CCc2ccccc2)CC(C)=C([C@H](CC)c2cccc(NS(=O)(=O)c3ccc(C(F)(F)F)cn3)c2)C(=O)O1]

Navitoclax

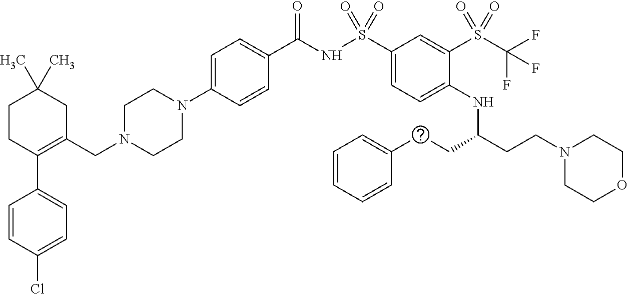 [SMILES: CC1(C)CCC(c2ccc(Cl)cc2)=C(CN2CCN(c3ccc(C(=O)NS(=O)(=O)c4ccc(N[C@@H](CCc5ccccc5)CCN5CCOCC5)c(S(=O)(=O)C(F)(F)F)c4)cc3)CC2)C1]

Celecoxib

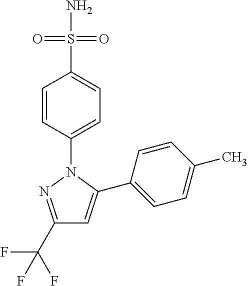 [SMILES: Cc1ccc(-c2cc(C(F)(F)F)nn2-c2ccc(S(N)(=O)=O)cc2)cc1]

Flutamide

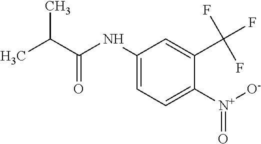 [SMILES: CC(C)C(=O)Nc1ccc([N+](=O)[O-])c(C(F)(F)F)c1]

Bicalutamide

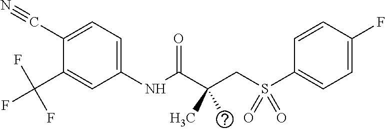

RO4929097

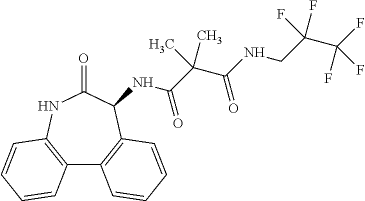 [SMILES: CC(C)(C(=O)NCC(F)(F)C(F)(F)F)C(=O)N[C@@H]1C(=O)Nc2ccccc2-c2ccccc21]

Avagacestat

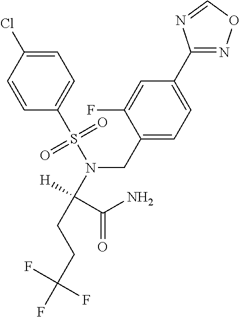 [SMILES: NC(=O)[C@@H](CCC(F)(F)F)N(Cc1ccc(-c2ncon2)cc1F)S(=O)(=O)c1ccc(Cl)cc1]

Mefloquine

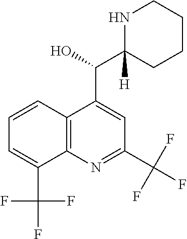 [SMILES: O[C@@H](c1cc(C(F)(F)F)nc2c(C(F)(F)F)cccc12)[C@H]1CCCCN1]

Enzalutamide

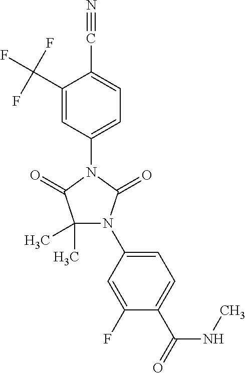 [SMILES: CNC(=O)c1ccc(N2C(=O)N(c3ccc(C#N)c(C(F)(F)F)c3)C(=O)C2(C)C)cc1F]

Regorafenib

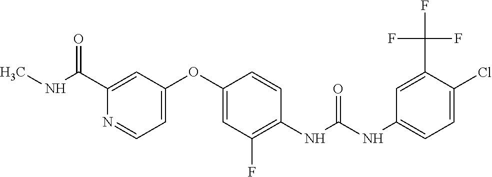 [SMILES: CNC(=O)c1cc(Oc2ccc(NC(=O)Nc3ccc(Cl)c(C(F)(F)F)c3)c(F)c2)ccn1]

Aprepitant

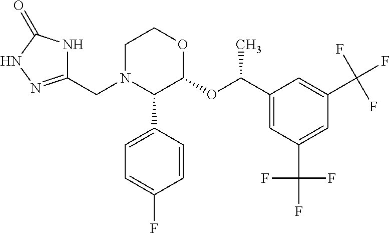 [SMILES: C[C@@H](O[C@H]1OCCN(Cc2n[nH]c(=O)[nH]2)[C@H]1c1ccc(F)cc1)c1cc(C(F)(F)F)cc(C(F)(F)F)c1]

Dutasteride

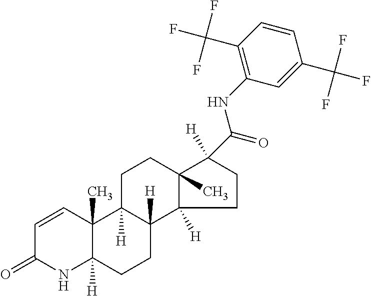

TAK-632

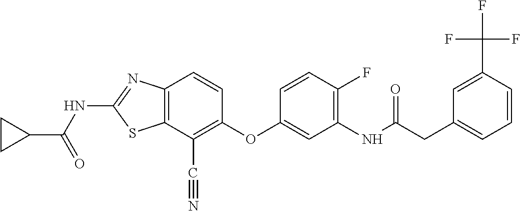 [SMILES: N#Cc1c(Oc2ccc(F)c(NC(=O)Cc3cccc(C(F)(F)F)c3)c2)ccc2nc(NC(=O)C3CC3)sc12]

antrafenine

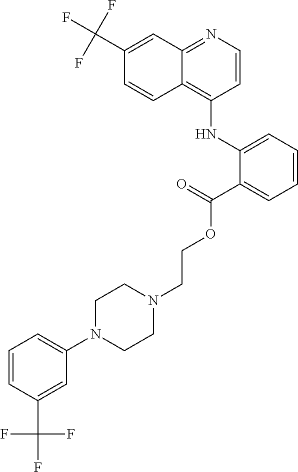 [SMILES: O=C(OCCN1CCN(c2cccc(C(F)(F)F)c2)CC1)c1ccccc1Nc1ccnc2cc(C(F)(F)F)ccc12]

Lomitapide

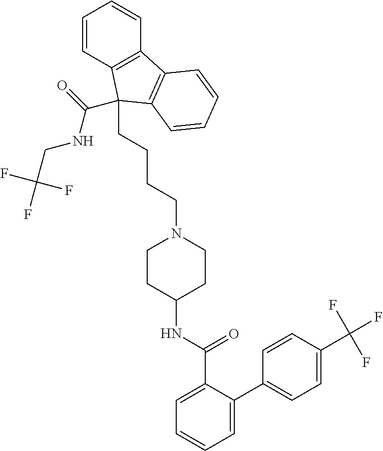 [SMILES: O=C(NC1CCN(CCCCC2(C(=O)NCC(F)(F)F)c3ccccc3-c3ccccc32)CC1)c1ccccc1-c1ccc(C(F)(F)F)cc1]

Fulvastrant

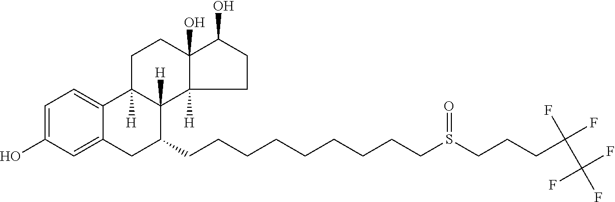

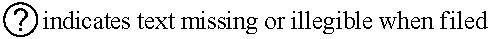

### Exemplary Dyes

Table 6 lists exemplary dyes that can be used in dye-stabilized nanoparticles as described herein.

TABLE 6

| Name | IUPAC | Chemical structure |
|---|---|---|
| IR783 | 2-[2-[2-Chloro-3-[2- [1,3-dihydro-3,3- dimethyl-1-(4- sulfobutyl)-2H-indol- 2-ylidene]- ethylidene]-1- cyclohexen-1-yl]- ethenyl]-3,3- dimethyl-1-(4- sulfobutyl)-3H- indolium hydroxide, inner salt sodium salt | 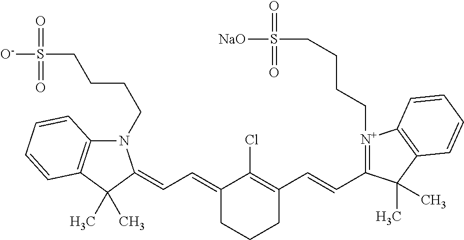 |
| IR806 | 2-[2-[2-chloro-3-[2- [1,3-dihydro-3,3- dimethyl-1-(4- sulfobutyl)-2H-indol- 2-ylidene]- ethylidene]-1- cyclopenten-1-yl]- ethenyl]-3,3- dimethyl-1-(4- sulfobutyl)-3H- indolium hydroxide, inner salt sodium salt | 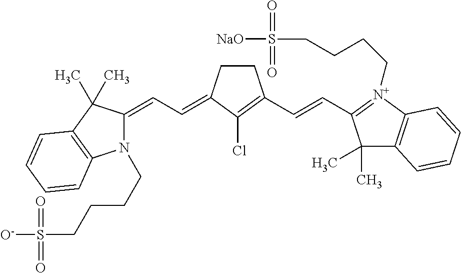 |
| IR820 | 2-[2-[2-Chloro-3- [[1,3-dihydro-1,1- dimethyl-3-(4- sulfobutyl)-2H- benzo[e]indol-2- yidene]-ethylidene]- 1-cyclohexen-1-yl]- ethenyl]-1,1- dimethyl-3-(4- sulfobutyl)-1H- benzo[e]indolium hydroxide inner salt, sodium salt, New Indocyanine Green | 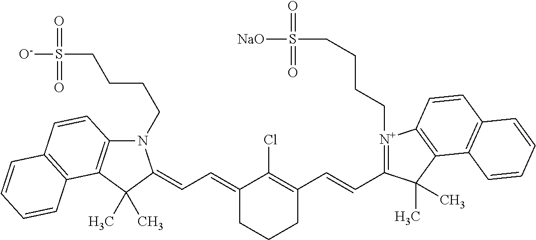 |
| IR775 | 2-[2-[2-Chloro-3-[2- (1,3-dihydro-1,3,3- trimethyl-2H-indol-2- ylidene)-ethylidene]- 1-cyclohexen-1-yl]- ethenyl]-1,3,3- trimethyl-3H- indolium chloride | 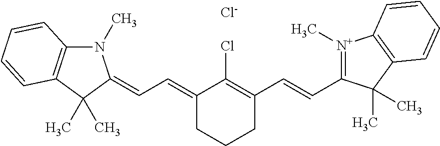 [SMILES: CN1/C(=C\C=C2/CCCC(/C=C/C3=[N+](C)c4ccccc4C3(C)C)=C2Cl)C(C)(C)c2ccccc21 ; [Cl-]] |
| IR780 iodide | 2-[2-[2-Chloro-3- [(1,3-dihydro-3,3- dimethyl-1-propyl- 2H-indol-2- ylidene)ethylidene]- 1-cyclohexen-1- yl]ethenyl]-3,3- dimethyl-1- propylindolium iodide | 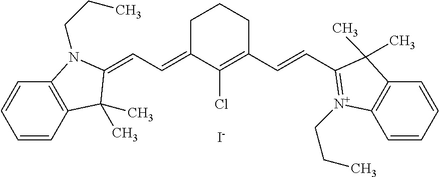 [SMILES: CCCN1/C(=C/C=C2\CCCC(/C=C/C3=[N+](CCC)c4ccccc4C3(C)C)=C2Cl)C(C)(C)c2ccccc21 ; [I-]] |
| IR780 percholate | 1,1′,3,3,3′,3′-4,4′,5,5′- di-benzo-2,2′- indotricarbocyanine perchlorate, HDITC perchlorate | 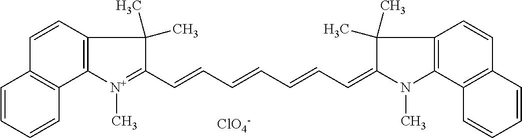 |
| IR125 | 2-[7-[1,3-dihydro- 1,1-dimethyl-3-(4- sulfobutyl)-2H- benz[e]indol-2- ylidene]1,3,5- heptatrienyl]-1,1- dimethyl-3-(4- sulfobutyl)-1H- benz[e]indolium hydroxide, inner salt, sodium salt | 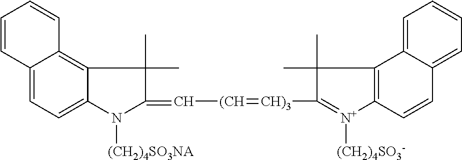 |

### Illustrative Network Environment and Computing Device

FIG. 34 shows an illustrative network environment 3400 for use in the methods and systems for analysis of spectrometry data corresponding to particles of a sample, as described herein. In brief overview, referring now to FIG. 34, a block diagram of an exemplary cloud computing environment 3400 is shown and described. The cloud computing environment 3400 may include one or more resource providers 3402a, 3402b, 3402c (collectively, 3402). Each resource provider 3402 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 3402 may be connected to any other resource provider 3402 in the cloud computing environment 3400. In some implementations, the resource providers 3402 may be connected over a computer network 3408. Each resource provider 3402 may be connected to one or more computing device 3404a, 3404b, 3404c (collectively, 3404), over the computer network 3408.

The cloud computing environment 3400 may include a resource manager 3406. The resource manager 3406 may be connected to the resource providers 3402 and the computing devices 3404 over the computer network 3408. In some implementations, the resource manager 3406 may facilitate the provision of computing resources by one or more resource providers 3402 to one or more computing devices 3404. The resource manager 3406 may receive a request for a computing resource from a particular computing device 3404. The resource manager 3406 may identify one or more resource providers 3402 capable of providing the computing resource requested by the computing device 3404. The resource manager 3406 may select a resource provider 3402 to provide the computing resource. The resource manager 3406 may facilitate a connection between the resource provider 3402 and a particular computing device 3404. In some implementations, the resource manager 3406 may establish a connection between a particular resource provider 3402 and a particular computing device 3404. In some implementations, the resource manager 3406 may redirect a particular computing device 3404 to a particular resource provider 3402 with the requested computing resource.

FIG. 35 shows an example of a computing device 3500 and a mobile computing device 3550 that can be used in the methods and systems described in this disclosure. The computing device 3500 is intended to represent various forms of digital computers, such as laptops, desktops, workstations, personal digital assistants, servers, blade servers, mainframes, and other appropriate computers. The mobile computing device 3550 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 3500 includes a processor 3502, a memory 3504, a storage device 3506, a high-speed interface 3508 connecting to the memory 3504 and multiple high-speed expansion ports 3510, and a low-speed interface 3512 connecting to a low-speed expansion port 3514 and the storage device 3506. Each of the processor 3502, the memory 3504, the storage device 3506, the high-speed interface 3508, the high-speed expansion ports 3510, and the low-speed interface 3512, are interconnected using various busses, and may be mounted on a common motherboard or in other manners as appropriate. The processor 3502 can process instructions for execution within the computing device 3500, including instructions stored in the memory 3504 or on the storage device 3506 to display graphical information for a GUI on an external input/output device, such as a display 3516 coupled to the high-speed interface 3508. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 3504 stores information within the computing device 3500. In some implementations, the memory 3504 is a volatile memory unit or units. In some implementations, the memory 3504 is a non-volatile memory unit or units. The memory 3504 may also be another form of computer-readable medium, such as a magnetic or optical disk.

The storage device 3506 is capable of providing mass storage for the computing device 3500. In some implementations, the storage device 3506 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 3502), perform one or more methods, such as those described above. The instructions can also be stored by one or more storage devices such as computer- or machine-readable mediums (for example, the memory 3504, the storage device 3506, or memory on the processor 3502).

The high-speed interface 3508 manages bandwidth-intensive operations for the computing device 3500, while the low-speed interface 3512 manages lower bandwidth-intensive operations. Such allocation of functions is an example only. In some implementations, the high-speed interface 3508 is coupled to the memory 3504, the display 3516 (e.g., through a graphics processor or accelerator), and to the high-speed expansion ports 3510, which may accept various expansion cards (not shown). In the implementation, the low-speed interface 3512 is coupled to the storage device 3506 and the low-speed expansion port 3514. The low-speed expansion port 3514, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 3500 may be implemented in a number of different forms, as shown in the figure. For example, it may be implemented as a standard server 3520, or multiple times in a group of such servers. In addition, it may be implemented in a personal computer such as a laptop computer 3522. It may also be implemented as part of a rack server system 3524. Alternatively, components from the computing device 3500 may be combined with other components in a mobile device (not shown), such as a mobile computing device 3550. Each of such devices may contain one or more of the computing device 3500 and the mobile computing device 3550, and an entire system may be made up of multiple computing devices communicating with each other.

The mobile computing device 3550 includes a processor 3552, a memory 3564, an input/output device such as a display 3554, a communication interface 3566, and a transceiver 3568, among other components. The mobile computing device 3550 may also be provided with a storage device, such as a micro-drive or other device, to provide additional storage. Each of the processor 3552, the memory 3564, the display 3554, the communication interface 3566, and the transceiver 3568, are interconnected using various buses, and several of the components may be mounted on a common motherboard or in other manners as appropriate.

The processor 3552 can execute instructions within the mobile computing device 3550, including instructions stored in the memory 3564. The processor 3552 may be implemented as a chipset of chips that include separate and multiple analog and digital processors. The processor 3552 may provide, for example, for coordination of the other components of the mobile computing device 3550, such as control of user interfaces, applications run by the mobile computing device 3550, and wireless communication by the mobile computing device 3550.

The processor 3552 may communicate with a user through a control interface 3558 and a display interface 3556 coupled to the display 3554. The display 3554 may be, for example, a TFT (Thin-Film-Transistor Liquid Crystal Display) display or an OLED (Organic Light Emitting Diode) display, or other appropriate display technology. The display interface 3556 may comprise appropriate circuitry for driving the display 3554 to present graphical and other information to a user. The control interface 3558 may receive commands from a user and convert them for submission to the processor 3552. In addition, an external interface 3562 may provide communication with the processor 3552, so as to enable near area communication of the mobile computing device 3550 with other devices. The external interface 3562 may provide, for example, for wired communication in some implementations, or for wireless communication in other implementations, and multiple interfaces may also be used.

The memory 3564 stores information within the mobile computing device 3550. The memory 3564 can be implemented as one or more of a computer-readable medium or media, a volatile memory unit or units, or a non-volatile memory unit or units. An expansion memory 3574 may also be provided and connected to the mobile computing device 3550 through an expansion interface 3572, which may include, for example, a SIMM (Single In Line Memory Module) card interface. The expansion memory 3574 may provide extra storage space for the mobile computing device 3550, or may also store applications or other information for the mobile computing device 3550. Specifically, the expansion memory 3574 may include instructions to carry out or supplement the processes described above, and may include secure information also. Thus, for example, the expansion memory 3574 may be provided as a security module for the mobile computing device 3550, and may be programmed with instructions that permit secure use of the mobile computing device 3550. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 3552), perform one or more methods, such as those described above. The instructions can also be stored by one or more storage devices, such as one or more computer- or machine-readable mediums (for example, the memory 3564, the expansion memory 3574, or memory on the processor 3552). In some implementations, the instructions can be received in a propagated signal, for example, over the transceiver 3568 or the external interface 3562.

The mobile computing device 3550 may communicate wirelessly through the communication interface 3566, which may include digital signal processing circuitry where necessary. The communication interface 3566 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 3568 using a radio-frequency. In addition, short-range communication may occur, such as using a Bluetooth®, Wi-Fi™, or other such transceiver (not shown). In addition, a GPS (Global Positioning System) receiver module 3570 may provide additional navigation- and location-related wireless data to the mobile computing device 3550, which may be used as appropriate by applications running on the mobile computing device 3550.

The mobile computing device 3550 may also communicate audibly using an audio codec 3560, which may receive spoken information from a user and convert it to usable digital information. The audio codec 3560 may likewise generate audible sound for a user, such as through a speaker, e.g., in a handset of the mobile computing device 3550. Such sound may include sound from voice telephone calls, may include recorded sound (e.g., voice messages, music files, etc.) and may also include sound generated by applications operating on the mobile computing device 3550.

The mobile computing device 3550 may be implemented in a number of different forms, as shown in the figure. For example, it may be implemented as a cellular telephone 3580. It may also be implemented as part of a smart-phone 3582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

1. A dye-stabilized nanoparticle composition comprising
at least about 80 wt. % of one or more hydrophobic drugs; and
one or more sulfate-containing indocyanine dyes;
wherein the composition is in the form of nanoparticles having an intensity-weighted average diameter as determined by dynamic light scattering within a range from 30 nm to 150 nm.

2. The dye-stabilized nanoparticle composition of claim 1, wherein the composition comprises at least 80 wt. % of the one or more hydrophobic drugs.

3. The dye-stabilized nanoparticle composition of claim 1, wherein the composition comprises about 5 wt. % to about 20 wt. % of the one or more sulfate-containing indocyanine dyes.

4. The dye-stabilized nanoparticle composition of claim 1, wherein the an intensity-weighted average diameter of the nanoparticles is within a range from 40 nm to 100 nm.

5-6. (canceled)

7. The dye-stabilized nanoparticle composition of claim 1, wherein the one or more hydrophobic drugs comprise a fluorine covalently bound to each of the one or more hydrophobic drugs.

8. The dye-stabilized nanoparticle composition of claim 1, wherein the one or more hydrophobic drugs comprise one or more members selected from the group consisting of sorafenib, paclitaxel, docetaxel, MEK162, etoposide, lapatinib, nilotinib, crizotinib, fulvestrant, vemurafenib, bexorotene, camptothecin, Mek Azd, talazoparib, GSK214, luminespib, forskolin, ABT737, tacrolimus, BMS-777607, tanespimycin, everolimus, trametinib, navitoclax, celecoxib, avagacestat, dutasteride, enzalutamide, regorafenib, R04929097, valrubicin, and combinations of any two or more thereof.

9. (canceled)

10. The dye-stabilized nanoparticle composition of claim 1, wherein the one or more sulfate-containing indocyanine dyes comprise one or more members selected from the group consisting of IR783, IR806, IR820, IR125, and combinations of any two or more thereof.

11. The dye-stabilized nanoparticle composition of claim 1, wherein the one or more sulfate-containing indocyanine dyes comprise IR783.

12-14. (canceled)

15. The dye-stabilized nanoparticle composition of claim 1, wherein the nanoparticles exhibit a zeta potential from −20 mV to −100 mV.

16. The dye-stabilized nanoparticle composition of claim 1, wherein the one or more hydrophobic drugs are not covalently bonded to the one or more sulfate-containing indocyanine dyes.

17-18. (canceled)

19. A method of treating a disease or condition, the method comprising administering the dye-stabilized nanoparticle composition of claim 1 to a subject suffering from or susceptible to the disease or condition; wherein the disease or condition is selected from the group consisting of cancer, inflammatory disease, rheumatoid arthritis, inflammatory bowel disease, lupus, and age-related macular degeneration.

20. (canceled)

21. The method of claim 19, wherein the administered dye-stabilized nanoparticle composition obviate skin rashes.

22. The method of claim 19, wherein the method further comprises irradiating the dye-stabilized nanoparticle composition subsequent to the administering.

23. A method of making the dye-stabilized nanoparticle composition of claim 1, the method comprising
introducing a first solution into a second solution in a drop-wise manner while agitating the second solution;
wherein the first solution comprises the one or more hydrophobic drugs in a solvent, and the second solution is a buffered aqueous solution comprising the one or more sulfate-containing indocyanine dyes.

24. The method of claim 23, wherein the solvent comprises DMSO, ethanol, or a combination thereof.

25. The method of claim 23, wherein the second solution has a total concentration of about 1 mg/ml to about 3 mg/ml of the one or more sulfate-containing indocyanine dyes.

26. The method of claim 23, wherein the method further comprises performing centrifugation and/or sonication to collect the formed nanoparticles.

27. A method for predicting self-assembly of a dye-stabilized nanoparticle composition, the method comprising
providing a molecular structure of a drug;
generating, by a computer program a set of one or more molecular descriptors for the drug, wherein the set of molecular descriptors comprises one or more of (i), (ii), (iii), and (iv) as follows:
(i) a first molecular descriptor identifying a likelihood the drug will self-assemble with a dye to generate a dye-stabilized nanoparticle composition comprising the drug and the dye;
(ii) a second molecular descriptor identifying a maximal quantity of drug that can be loaded into a/the dye-stabilized nanoparticle composition comprising the drug and the dye;
(iii) a third molecular descriptor identifying hydrophobicity of the drug; and
(iv) a fourth molecular descriptor identifying a diameter of a/the dye-stabilized nanoparticle composition comprising the drug and the dye.

28-37. (canceled)

38. The dye-stabilized nanoparticle composition of claim 1, wherein the composition further comprises one or more members selected from the group consisting of IR775, IR780, or a combination thereof.

39. The dye-stabilized nanoparticle composition of claim 1, wherein the composition exhibits a polydispersity index of about 0.05 to about 0.3.
